(19) United States
(12) Patent Application Publication
(10) Pub. No.: US 2009/0221653 A1
(43) Pub. Date: Sep. 3, 2009

(54) 7-(2-amino-1-hydroxy-ethyl)-4-hydroxybenzothiazol-2(3H)-one-derivatives as beta2 adrenoreceptor agonists

(75) Inventors: Andrew Bailey, Leicestershire (GB); Roger Bonnert, Leicestershire (GB); Stephen Connolly, Leicestershire (GB); Anthony Ingall, Leicestershire (GB); Garry Pairaudeau, Leicestershire (GB); Michael Stocks, Leicestershire (GB)

Correspondence Address: FISH & RICHARDSON P.C., P.O BOX 1022, MINNEAPOLIS, MN, 55440-1022, US

(73) Assignee: AstraZeneca AB, Sodertalje (SE)

(21) Appl. No.: 12/065,160

(22) PCT Filed: Aug. 28, 2006
(86) PCT No.: PCT/SE06/00981

§ 371 Date: Jul. 29, 2008

(30) Foreign Application Priority Data
Aug. 29, 2005 (SE) 0501905-4
Jun. 15, 2006 (SE) 0601331-2

(51) Int. Cl. A61K 31/428; C07D 277/68; A61P 29/00

(52) U.S. Cl. 514/367; 548/169

(57) ABSTRACT

The present invention provides compounds of formula (I) wherein the variables are as defined in the specification, processes for their preparation, pharmaceutical compositions containing them and their use in therapy.

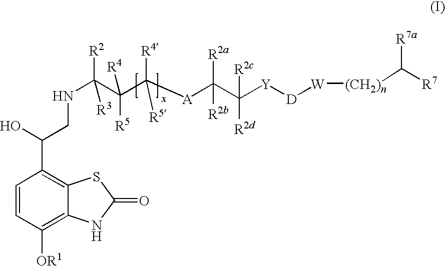

The present invention relates to benzothiazolone derivatives, processes for their preparation, pharmaceutical compositions containing them and their use in therapy.

Adreneoceptors are a group of G-protein coupled receptors divided into two major sub-families, α and β. These sub-families are further divided into sub-types of which the β sub-family has at least 3 members: β1, β2 and β3. β2 adrenoceptors (henceforth referred to as β2 receptors) are mainly expressed on smooth muscle cells.

Agonism of the β2 receptor on airway smooth muscle produces relaxation and therefore bronchodilatation. Through this mechanism, β2 agonists act as functional antagonists to all bronchoconstrictor substances such as the naturally-occurring histamine and acetylcholine as well as the experimental substances methacholine and carbachol. β2 agonists are widely used to treat airways diseases including asthma and chronic obstructive pulmonary disease (COPD), and this has been extensively reviewed in the literature and incorporated into national guidelines for the treatment of these diseases (British Guideline on the Management of Asthma, NICE guideline No. 12 on the Management of COPD).

β2 agonists are classed either as short-acting or long-acting. Short-acting β2 agonists (SABAs) such as salbutamol have a duration of action of 2-4 hours. They are suitable for rescue medication during a period of acute bronchoconstriction but are not suitable for continuous medication because the beneficial effect of these drugs wears off during the night. Long-acting β2 agonists (LABAs) currently have a duration of action of about 12 hours and are administered twice daily to provide continuous bronchodilatation. They are particularly effective when administered in combination with inhaled corticosteroids. This benefit is not seen when inhaled corticosteroids are combined with SABAs (Kips and Pauwels, Am. J. Respir. Crit. Care Med., 2001, 164, 923-932). LABAs are recommended as add-on therapy to patients already receiving inhaled corticosteroids for asthma to reduce nocturnal awakening and reduce the incidence of exacerbations of the disease.

Corticosteroids and LABAs are conveniently co-administered in a single inhaler to improve patient compliance.

There are shortcomings to existing LABAs and there is a need for a new drug in this class. Salmeterol, a commonly used LABA, has a narrow safety margin and side effects related to systemic agonism of β2 receptors (such as tremor, hypokalemia, tachycardia and hypertension) are common. Salmeterol also has a long onset of action which precludes its use as both a rescue and a maintenance therapy. All current LABAs are administered twice daily and there is a medical need for once daily treatments to improve treatment and patient compliance. Such once daily compounds, co-administered with corticosteroids, will become the mainstay of asthma treatment (Barnes, Nature Reviews, 2004, 3, 831-844). The advantages of once-daily bronchodilator treatment in COPD has been demonstrated with tiotropium, a non-selective muscarinic antagonist (Koumis and Samuel, Clin. Ther. 2005, 27(4), 377-92). There is, however, a need for a once-daily LABA for the treatment of COPD to avoid the side effects of anti-muscarinics such as tiotropium.

Benzothiazolone derivatives having dual β2 receptor and dopamine (D2) receptor agonist properties are known from WO 92/08708, WO 93/23385, WO 93/24473, WO 97/10227 and WO 97/23470. β2 Receptor agonists are disclosed in WO 2004/071388.

In accordance with the present invention, there is therefore provided a compound of formula (I):

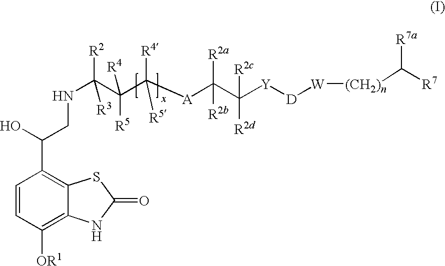

wherein
R¹ represents hydrogen;
each of R², R³, R⁴, R⁵, R^{4′} and R^{5′} independently represents hydrogen or C₁-C₆ alkyl;
x is 0 or 1;
A represents oxygen, sulphur, S(O) or S(O)₂;
D represents oxygen, sulphur or NR⁶;
W is a bond or CR^{6a}R^{6b};
n is an integer from 0 to 2;
R⁶ represents hydrogen, C₁-C₆ alkyl, C₁-C₆ alkoxycarbonyl or arylC₁-C₆alkyl;
Y is a bond, CR^{2e}R^{2f} or CR^{2g}R^{2h}CR^{2k}R^{2m};
R^{2a}, R^{2b}, R^{2c}, R^{2d}, R^{2e}, R^{2f}, R^{2g}, R^{2h}, R^{2k}, R^{2m}, R^{6a} and R^{6b} are, independently, hydrogen or C₁-C₆ alkyl;
R^{7a} is hydrogen, C₁-C₆ alkyl or NHR^{7b};
R^{7b} is hydrogen, C₁-C₆ alkyl, C₁-C₆ alkoxycarbonyl or arylC₁-C₆alkyl;
R⁷ represents a 5- to 14-membered aromatic or heteroaromatic ring system optionally substituted by halogen, trifluoromethyl, hydroxyl, carboxyl, C₁-C₆ alkyl (optionally substituted by —NR¹⁰R¹¹), C₁₋C₆ alkoxy (optionally substituted by —NR¹²R¹³), C₁-C₆ alkoxycarbonyl, —NR¹⁴R¹⁵, C₁-C₆ alkylcarbonylamino, C₁-C₆ alkylsulphonylamino, phenylsulphonylamino, —C(O)NHR¹⁶, —SO₂NHR¹⁷, C₀-C₆ alkyl-R¹⁸, or a phenyl or 5- to 6-membered heteroaromatic ring (each of which is optionally substituted by halogen, trifluoromethyl, hydroxyl, C₁-C₆ alkyl, C₁-C₆ alkoxy or NR²¹R²²);
R¹⁰, R¹¹, R¹², R¹³, R¹⁴ and R¹⁵ each independently represent hydrogen or C₁-C₆ alkyl;
R¹⁶ represents hydrogen, C₁-C₆ alkyl, phenyl-C₀-C₆ alkyl or C₂-C₆ alkylene-NR¹⁹R²⁰;
either R¹⁹ and R²⁰ each independently represent hydrogen or C₁-C₆ alkyl, or R¹⁹ and R²⁰ together with the nitrogen atom to which they are attached form a 4- to 6-membered saturated heterocyclic ring optionally comprising a further ring heteroatom selected from nitrogen and oxygen;
R¹⁷ represents hydrogen, C₁-C₆ alkyl, phenyl-C₀-C₆ alkyl or C₂-C₆ alkylene-NR²³R²⁴;
either R²³ and R²⁴ each independently represent hydrogen or C₁-C₆ alkyl, or R²³ and R²⁴ together with the nitrogen atom to which they are attached form a 4- to 6-membered saturated heterocyclic ring optionally comprising a further ring heteroatom selected from nitrogen and oxygen;
R¹⁸ represents a saturated, 5- or 6-membered nitrogen-containing ring; and
R²¹ and R²² each independently represent hydrogen or C₁-C₆ alkyl;
   or a pharmaceutically acceptable salt thereof.

Thus the invention provides a compound of formula (I) wherein R⁷ is a 5- to 14-membered aromatic or heteroaromatic ring system optionally substituted by one or more substituents independently selected from halogen, trifluoromethyl, hydroxyl, carboxyl, C₁-C₆ alkyl (optionally substituted by —NR¹⁰R¹¹), C₁-C₆ alkoxy (optionally substituted by —NR¹²R¹³), C₁-C₆ alkoxycarbonyl, —NR¹⁴R¹⁵, C₁-C₆ alkylcarbonylamino, C₁-C₆ alkylsulphonylamino, phenylsulphonylamino, —C(O)NHR⁶, —SO₂NHR¹⁷, C₀-C₆ alkyl-R¹⁸, and a phenyl or 5- to 6-membered heteroaromatic ring (each of which may be optionally substituted by one or more substituents independently selected from halogen, trifluoromethyl, hydroxyl, C₁-C₆ alkyl, C₁-C₆ alkoxy and —NR²¹R²²).

For the avoidance of doubt, when a group is described as ‘optionally substituted by’ or ‘may be optionally substituted by’, and whether such phrase is followed by a numerical qualification or not, said group can be unsubstituted or it can be substituted. When there is more than one substituent in the list of recited substituents and said group is substituted, said group can be substituted by the same or different substituents.

In the context of the present specification, unless otherwise stated, an alkyl substituent group or an alkyl moiety in a substituent group may be linear or branched. Examples of C₁-C₆ alkyl groups/moieties include methyl, ethyl, n-propyl, isopropyl, n-butyl, isobutyl, tert-butyl, n-pentyl and n-hexyl. Similarly, an alkylene group or an alkylene moiety in a substituent group may be linear or branched. Examples of C₁-C₆ alkylene groups/moieties include methylene, ethylene, n-propylene, n-butylene, n-pentylene, n-hexylene, 1-methylethylene, 2-methylethylene, 1,2-dimethylethylene, 1-ethylethylene, 2-ethylethylene, 1-, 2- or 3-methylpropylene and 1-, 2- or 3-ethylpropylene. An aryl group or an aryl moiety in a substituent group refers to a mono or fused bicyclic aromatic ring having from 6 to 10 carbon atoms. Monocyclic rings preferably have 6 members and bicyclic rings preferably have 8, 9 or 10 membered ring structures. Exemplary aryl groups/moieties include phenyl and naphthyl.

When R¹⁹ and R²⁰ (or R²³ and R²⁴) together represent a 4- to 6-membered saturated heterocyclic ring, it should be understood that the ring will contain no more than two ring heteroatoms: the nitrogen ring atom to which R¹⁹ and R²⁰ (or R²³ and R²⁴) are attached and optionally a nitrogen or oxygen ring atom.

The compounds of the invention are selective β2 receptor agonists and possess properties that make them more suitable for once-a-day administration. Compounds have been optimised to have appropriate duration in an in vitro guinea pig trachea model, or mammalian model such as a histamine-challenged guinea pig. The compounds also have advantageous pharmacokinetic half lives in mammalian systems. In particular, the compounds of the invention are at least 10-fold more potent at the β2 receptor compared to the α1, β1, or dopamine (D2) receptors. The compounds are also considered to have a fast onset of action that is the time interval between administration of a compound of the invention to a patient and the compound providing symptomatic relief. Onset can be predicted in vitro using isolated trachea from guinea pig or human.

In one aspect the present invention provides a compound of formula (I):

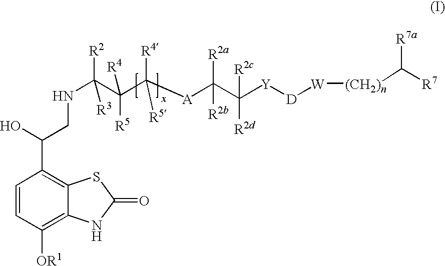

wherein
R¹ represents hydrogen;
each of R², R³, R⁴, R⁵, R^{4′} and R^{5′} independently represents hydrogen or C₁-C₆ alkyl;
x is 0 or 1;
A represents oxygen, sulphur, S(O) or S(O)₂;
D represents oxygen, sulphur or NR⁶;
W is a bond or CR^{6a}R^{6b};
n is an integer from 0 to 2;
R⁶ represents hydrogen, C₁-C₆ alkyl, C₁-C₆ alkoxycarbonyl or arylC₁-C₆alkyl;
Y is a bond, CR^{2e}R^{2f} or CR^{2g}R^{2h}CR^{2k}R^{2m};
R^{2a}, R^{2b}, R^{2c}, R^{2d}, R^{2e}, R^{2f}, R^{2g}, R^{2h}, R^{2k}, R^{2m}, R^{6a} and R^{6b} are all hydrogen;
R^{7a} is hydrogen;
R⁷ represents a 6- to 14-membered aromatic or heteroaromatic ring system optionally substituted by one or more substituents independently selected from halogen, trifluoromethyl, hydroxyl, carboxyl, C₁-C₆ alkyl (optionally substituted by at least one —NR¹⁰R¹¹), C₁-C₆ alkoxy (optionally substituted by at least one —NR¹²R¹³), C₁-C₆ alkoxycarbonyl, —NR¹⁴R¹⁵, C₁-C₆ alkylcarbonylamino, C₁-C₆ alkylsulphonylamino, phenylsulphonylamino, —C(O)NHR¹⁶, —SO₂NHR¹⁷, C₀-C₆ alkyl-R¹⁸, and a phenyl or 5- to 6-membered heteroaromatic ring (each of which may be optionally substituted by one or more substituents independently selected from halogen, trifluoromethyl, hydroxyl, C₁-C₆ alkyl, C₁-C₆ alkoxy and —NR²¹R²²);
R¹⁰, R¹¹, R¹², R¹³, R¹⁴ and R¹⁵ each independently represent hydrogen or C₁-C₆ alkyl;
R¹⁶ represents hydrogen, C₁-C₆ alkyl, phenyl-C₀-C₆ alkyl or C₂-C₆ alkylene-NR¹⁹R²⁰;
either R¹⁹ and R²⁰ each independently represent hydrogen or C₁-C₆ alkyl, or R¹⁹ and R²⁰ together with the nitrogen atom to which they are attached form a 4- to 6-membered saturated heterocyclic ring optionally comprising a further ring heteroatom selected from nitrogen and oxygen;
R¹⁷ represents hydrogen, C₁-C₆ alkyl, phenyl-C₀-C₆ alkyl or C₂-C₆ alkylene-NR²³R²⁴;
either R²³ and R²⁴ each independently represent hydrogen or C₁-C₆ alkyl, or R²³ and R²⁴ together with the nitrogen atom to which they are attached form a 4- to 6-membered saturated heterocyclic ring optionally comprising a further ring heteroatom selected from nitrogen and oxygen;
R¹⁸ represents a saturated, 5- or 6-membered nitrogen-containing ring; and
R²¹ and R²² each independently represent hydrogen or C₁-C₆ alkyl;
   or a pharmaceutically acceptable salt thereof.

In an embodiment of the invention, each of R², R³, R⁴, R⁵ and, if present, R^{4′} and R^{5′} independently represents hydrogen or C₁-C₆ (for example C₁-C₄ (such as C₁-C₂)) alkyl.

In another embodiment, each of R², R³, R⁴, R⁵ and, if present, R^{4′} and R^{5′} represents hydrogen.

The variable n is 0, 1 or 2 and is, for example, 1 or 2.

In a further embodiment A is oxygen, sulphur or S(O)₂.

In an embodiment of the invention, A represents oxygen.

In another embodiment of the invention, A represents sulphur.

In still another embodiment of the invention, A represents S(O)₂.

In an embodiment of the invention, D represents oxygen.

In another embodiment of the invention when W is a bond and n is 0 then D is not oxygen.

In a further embodiment of the invention D represents NR⁶.

In a still further embodiment of the invention D is NR⁶ and A is sulphur; wherein R⁶ is as defined above (for example R⁶ is hydrogen or C₁-C₆ alkyl).

In another embodiment of the invention D and A are not both oxygen.

In an embodiment of the invention, Y is a bond or CR^{2e}R^{2f}, wherein R^{2e} and R^{2f} are both hydrogen.

In another embodiment of the invention, n is 0 and W is CR^{6a}R^{6b}, wherein R^{6a} and R^{6b} are, independently, hydrogen or C₁₋₄ alkyl (for example methyl). For example R^{6a} and R^{6b} are both hydrogen.

In a further embodiment of the invention, n is 1 or 2 and W is CR^{6a}R^{6b}, wherein R^{6a} and R^{6b} are, independently, hydrogen or C₁₋₄ alkyl (for example methyl). For example R^{6a} and R^{6b} are both hydrogen.

In yet another embodiment R⁶ represents hydrogen; C₁-C₆, or C₁-C₄, or C₁-C₂ alkyl; C₁-C₆, or C₁-C₄, or C₁-C₂ alkoxycarbonyl; or arylC₁-C₆, or C₁-C₄, or C₁-C₂ alkyl.

In one embodiment, R⁶ represents hydrogen; C₁-C₄ or C₁-C₂ alkyl; C₁-C₄ or C₁-C₂ alkoxycarbonyl; phenylC₁-C₄ or C₁-C₂ alkyl; or naphthylC₁-C₄ or C₁-C₂ alkyl.

In another embodiment, R⁶ represents hydrogen, C₁-C₂ alkyl or C₁-C₄ alkoxycarbonyl.

In a further embodiment R⁷ represents a 5- to 14-membered (5-, 6-, 7-, 8-, 9-, 10-, 11-, 12-, 13- or 14-membered) {for example 6- to 14-membered (6-, 7-, 8-, 9-, 10-, 11-, 12-, 13- or 14-membered)} aromatic or heteroaromatic ring system optionally substituted by halogen (e.g. fluorine, chlorine, bromine or iodine), trifluoromethyl, hydroxyl, carboxyl, C₁-C₆, or C₁-C₄, or C₁-C₂ alkyl (optionally substituted by (for example by none, one or two) —NR¹⁰R¹¹), C₁-C₆, or C₁-C₄, or C₁-C₂ alkoxy (optionally substituted by (for example by none, one or two) —NR¹²R¹³), C₁-C₆, or C₁-C₄, or C₁-C₂ alkoxycarbonyl, —NR¹⁴R¹⁵, C₁-C₆, or C₁-C₄, or C₁-C₂ alkylcarbonylamino, C₁-C₆, or C₁-C₄, or C₁-C₂ alkylsulphonylamino, phenylsulphonylamino, —C(O)NHR¹⁶, —SO₂NHR¹⁷, C₀-C₆, or C₀-C₄, or C₀-C₂ alkyl-R¹⁸, or a phenyl or 5- to 6-membered heteroaromatic ring (each of which is optionally substituted by (for example by one, two, three or four) substituents independently selected from halogen (such as fluorine, chlorine, bromine or iodine), trifluoromethyl, hydroxyl, C₁-C₆, or C₁-C₄, or C₁-C₂ alkyl, C₁-C₆, or C₁-C₄, or C₁-C₂ alkoxy or —NR²¹R²²).

Thus the invention provides a compound of formula (I) wherein R⁷ represents a 5- to 14-membered (5-, 6-, 7-, 8-, 9-, 10-, 11-, 12-, 13- or 14-membered) {for example 6- to 14-membered (6-, 7-, 8-, 9-, 10-, 11-, 12-, 13- or 14-membered)} aromatic or heteroaromatic ring system optionally substituted by one or more (e.g. one, two, three or four) substituents independently selected from halogen (e.g. fluorine, chlorine, bromine or iodine), trifluoromethyl, hydroxyl, carboxyl, C₁-C₆, or C₁-C₄, or C₁-C₂ alkyl (optionally substituted by (for example by none, one or two) —NR¹⁰R¹¹), C₁-C₆, or C₁-C₄, or C₁-C₂ alkoxy (optionally substituted by (for example by none, one or two) —NR¹²R¹³), C₁-C₆, or C₁-C₄, or C₁-C₂ alkoxycarbonyl, —NR¹⁴R¹⁵, C₁-C₆, or C₁-C₄, or C₁-C₂ alkylcarbonylamino, C₁-C₆, or C₁-C₄, or C₁-C₂ alkylsulphonylamino, phenylsulphonylamino, —C(O)NHR¹⁶, —SO₂NHR¹⁷, C₀-C₆, or C₀-C₄, or C₀-C₂ alkyl-R¹⁸, and a phenyl or 5- to 6-membered heteroaromatic ring (each of which may be optionally substituted by one or more, e.g. one, two, three or four, substituents independently selected from halogen such as fluorine, chlorine, bromine or iodine, trifluoromethyl, hydroxyl, C₁-C₆, or C₁-C₄, or C₁-C₂ alkyl, C₁-C₆, or C₁-C₄, or C₁-C₂ alkoxy and —NR²¹R²²).

When R⁷ represents an optionally substituted 5- to 14-membered (for example 6- to 14-membered) heteroaromatic ring system, the ring system comprises from 1 to 4 ring heteroatoms independently selected from nitrogen, oxygen and sulphur. Similarly, if a substituent in R⁷ represents an optionally substituted 5- to 6-membered heteroaromatic ring, the ring comprises from 1 to 4 ring heteroatoms independently selected from nitrogen, oxygen and sulphur.

Examples of 5- to 14-membered (for example 6- to 14-membered) aromatic or heteroaromatic ring systems that may be used, which may be monocyclic or polycyclic (e.g. bicyclic or tricyclic) in which the two or more rings are fused, include one or more (in any combination) of phenyl, naphthyl, pyridinyl, pyridazinyl, pyrimidinyl, pyrazinyl, 1,3,5-triazinyl, 1,2,4-triazinyl, azepinyl, oxepinyl, thiepinyl, indenyl, benzofuranyl, isobenzofuranyl, benzothiophenyl, indolyl, isoindolyl, benzimidazolyl, indazolyl, benzisoxazolyl, benzoxazolyl, benzothiazolyl, quinolinyl, isoquinolinyl, quinazolinyl, quinoxalinyl and dibenzofuranyl. Preferred ring systems include phenyl and naphthyl.

Examples of 5- to 6-membered heteroaromatic rings include pyridinyl, triazolyl and tetrazolyl.

In an embodiment of the invention, R⁷ represents a 6- to 10-membered aromatic or heteroaromatic ring system optionally substituted by halogen (e.g. fluorine, chlorine, bromine or iodine), trifluoromethyl, hydroxyl, carboxyl, C₁-C₄ or C₁-C₂ alkyl (optionally substituted by (for example by one or two) —NR¹⁰R¹¹), C₁-C₄ or C₁-C₂ alkoxy (optionally substituted by (for example by one or two) —NR¹²R¹³), C₁-C₄ or C₁-C₂ alkoxycarbonyl, —NR¹⁴R¹⁵, C₁-C₄ or C₁-C₂ alkylcarbonylamino, C₁-C₄ or C₁-C₂ alkylsulphonylamino, phenylsulphonylamino, —C(O)NHR¹⁶, —SO₂NHR¹⁷, C₀-C₄ or C₀-C₂ alkyl-R⁸, phenyl or a 5- to 6-membered heteroaromatic ring.

Thus the invention provides a compound of formula (I) wherein R⁷ represents a 6- to 10-membered aromatic or heteroaromatic ring system optionally substituted by one or more (e.g. one, two, three or four) substituents independently selected from halogen (e.g. fluorine, chlorine, bromine or iodine), trifluoromethyl, hydroxyl, carboxyl, C₁-C₄ or C₁-C₂ alkyl (optionally substituted by at least one, e.g. one or two, —NR¹⁰R¹¹), C₁-C₄ or C₁-C₂ alkoxy (optionally substituted by at least one, e.g. one or two, —NR¹²R¹³), C₁-C₄ or C₁-C₂ alkoxycarbonyl, —NR¹⁴R¹⁵, C₁-C₄ or C₁-C₂ alkylcarbonylamino, C₁-C₄ or C₁-C₂ alkylsulphonylamino, phenylsulphonylamino, —C(O)NHR⁶, —SO₂NHR¹⁷, C₀-C₄ or C₀-C₂ alkyl-R⁸, phenyl and a 5- to 6-membered heteroaromatic ring.

In another embodiment, R⁷ represents a 6- to 10-membered aromatic ring system optionally substituted by one or two substituents independently selected from halogen (e.g. fluorine, chlorine, bromine or iodine), trifluoromethyl, hydroxyl, carboxyl, C₁-C₄ or C₁-C₂ alkyl (optionally substituted by (for example by one or two) —NR¹⁰R¹¹), C₁-C₄ or C₁-C₂ alkoxy (optionally substituted by (for example by one or two) —NR¹²R¹³), C₁-C₄ or C₁-C₂ alkoxycarbonyl, —NR¹⁴R¹⁵, C₁-C₄ or C₁-C₂ alkylcarbonylamino, C₁-C₄ or C₁-C₂ alkylsulphonylamino, phenylsulphonylamino, —C(O)NHR¹⁶, —SO₂NHR¹⁷, C₀-C₄ or C₀-C₂ alkyl-R¹⁸, phenyl or a 5- to 6-membered heteroaromatic ring.

Thus the invention provides a compound of formula (I) wherein R⁷ represents a 6- to 10-membered aromatic ring system optionally substituted by one or two substituents independently selected from halogen (e.g. fluorine, chlorine, bromine or iodine), trifluoromethyl, hydroxyl, carboxyl, C₁-C₄ or C₁-C₂ alkyl (optionally substituted by at least one, e.g. one or two, —NR¹⁰R¹¹), C₁-C₄ or C₁-C₂ alkoxy (optionally substituted by at least one, e.g. one or two, —NR¹²R¹³), C₁-C₄ or C₁-C₂ alkoxycarbonyl, —NR¹⁴R¹⁵, C₁-C₄ or C₁-C₂ alkylcarbonylamino, C₁-C₄ or C₁-C₂ alkylsulphonylamino, phenylsulphonylamino, —C(O)NHR¹⁶, —SO₂NHR¹⁷, C₀-C₄ or C₀-C₂ alkyl-R¹⁸, phenyl and a 5- to 6-membered heteroaromatic ring.

In a further embodiment, R⁷ represents a 6- to 10-membered aromatic ring system optionally substituted by (for example by one, two, three or four) halogen atoms.

Thus the invention provides a compound of formula (I) wherein R⁷ represents a 6- to 10-membered aromatic ring system optionally substituted by one or more (e.g. one, two, three or four) halogen atoms.

In another embodiment R¹⁰, R¹¹, R¹², R¹³, R¹⁴ and R¹⁵ each independently represent hydrogen or C₁-C₆, or C₁-C₄, or C₁-C₂ alkyl. It should be understood that if there is more than one group —NR¹⁰R¹¹, the groups may be the same as, or different from, one another. Similar comments apply if there is more than one group —NR¹²R¹³.

In a further embodiment R¹⁶ represents hydrogen; C₁-C₆, or C₁-C₄, or C₁-C₂ alkyl; phenyl-C₀-C₆, or C₀-C₄, or C₀-C₂ alkyl (e.g. phenyl or benzyl); or C₂-C₆ or C₂-C₄ alkylene-NR¹⁹R²⁰ and either R¹⁹ and R²⁰ each independently represent hydrogen or C₁-C₆, or C₁-C₄, or C₁-C₂ alkyl, or R¹⁹ and R²⁰ together with the nitrogen atom to which they are attached form a 4- to 6-membered saturated heterocyclic ring optionally comprising a further ring heteroatom selected from nitrogen and oxygen such as azetidinyl, pyrrolidinyl, piperidinyl, piperazinyl or morpholinyl.

In a still further embodiment R¹⁷ represents hydrogen; C₁-C₆, or C₁-C₄, or C₁-C₂ alkyl; phenyl-C₀-C₆, or C₀-C₄, or C₀-C₂ alkyl (e.g. phenyl or benzyl); or C₂-C₆ or C₂-C₄ alkylene-NR²³R²⁴ and either R²³ and R²⁴ each independently represent hydrogen or C₁-C₆, or C₁-C₄, or C₁-C₂ alkyl, or R²³ and R²⁴ together with the nitrogen atom to which they are attached form a 4- to 6-membered saturated heterocyclic ring optionally comprising a further ring heteroatom selected from nitrogen and oxygen such as azetidinyl, pyrrolidinyl, piperidinyl, piperazinyl or morpholinyl.

In another embodiment R^{1g} represents a saturated, 5- or 6-membered nitrogen-containing ring, e.g. a ring containing one or two ring nitrogen atoms such as hydantoin.

In yet another embodiment R²¹ and R²² each independently represent hydrogen or C₁-C₆, or C₁-C₄, or C₁-C₂ alkyl.

In a further embodiment R^{7a} is hydrogen or C₁-C₆ alkyl.

In another embodiment R^{7a} is NHR^{7b} wherein R^{7b} is hydrogen, C₁-C₄ alkyl or C₁-C₆ alkoxycarbonyl (for example hydrogen or C₁-C₆ alkoxycarbonyl).

In a further embodiment of the invention D is oxygen or sulphur (for example oxygen) and R^{7a} is NHR^{7b}, wherein R^{7b} is as defined above (for example it is hydrogen, C₁-C₄ alkyl or C₁-C₆ alkoxycarbonyl).

In an embodiment the invention provides a compound of formula (I) wherein:
x is 0 or 1;
A represents oxygen, sulphur or S(O)₂;
D represents oxygen or NR⁶;
Y is a bond or CR^{2e}R^{2f};
W is CR^{6a}R^{6b};
n is 0;
R¹, R², R³, R⁴, R⁵, R^{4′}, R^{5′}, R^{2a}, R^{2b}, R^{2c}, R^{2d}, R^{2e}, R^{2f}, R^{6a}, R^{6b} and R^{7a} are all hydrogen;
R⁶ represents hydrogen, methyl or C₄ alkoxycarbonyl;
R⁷ represents a 6- to 10-membered aromatic ring system optionally substituted by one or more halogen atoms; for example said compound of formula (I) can be in free-base form or in the form of a pharmaceutically acceptable salt.

In a further embodiment the invention provides a compound of formula (I) wherein:
x is 0 or 1;
A represents oxygen, sulphur or S(O)₂;
D represents oxygen or NR⁶;
Y is a bond or CH₂;
W is CR^{6a}R^{6b};
n is 0;

R¹, R², R³, R⁴, R⁵, R^{4′}, R^{5′}, R^{2a}, R^{2b}, R^{2c} and R^{2d} are all hydrogen;
R⁶ represents hydrogen, C₁-C₄ alkyl (for example methyl) or C₄ alkoxycarbonyl (such as tert-butoxycarbonyl);
   R^{6a} and R^{6b} are, independently, hydrogen or C₁-C₄ alkyl (for example methyl);
R⁷ represents a 6- to 10-membered aromatic ring system (such as phenyl or naphthyl) optionally substituted by halogen (for example chlorine or bromine), C₁-C₄ alkyl (for example ethyl), C₁-C₄ alkoxy (for example ethoxy) or CF₃; and,
   R^{7a} is hydrogen, C₁-C₄ alkyl (for example methyl), NH(C₄ alkoxycarbonyl) (such as NH(tert-butoxycarbonyl)) or NH₂;
   or a pharmaceutically acceptable salt thereof (such as a hydrochloride, a hydrobromide or a trifluoroacetate; for example a hydrochloride or a hydrobromide).

In yet another embodiment the invention provides a compound of formula (I) wherein:
x is 0 or 1;
A represents oxygen, sulphur or S(O)₂;
D represents or NR⁶;
Y is a bond or CH₂;
W is CR^{6a}R^{6b};
n is 0;
R¹, R², R³, R⁴, R⁵, R^{4′}, R^{5′}, R^{2a}, R^{2b}, R^{2c} and R^{2d} are all hydrogen;
R⁶ represents hydrogen, C₁-C₄ alkyl (for example methyl) or C₄ alkoxycarbonyl (such as tert-butoxycarbonyl);
   R^{6a} and R^{6b} are, independently, hydrogen or C₁-C₄ alkyl (for example methyl);
R⁷ represents a 6- to 10-membered aromatic ring system (such as phenyl or naphthyl) optionally substituted by halogen (for example chlorine or bromine), C₁-C₄ alkyl (for example ethyl), C₁-C₄ alkoxy (for example ethoxy) or CF₃; and,
   R^{7a} is hydrogen, C₁-C₄ alkyl (for example methyl), NH(C₄ alkoxycarbonyl) (such as NH(tert-butoxycarbonyl)) or NH₂;
   or a pharmaceutically acceptable salt thereof (such as a hydrochloride, a hydrobromide or a trifluoroacetate; for example a hydrochloride or a hydrobromide).

In a further embodiment the invention provides a compound of formula (I) wherein:
x is 0 or 1;
A represents oxygen, sulphur or S(O)₂;
D represents oxygen or NR⁶, (for example oxygen)
Y is a bond or CH₂;
W is CR^{6a}R^{6b};
n is 0;
R¹, R², R³, R⁴, R⁵, R^{4′}, R^{5′}, R^{2a}, R^{2b}, R^{2c} and R^{2d} are all hydrogen;
R⁶ represents hydrogen, C₁-C₄ alkyl (for example methyl) or C₄ alkoxycarbonyl (such as tert-butoxycarbonyl);
   R^{6a} and R^{6b} are, independently, hydrogen or C₁-C₄ alkyl (for example methyl);
R⁷ represents a 6- to 10-membered aromatic ring system (such as phenyl or naphthyl) optionally substituted by halogen (for example chlorine or bromine), C₁-C₄ alkyl (for example ethyl), C₁-C₄ alkoxy (for example ethoxy) or CF₃; and,
   R⁷¹ is NH(C₄ alkoxycarbonyl) (such as NH(tert-butoxycarbonyl)), NH(C₁-C₄ alkyl) or NH₂;
   or a pharmaceutically acceptable salt thereof (such as a hydrochloride, a hydrobromide or a trifluoroacetate; for example a hydrochloride or a hydrobromide).

In another embodiment of the invention there is provided a compound:
4-Hydroxy-7-((1R)-1-hydroxy-2-{[3-({2-[2-(1-naphthyl)ethoxy]ethyl}thio)propyl]-amino}ethyl)-1,3-benzothiazol-2(3H)-one,
4-Hydroxy-7-((1R)-1-hydroxy-2-{[3-({2-[2-(1-naphthyl)ethoxy]ethyl}sulfonyl)propyl]amino}ethyl)-1,3-benzothiazol-2(3H)-one,
4-Hydroxy-7-[(1R)-1-hydroxy-2-({3-[2-(2-phenylethoxy)ethoxy]propyl}amino)ethyl]-1,3-benzothiazol-2(3H)-one,
4-Hydroxy-7-((1R)-1-hydroxy-2-{[2-({2-[2-(1-naphthyl)ethoxy]ethyl}thio)ethyl]-amino}ethyl)-1,3-benzothiazol-2(3H)-one,
7-((1R)-2-{[3-({3-[2-(4-Bromophenyl)ethoxy]propyl}thio)propyl]amino}-1-hydroxyethyl)-4-hydroxy-1,3-benzothiazol-2(3H)-one,
4-Hydroxy-7-{(1R)-1-hydroxy-2-[(3-{[3-(2-phenylethoxy)propyl]thio}propyl)amino]ethyl}-1,3-benzothiazol-2(3H)-one,
4-Hydroxy-7-{(1R)-1-hydroxy-2-[(3-{2-[2-(1-naphthyl)ethoxy]ethoxy}propyl)amino]ethyl}-1,3-benzothiazol-2(3H)-one,
4-Hydroxy-7-{(1R)-1-hydroxy-2-[(2-{[3-(2-phenylethoxy)propyl]thio}-ethyl)amino]ethyl}-1,3-benzothiazol-2(3H)-one,
4-Hydroxy-7-{(1R)-1-hydroxy-2-[(3-{[2-(2-phenylethoxy)ethyl]thio}-propyl)amino]ethyl}-1,3-benzothiazol-2(3H)-one,
tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}(2-phenylethyl)carbamate,
4-Hydroxy-7-((1R)-1-hydroxy-2-{[2-({3-[(2-phenylethyl)amino]propyl}thio)ethyl]amino}ethyl)-1,3-benzothiazol-2(3H)-one,
4-Hydroxy-7-((1R)-1-hydroxy-2-{[2-({3-[methyl(2-phenylethyl)amino]propyl}-thio)ethyl]amino}ethyl)-1,3-benzothiazol-2(3H)-one,
tert-Butyl [2-(4-ethylphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(4-Ethylphenyl)ethyl]amino}propyl)thio]ethyl}amino)-1-hydroxyethyl]-4-hydroxy-1,3-benzothiazol-2(3H)-one,
tert-Butyl [2-(4-ethoxyphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(4-Ethoxyphenyl)ethyl]amino}propyl)thio]ethyl}amino)-1-hydroxyethyl]-4-hydroxy-1,3-benzothiazol-2(3H)-one,
tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}{2-[3-(trifluoromethyl)phenyl]ethyl}carbamate,
4-Hydroxy-7-{(1R)-1-hydroxy-2-[(2-{[3-({2-[3-(trifluoromethyl)phenyl]ethyl}amino)propyl]thio}ethyl)amino]ethyl}-1,3-benzothiazol-2(3H)-one,
tert-Butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one,
tert-Butyl ((1S)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate,
7-((1R)-2-{[2-({3-[(2S)-2-Amino-2-phenylethoxy]propyl}thio)ethyl]amino}-1-hydroxyethyl)-4-hydroxy-1,3-benzothiazol-2(3H)-one,
tert-Butyl ((1R)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate,
7-((1R)-2-{[2-({3-[(2R)-2-Amino-2-phenylethoxy]propyl}thio)ethyl]amino}-1-hydroxyethyl)-4-hydroxy-1,3-benzothiazol-2(3H)-one,
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one,
tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2R)-2-phenylpropyl]carbamate,
4-Hydroxy-7-[(1R)-1-hydroxy-2-({3-[(2-{[(2R)-2-phenylpropyl]amino}ethyl)thio]propyl}amino)ethyl]-1,3-benzothiazol-2(3H)-one,
tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2S)-2-phenylpropyl]carbamate,
4-Hydroxy-7-[(1R)-1-hydroxy-2-({3-[(2-{[(2S)-2-phenylpropyl]amino}ethyl)thio]propyl}amino)ethyl]-1,3-benzothiazol-2(3H)-one,
tert-Butyl [2-(2-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate,
7-[(1R)-2-({3-[(2-{[2-(2-Chlorophenyl)ethyl]amino}ethyl)thio]propyl}amino)-1-hydroxyethyl]-4-hydroxy-1,3-benzothiazol-2(3H)-one,
tert-Butyl [2-(3-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate,
7-[(1R)-2-({3-[(2-{[2-(3-Chlorophenyl)ethyl]amino}ethyl)thio]propyl}amino)-1-hydroxyethyl]-4-hydroxy-1,3-benzothiazol-2(3H)-one,
tert-Butyl [2-(2,3-dichlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate,
7-[(1R)-2-({2-[(3-{[2-(2,3-Dichlorophenyl)ethyl]amino}propyl)thio]ethyl}amino)-1-hydroxyethyl]-4-hydroxy-1,3-benzothiazol-2(3H)-one,
7-((1R)-2-{[2-(3-{[2-(3-Chlorophenyl)ethyl]amino}propoxy)ethyl]amino}-1-hydroxyethyl)-4-hydroxy-1,3-benzothiazol-2(3H)-one,
tert-Butyl [2-(2,3-dichlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(2,3-Dichlorophenyl)ethyl]amino}propyl)thio]ethyl}amino)-1-hydroxyethyl]-4-hydroxy-1,3-benzothiazol-2(3H)-one,
tert-Butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(3-Chlorophenyl)ethyl]amino}propyl)thio]ethyl}amino)-1-hydroxyethyl]-4-hydroxy-1,3-benzothiazol-2(3H)-one,
tert-Butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)sulfonyl]propyl}carbamate,
7-[(1R)-2-({2-[(3-{[2-(3-Chlorophenyl)ethyl]amino}propyl)sulfonyl]ethyl}amino)-1-hydroxyethyl]-4-hydroxy-1,3-benzothiazol-2(3H)-one,
(+/−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
(+/−)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one,
(R)-(+)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
(R)-(+)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one,
(S)-(−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
(S)-(−)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one,
tert-Butyl [2-methyl-2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate, or
7-[(1R)-2-({2-[(3-{[2-methyl-2-(phenyl)propyl]amino}propyl)thio]ethyl}amino)-1-hydroxyethyl]-4-hydroxy-1,3-benzothiazol-2(3H)-one,
   or a pharmaceutically acceptable salt thereof.

In a further embodiment the invention provides a compound:
4-Hydroxy-7-((1R)-1-hydroxy-2-{[3-({2-[2-(1-naphthyl)ethoxy]ethyl}thio)propyl]-amino}ethyl)-1,3-benzothiazol-2(3H)-one trifluoroacetate,
4-Hydroxy-7-((1R)-1-hydroxy-2-{[3-({2-[2-(1-naphthyl)ethoxy]ethyl}sulfonyl)propyl]amino}ethyl)-1,3-benzothiazol-2(3H)-one trifluoroacetate,
4-Hydroxy-7-[(1R)-1-hydroxy-2-({3-[2-(2-phenylethoxy)ethoxy]propyl}amino)ethyl]-1,3-benzothiazol-2(3H)-one trifluoroacetate,
4-Hydroxy-7-((1R)-1-hydroxy-2-{[2-({2-[2-(1-naphthyl)ethoxy]ethyl}thio)ethyl]-amino}ethyl)-1,3-benzothiazol-2(3H)-one hydrochloride,
7-((1R)-2-{[3-({3-[2-(4-Bromophenyl)ethoxy]propyl}thio)propyl]amino}-1-hydroxyethyl)-4-hydroxy-1,3-benzothiazol-2(3H)-one hydrochloride,
4-Hydroxy-7-{(1R)-1-hydroxy-2-[(3-{[3-(2-phenylethoxy)propyl]thio}propyl)amino]ethyl}-1,3-benzothiazol-2(3H)-one hydrochloride,
4-Hydroxy-7-{(1R)-1-hydroxy-2-[(3-{2-[2-(1-naphthyl)ethoxy]ethoxy}propyl)amino]ethyl}-1,3-benzothiazol-2(3H)-one,
4-Hydroxy-7-{(1R)-1-hydroxy-2-[(2-{[3-(2-phenylethoxy)propyl]thio}-ethyl)amino]ethyl}-1,3-benzothiazol-2(3H)-one hydrochloride,
4-Hydroxy-7-{(1R)-1-hydroxy-2-[(3-{[2-(2-phenylethoxy)ethyl]thio}-propyl)amino]ethyl}-1,3-benzothiazol-2(3H)-one hydrochloride,
tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}(2-phenylethyl)carbamate,
4-Hydroxy-7-((1R)-1-hydroxy-2-{[2-({3-[(2-phenylethyl)amino]propyl}thio)ethyl]amino}ethyl)-1,3-benzothiazol-2(3H)-one hydrochloride,
4-Hydroxy-7-((1R)-1-hydroxy-2-{[2-({3-[methyl(2-phenylethyl)amino]propyl}-thio)ethyl]amino}ethyl)-1,3-benzothiazol-2(3H)-one trifluoroacetate,
tert-Butyl [2-(4-ethylphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(4-Ethylphenyl)ethyl]amino}propyl)thio]ethyl}amino)-1-hydroxyethyl]-4-hydroxy-1,3-benzothiazol-2(3H)-one ditrifluoroacetate,
5 tert-Butyl [2-(4-ethoxyphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(4-Ethoxyphenyl)ethyl]amino}propyl)thio]ethyl}amino)-1-hydroxyethyl]-4-hydroxy-1,3-benzothiazol-2(3H)-one ditrifluoroacetate,
tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}{2-[3-(trifluoromethyl)phenyl]ethyl}carbamate,
4-Hydroxy-7-{(1R)-1-hydroxy-2-[(2-{[3-({2-[3-(trifluoromethyl)phenyl]ethyl}amino)propyl]thio}ethyl)amino]ethyl}-1,3-benzothiazol-2(3H)-one ditrifluoroacetate,
tert-Butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one ditrifluoroacetate,
tert-Butyl ((1S)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate,
7-((1R)-2-{[2-({3-[(2S)-2-Amino-2-phenylethoxy]propyl}thio)ethyl]amino}-1-hydroxyethyl)-4-hydroxy-1,3-benzothiazol-2(3H)-one ditrifluoroacetate,
tert-Butyl ((1R)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate,
7-((1R)-2-{[2-({3-[(2R)-2-Amino-2-phenylethoxy]propyl}thio)ethyl]amino}-1-hydroxyethyl)-4-hydroxy-1,3-benzothiazol-2(3H)-one ditrifluoroacetate,
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide,
tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2R)-2-phenylpropyl]carbamate,
4-Hydroxy-7-[(1R)-1-hydroxy-2-({3-[(2-{[(2R)-2-phenylpropyl]amino}ethyl)thio]propyl}amino)ethyl]-1,3-benzothiazol-2(3H)-one ditrifluoroacetate,
tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2S)-2-phenylpropyl]carbamate,
4-Hydroxy-7-[(1R)-1-hydroxy-2-({3-[(2-{[(2S)-2-phenylpropyl]amino}ethyl)thio]propyl}amino)ethyl]-1,3-benzothiazol-2(3H)-one ditrifluoroacetate,
tert-Butyl [2-(2-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate,
7-[(1R)-2-({3-[(2-{[2-(2-Chlorophenyl)ethyl]amino}ethyl)thio]propyl}amino)-1-hydroxyethyl]-4-hydroxy-1,3-benzothiazol-2(3H)-one dihydrobromide,
tert-Butyl [2-(3-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate,
7-[(1R)-2-({3-[(2-{[2-(3-Chlorophenyl)ethyl]amino}ethyl)thio]propyl}amino)-1-hydroxyethyl]-4-hydroxy-1,3-benzothiazol-2(3H)-one dihydrobromide,
tert-Butyl [2-(2,3-dichlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate,
7-[(1R)-2-({2-[(3-{[2-(2,3-Dichlorophenyl)ethyl]amino}propyl)thio]ethyl}amino)-1-hydroxyethyl]-4-hydroxy-1,3-benzothiazol-2(3H)-one dihydrobromide,
7-((1R)-2-{[2-(3-{[2-(3-Chlorophenyl)ethyl]amino}propoxy)ethyl]amino}-1-hydroxyethyl)-4-hydroxy-1,3-benzothiazol-2(3H)-one,
tert-Butyl [2-(2,3-dichlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(2,3-Dichlorophenyl)ethyl]amino}propyl)thio]ethyl}amino)-1-hydroxyethyl]-4-hydroxy-1,3-benzothiazol-2(3H)-one dihydrochloride,
tert-Butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(3-Chlorophenyl)ethyl]amino}propyl)thio]ethyl}amino)-1-hydroxyethyl]-4-hydroxy-1,3-benzothiazol-2(3H)-one dihydrochloride,
tert-Butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)sulfonyl]propyl}carbamate,
7-[(1R)-2-({2-[(3-{[2-(3-Chlorophenyl)ethyl]amino}propyl)sulfonyl]ethyl}amino)-1-hydroxyethyl]-4-hydroxy-1,3-benzothiazol-2(3H)-one dihydrochloride,
(+/−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
(+/−)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one dihydrochloride,
(R)-(+)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
(R)-(+)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one dihydrochloride,
(S)-(−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
(S)-(−)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one dihydrochloride,
tert-Butyl [2-methyl-2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate, or
7-[(1R)-2-({2-[(3-{[2-methyl-2-(phenyl)propyl]amino}propyl)thio]ethyl}amino)-1-hydroxyethyl]-4-hydroxy-1,3-benzothiazol-2(3H)-one dihydrochloride.

Each exemplified compound represents an independent and particular aspect of the invention.

The present invention further provides a process for the preparation of a compound of formula (I) or a pharmaceutically acceptable salt thereof as defined above which comprises,

(a) reacting a compound of formula (II)

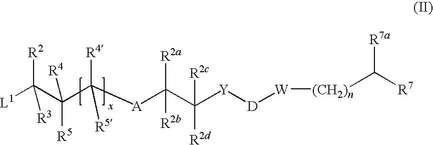

wherein L¹ represents a leaving group (e.g. chlorine, bromine, iodine, methanesulfonate or para-toluenesulfonate) and the other variables are as defined in formula (I), with a compound of formula (III) or a suitable salt thereof (e.g. hydrobromide or hydrochloride salt)

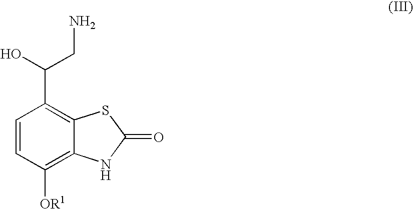

wherein R¹ is as defined in formula (I), in the presence of a base (e.g. potassium carbonate, triethylamine or diisopropylethylamine); or
(b) when R² and R³ each represent hydrogen, reacting a compound of formula (IV)

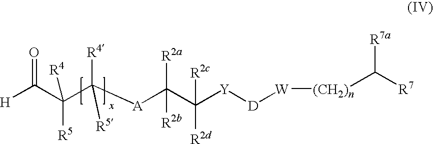

wherein the variables are as defined in formula (I), with a compound of formula (III) or a suitable salt thereof as defined in (a) above in the presence of a suitable reducing agent (e.g. sodium cyanoborohydride, sodium triacetoxyborohydride, or hydrogen in the presence of a palladium on carbon or palladium oxide catalyst); or
(c) when R² and R³ each represent hydrogen, contacting a compound of formula (V)

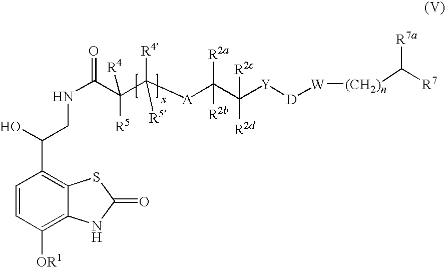

wherein the variables are as defined in formula (I) with a suitable reducing agent (e.g. lithium aluminium hydride or borane tetrahydrofuran complex);
and optionally after (a), (b) or (c) carrying out one or more of the following:
converting the compound obtained to a further compound of the invention
forming a pharmaceutically acceptable salt of the compound.

In process (a), the reaction may conveniently be carried out in an organic solvent such as N,N-dimethylformamide, ethanol, n-butanol or dimethyl sulfoxide, at a temperature, for example, in the range from 25 to 100° C.

In process (b), the reaction may conveniently be carried out in an organic solvent such as methanol, ethanol, dichloromethane, or N,N-dimethylformamide containing up to 10% w of water.

In process (c), the reaction may conveniently be carried out in an organic solvent such as tetrahydrofuran or diethyl ether, at a temperature, for example, in the range from 0 to 60° C.

Compounds of formula (II) in which L¹ represents, for example, bromine may be prepared by reacting a compound of formula (X)

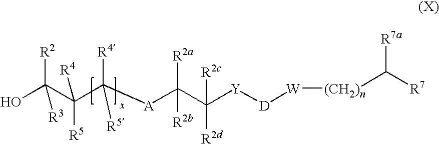

wherein the variables are as defined in formula (II) with N-bromosuccinimide and triphenylphosphine in a solvent, for example, dichloromethane at a temperature, for example, in the range from −10 to 20° C.

Compounds of formula (X) in which A represents oxygen or sulphur may be prepared by reacting a compound of formula (XI)

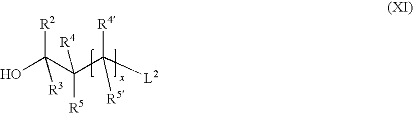

wherein L² represents a leaving group (e.g. chlorine, bromine, iodine, methanesulfonate or para-toluenesulfonate) and x, R², R³, R⁴, R⁵, R^{4′} and R^{5′} are as defined in formula (X), with a compound of formula (XII)

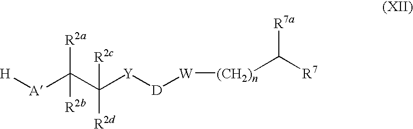

wherein A′ represents oxygen or sulphur and the other variables are as defined in formula (X), in the presence of a suitable base, for example, potassium carbonate, triethylamine, sodium hydride or diisopropylethylamine in an organic solvent, for example, tetrahydrofuran or dimethyl sulphoxide at a temperature, for example, in the range from 0 to 50° C.

Compounds of formula (X) in which A represents sulphinyl or sulphonyl may be prepared by oxidising a corresponding compound of formula (X) in which A represents sulphur using, for example, meta-chloroperoxybenzoic acid or hydrogen peroxide, in an organic solvent, for example, methanol, ethanol or dichloromethane at a temperature, for example, in the range from 0 to 50° C.

Compounds of formula (III) may be prepared by reducing a compound of formula (XIII)

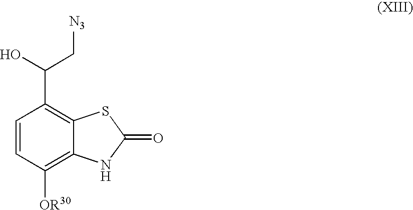

in which R³⁰ represents hydrogen or benzyl, with a suitable reducing agent, for example, hydrogen in the presence of a suitable catalyst, for example, 5-10% w palladium on carbon or platininum oxide at a pressure of 1-5 atmospheres. The reaction is conveniently carried out in an organic solvent such as ethanol, methanol, ethyl acetate or tetrahydrofuran.

Compounds of formula (XIII) may be prepared by reacting a compound of formula (XIV)

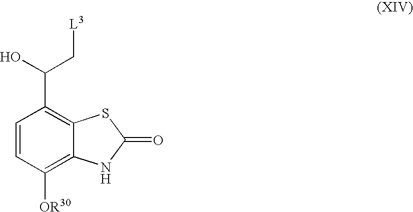

wherein L³ represents a leaving group (e.g. bromine, iodine, methanesulfonate or para-toluenesulfonate) and R³⁰ is as defined in formula (XIII), with sodium azide in the presence of, for example, sodium iodide, lithium iodide or tetrabutyl ammonium iodide. The reaction is conveniently carried out in an organic solvent, for example dimethyl sulphoxide or N,N-dimethylformamide, at a temperature, for example, in the range from 10 to 80° C., specifically from 50 to 70° C.

Compounds of formula (III) in which R¹ is hydrogen may be prepared by reacting a corresponding compound in which R¹ is replaced by benzyl with a suitable reducing agent, for example, hydrogen in the presence of a suitable catalyst, for example, 5-10% w palladium on carbon at a pressure of 1-5 atmospheres. The reaction is conveniently carried out in an organic solvent such as ethanol or methanol containing 5-10% w concentrated hydrochloric acid.

Compounds of formula (IV) may be prepared by oxidising a compound of formula (XV)

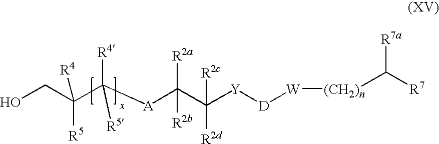

wherein the variables are as defined in formula (IV), with an oxidising agent, for example pyridinium chlorochromate or Dess-Martin periodinane in an organic solvent, for example, dichloromethane at a temperature, for example, of 25° C. Other oxidative procedures may also be employed as known to persons skilled in the art, for example, the Swern oxidation which is outlined in Synthesis, 1981, 3, 165.

Compounds of formula (XV) may be prepared as described above for the compounds of formula (X).

Compounds of formula (V) may be prepared by reacting a compound of formula (XVI)

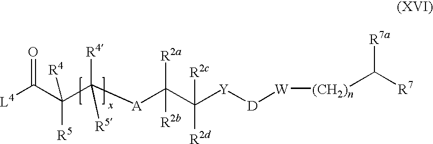

wherein L⁴ represents a leaving group (e.g. chlorine or hydroxyl) and the other variables are as defined in formula (V), with a compound of formula (III) or a suitable salt thereof as defined above.

When L⁴ represents chlorine, the reaction is conveniently carried out in the presence of a base, for example, triethylamine or diisopropylethylamine in an organic solvent, for example, dichloromethane at a temperature, for example, in the range from 0 to 25° C.

When L⁴ represents hydroxyl, the reaction is conveniently carried out in the presence of an activating reagent, for example, carbonyldiimidazole or O-(7-azabenzotriazol-1-yl)-N,N,N′,N′-tetramethyluroniumhexafluorophosphate (HATU), in an organic solvent, for example, N,N-dimethylformamide or dichloromethane, at a temperature, for example in the range from 0 to 60° C.

Compounds of formula (XVI) in which L⁴ represents, for example, hydroxyl may be prepared by processes analogous to those described for the preparation of the compounds of formula (X).

Routes to compounds wherein A is oxygen or sulphur are shown after the Examples. These routes can be adapted using alternative intermediates to prepare other compounds of formula (I).

Compounds of formulae (XI), (XII), (XIV) and (XVI) are either commercially available, are known in the literature or may be prepared using known techniques.

The present invention further relates to novel intermediate compounds, for example, compounds of formula (III′)

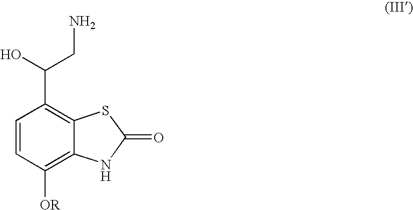

wherein R represents hydrogen or benzyl, and the compound

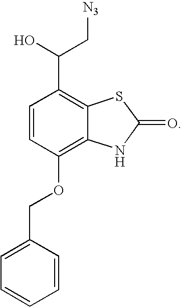

Compounds of formula (I) can be converted into further compounds of formula (I) using standard procedures. For example, a compound of formula (I) in which A represents sulphur can be converted to a corresponding compound of formula (I) in which A represents sulphonyl by an oxidation reaction using, for example, meta-chloroperoxybenzoic acid or hydrogen peroxide, in an organic solvent, for example, methanol, ethanol or dichloromethane at a temperature, for example, in the range from 0 to 50° C.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, trifluoroacetate, sulphate, phosphate, acetate, fumarate, maleate, tartrate, lactate, citrate, pyruvate, succinate, oxalate, methanesulphonate or p-toluenesulphonate.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

In a further embodiment the present invention provides a compound of formula (I) having (R) absolute configuration at the asterisked (*) carbon below.

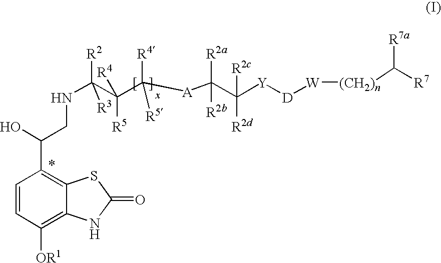

A compound of formula (I), or a pharmaceutically acceptable salt thereof, can exist a solvate (such as a hydrate). The present invention covers such solvates.

The compounds of formula (I) and their pharmaceutically acceptable salts can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Pagefs disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dennatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopenic purpura, eosinophilic fascitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In another aspect the invention provides the use of a compound of formula (I) or a pharmaceutically acceptable salt thereof as hereinbefore defined in the manufacture of a medicament for the treatment of human diseases or conditions in which modulation of P2 adrenoreceptor activity is beneficial.

In a still further aspect the present invention provides a method of treating, or reducing the risk of, a disease or condition in which modulation of P2 adrenoreceptor activity is beneficial which comprises administering to a patient in need thereof a therapeutically effective amount of a compound of formula (I) or a pharmaceutically acceptable salt thereof as hereinbefore defined.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention still further provides a method of treating, or reducing the risk of, an inflammatory disease or condition (including a reversible obstructive airways disease or condition) which comprises administering to a patient in need thereof a therapeutically effective amount of a compound of formula (I) or a pharmaceutically acceptable salt thereof as hereinbefore defined.

In particular, the compounds of this invention may be used in the treatment of adult respiratory distress syndrome (ARDS), pulmonary emphysema, bronchitis, bronchiectasis, chronic obstructive pulmonary disease (COPD), asthma or rhinitis.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The invention therefore further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXC2, CXCR3, CXCR4 and CXCR5 (for the C-X-C family) and CX₃CR1 for the C-X₃-C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof. A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2×7; (xxvii) inhibitor of transcription factor activation such as NFkB, API, or STATS; or (xxviii) a non-steroidal glucocorticoid receptor agonist.

Where such a combination is to be administered by inhalation, then the one or more agents in addition to a compound of formula (I), or a pharmaceutically acceptable salt thereof, can be selected from the list comprising:
a PDE4 inhibitor including an inhibitor of the isoform PDE4D;
a glucocorticoid receptor agonist, {for example a non-steroidal glucocorticoid receptor agonist, or steroidal glucocorticoid receptor agonist (such as budesonide)};
a muscarinic receptor antagonist (for example a M1, M2 or M3 antagonist, such as a selective M3 antagonist) such as ipratropium bromide, tiotropium bromide, oxitropium bromide, pirenzepine, telenzepine or a glycopyrromium bromide (such as R,R-glycopyrronium bromide or a mixture of R,S- and S,R-glycopyrronium bromide);
a modulator of chemokine receptor function (such as a CCR1 receptor antagonist); or,
an inhibitor of p38 kinase function.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will now be further explained by reference to the following illustrative examples.

### GENERAL METHODS

¹H NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Column chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 8 min, 95% B 1 min.

Analytical chromatography was run on a Symmetry C₁₈-column, 2.1×30 mm with 3.5 μm particle size, with acetonitrile/water/0.1% trifluoroacetic acid as mobile phase in a gradient from 5% to 95% acetonitrile over 8 minutes at a flow of 0.7 ml/min.

The abbreviations or terms used in the examples have the following meanings:

HPLC: High performance liquid chromatography

### EXAMPLE 1

### 4-Hydroxy-7-((1R)-1-hydroxy-2-{[3-({2-[2-(1-naphthyl)ethoxy]ethyl}thio)propyl]-amino}ethyl)-1,3-benzothiazol-2(3H)-one trifluoroacetate

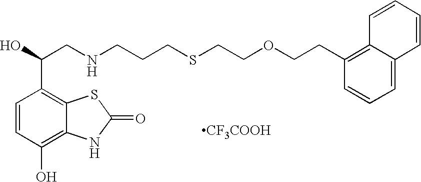

### a) 3-[2-(2-Naphthalen-1-yl-ethoxy)-ethylsulfanyl]-propan-1-ol

A mixture of 1-(2-vinyloxy-ethyl)-naphthalene (prepared as described in WO 97/23470) (2.04 g), 3-mercapto-1-propanol (0.95 g) and 2,2′-azobisisobutyronitrile (0.05 g) was heated at 60° C. for 3 hours. The mixture was then cooled to room temperature and purified by flash chromatography on silica gel eluting with 40% ethyl acetate in iso-hexane to yield the sub-titled compound (2.4 g).

¹H NMR (400 MHz, D₆-DMSO) δ 8.08 (d, 1H), 7.92 (d, 1H), 7.80-7.78 (m, 1H), 7.57-7.49 (m, 2H), 7.45-7.41 (m, 2H), 4.45 (t, 1H), 3.73 (t, 2H), 3.57 (t, 2H), 3.44 (q, 2H), 3.31-3.28 (m, 2H), 2.62 (t, 2H), 2.54 (t, 2H), 1.66-1.60 (m, 2H).

### b) 3-[2-(2-Naphthalen-1-yl-ethoxy)-ethylsulfanyl]-propionaldehyde

A solution of oxalyl chloride (482 mg) in dichloromethane (15 mL) stirred at −78° C. under nitrogen was treated dropwise with a solution of dimethyl sulfoxide (593 mg) in dichloromethane (2 mL). The mixture was stirred at −78° C. for 10 minutes and then treated dropwise with a solution of 3-[2-(2-naphthalen-1-yl-ethoxy)-ethylsulfanyl]-propan-1-ol (Example 1a), 1.0 g) in dichloromethane (3 mL). After a further 15 minutes at −78° C. triethylamine (1.74 g) was added dropwise, the mixture was then allowed to warm to room temperature over 1 hour. The reaction mixture was quenched by addition of saturated aqueous ammonium chloride solution (10 mL), the organic phase was washed with saturated aqueous sodium bicarbonate solution and brine. The organic phase was dried over anhydrous magnesium sulphate, filtered and evaporated under reduced pressure. The residue was purified by flash chromatography on silica gel eluting with 20% ethyl acetate in iso-hexane to yield the sub-titled compound (0.49 g).

¹H NMR (400 MHz, D₆-DMSO) δ 9.60 (s, 1H), 8.09 (d, 1H), 7.92 (d, 1H), 7.80-7.78 (m, 1H), 7.57-7.49 (m, 2H), 7.45-7.41 (m, 2H), 3.74 (t, 2H), 3.58 (t, 2H), 3.32-3.28 (m, 2H), 2.76-2.73 (m, 2H), 2.69-2.63 (m, 4H).

### c) 7-[(1R)-2-Azido-1-hydroxyethyl]-4-(benzyloxy)-1,3-benzothiazol-2(3H)-one

To a solution of 4-(benzyloxy)-7-[(1R)-2-bromo-1-hydroxyethyl]-1,3-benzothiazol-2(3H)-one (prepared by the method outlined in WO 2004/016578, 340 mg) in dimethyl sulfoxide (8 mL) was added sodium azide (231 mg) and sodium iodide (147 mg). The reaction mixture was heated at 65° C. for 5 hours. At the end of this time the mixture was partitioned between ethyl acetate and water, the organic phase was washed with water, dried with anhydrous magnesium sulphate, filtered and concentrated under reduced pressure. The residue was purified by flash chromatography on silica gel eluting with 20% ethyl acetate in toluene to yield the sub-titled compound (195 mg).

¹H NMR (400 MHz, D₆-DMSO) δ 11.89 (s, 1H), 7.54 (d, 2H), 7.38 (t, 2H), 7.33-7.29 (m, 1H), 7.02 (s, 2H), 6.13 (d, 1H), 5.25 (s, 2H), 4.81-4.77 (m, 1H), 3.40-3.27 (m, 2H).

### d) 7-[(1R)-2-Amino-1-hydroxyethyl]-4-(benzyloxy)-1,3-benzothiazol-2(3H)-one hydrochloride

A solution of 7-((1R)-2-azido-1-hydroxyethyl)-4-benzyloxy-3H-benzothiazol-2-one (Example 1c), 195 mg) in a mixture of ethanol (8 mL) and tetrahydrofuran (4 mL) was treated with 110% w palladium on carbon catalyst (20 mg) and the resultant mixture stirred vigorously under 3 atmospheres pressure of hydrogen gas for 20 hours. The catalyst was filtered off and the solvent was removed under reduced pressure. The residue was purified by flash chromatography on silica gel eluting with 1% concentrated aqueous ammonia and 12% methanol in dichloromethane. The resultant product was dissolved in 1,4-dioxane and treated dropwise with a 4 molar solution of hydrogen chloride in 1,4 dioxane (0.5 mL). Evaporation of the solvent under reduced pressure yielded the sub-titled compound (160 mg).

m/e 315 (M−H)⁺

¹H NMR (400 MHz, D₆-DMSO) δ 8.01 (s, 2H), 7.55 (d, 2H), 7.39 (t, 2H), 7.31 (t, 1H), 7.04 (q, 2H), 6.39 (d, 1H), 5.26 (s, 2H), 4.83 (dt, 1H), 2.97-2.83 (m, 2H).

### e) 4-(Benzyloxy)-7-((1R)-1-hydroxy-2-{[3-({2-[2-(1-naphthyl)ethoxy]ethyl}thio)propyl]amino}ethyl)-1,3-benzothiazol-2(3H)-one

A solution of 7-((1R)-2-amino-1-hydroxyethyl)-4-benzyloxy-3H-benzothiazol-2-one hydrochloride (Example 1d), 157 mg) in methanol (6 mL) was treated with 3-[2-(2-naphthalen-1-yl-ethoxy)-ethylsulfanyl]-propionaldehyde (Example 1b), 128 mg) and acetic acid (30 mg). The mixture was stirred at room temperature for 10 minutes and then treated with sodium cyanoborohydride (17 mg). Stirring was continued at room temperature for a further 20 hours and at the end of this time the solvent was removed under reduced pressure and the residue obtained partitioned between dilute aqueous ammonia and ethyl acetate. The organic phase was washed with water, dried with anhydrous magnesium sulphate, filtered and concentrated under reduced pressure to give a crude product. The crude product was purified by flash chromatography on silica gel eluting with 1% concentrated aqueous ammonia and 6% methanol in dichloromethane to yield the sub-titled compound (94 mg).

m/e 589 (M+H⁺, 100%)

### f) 4-Hydroxy-7-((1R)-1-hydroxy-2-{[3-({2-[2-(1-naphthyl)ethoxy]ethyl}thio)propyl]-amino}ethyl)-1,3-benzothiazol-2(3H)-one trifluoroacetate

A solution of 4-(benzyloxy)-7-((1R)-1-hydroxy-2-{[3-({2-[2-(1-naphthyl)ethoxy]ethyl}thio)propyl]amino}ethyl)-1,3-benzothiazol-2(3H)-one (Example 1e), 94 mg) in 98% formic acid (5 mL) was treated with palladium black (10 mg) and the mixture was stirred vigorously at room temperature under nitrogen. Further 10 mg portions of palladium black were added at 30 minute intervals over a 5 hour period. At the end of this time the mixture was filtered and the resultant solution evaporated under reduced pressure. The residue obtained was purified by reverse phase HPLC using a gradient elution of 5% to 75% acetonitrile in 0.2% aqueous trifluoroacetic acid to yield the titled compound (30 mg).

m/e 499 (M+H)⁺

¹H NMR (400 MHz, D₆-DMSO) δ 11.67 (s, 1H), 10.22 (s, 1H), 8.61 (s, 2H), 8.08 (d, 1H), 7.92 (d, 1H), 7.81-7.78 (m, 1H), 7.58-7.40 (m, 4H), 6.93 (d, 1H), 6.77 (d, 1H), 6.45 (d, 1H), 4.88-4.85 (m, 1H), 3.74 (t, 2H), 3.59 (t, 2H), 3.05-2.95 (m, 4H), 2.65 (t, 2H), 1.91-1.81 (m, 2H).

### EXAMPLE 2

### 4-Hydroxy-7-((1R)-1-hydroxy-2-{[3-({2-[2-(1-naphthyl)ethoxy]ethyl}sulfonyl)propyl]-amino}ethyl)-1,3-benzothiazol-2(3H)-one trifluoroacetate

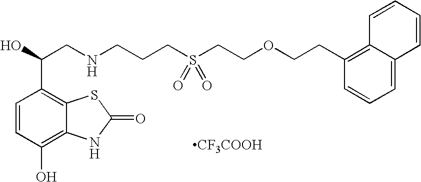

A solution of 4-hydroxy-7-((1R)-1-hydroxy-2-{[3-({2-[2-(1-naphthyl)ethoxy]ethyl}thio)propyl]amino}ethyl)-1,3-benzothiazol-2(3H)-one trifluoroacetate (Example 1, 83 mg) in ethanol (5 mL) was treated with 3-chloroperoxybenzoic acid (62 mg of 75% grade reagent) and the solution was stirred for 2 hours at room temperature. The solvent was evaporated off under a stream of nitrogen gas and the residue was purified by flash chromatography on silica gel eluting with 1% concentrated aqueous ammonia and 10% methanol in dichloromethane. The resultant product was further purified by reverse phase HPLC using a gradient elution of 5% to 75% acetonitrile in 0.2% aqueous trifluoroacetic acid to yield the titled compound (30 mg).

m/e 531 (M+H)⁺

¹H NMR (400 MHz, D₆-DMSO) δ 11.68 (s, 1H), 10.22 (s, 1H), 8.68 (s, 2H), 8.08 (d, 1H), 7.91 (d, 1H), 7.79 (d, 1H), 7.57-7.49 (m, 2H), 7.46-7.40 (m, 2H), 6.94 (d, 1H), 6.78 (d, 1H), 6.47 (d, 1H), 4.87-4.83 (m, 1H), 3.81-3.76 (m, 4H), 3.38 (t, 2H), 3.11-3.00 (m, 6H), 2.05-1.99 (m, 2H).

### EXAMPLE 3

### 4-Hydroxy-7-[(1R)-1-hydroxy-2-({3-[2-(2-phenylethoxy)ethoxy]propyl}amino)ethyl]-1,3-benzothiazol-2(3H)-one trifluoroacetate

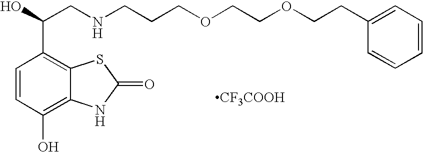

### a) 4-(Benzyloxy)-7-[(1R)-1-hydroxy-2-({3-[2-(2-phenylethoxy)ethoxy]propyl}amino)ethyl]-1,3-benzothiazol-2(3H)-one

The sub-titled compound was prepared from 7-[(1R)-2-amino-1-hydroxyethyl]-4-(benzyloxy)-1,3-benzothiazol-2(3H)-one hydrochloride (Example 1d), 250 mg) and 3-(2-(2-phenylethoxy)ethoxy)-propanal (prepared as described in WO 93/24473, 157 mg) using the method of Example 1e). The crude product was purified by flash chromatography on silica gel eluting with 1% concentrated aqueous ammonia and 8% methanol in dichloromethane to yield the sub-titled compound (160 mg).

m/e 523 (M+H)⁺

### b) 4-hydroxy-7-[(1R)-1-hydroxy-2-({3-[2-(2-phenylethoxy)ethoxy]propyl}-amino)ethyl]-1,3-benzothiazol-2(3H)-one trifluoroacetate

The titled compound was prepared from 4-(benzyloxy)-7-[(1R)-1-hydroxy-2-({3-[2-(2-phenylethoxy)ethoxy]propyl}amino)ethyl]-1,3-benzothiazol-2(3H)-one (Example 3a), 160 mg) using the method of Example 1f). The crude product was purified by reverse phase HPLC and a gradient elution of 5% to 75% acetonitrile in 0.2% aqueous trifluoroacetic acid to yield the titled compound (40 mg).

m/e 433 (M+H)⁺

¹H NMR (400 MHz, D₆-DMSO) δ 11.67 (s, 1H), 10.26 (s, 1H), 8.67 (s, 1H), 8.56 (s, 1H), 7.30-7.15 (m, 5H), 6.92 (d, 1H), 6.77 (d, 1H), 6.46 (s, 1H), 4.89-4.86 (m, 1H), 3.60 (t, 2H), 3.43 (t, 2H), 3.03-3.01 (m, 4H), 2.80 (t, 2H), 1.93-1.80 (m, 2H).

### EXAMPLE 4

### 4-Hydroxy-7-((1R)-1-hydroxy-2-{[2-({2-[2-(1-naphthyl)ethoxy]ethyl}thio)ethyl]-amino}ethyl)-1,3-benzothiazol-2(3H)-one hydrochloride

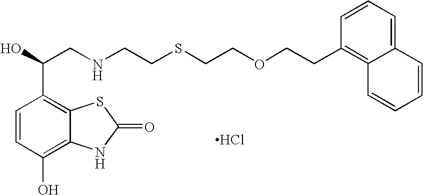

### a) 2-[2-(2-Naphthalen-1-yl-ethoxy)-ethylsulfanyl]-ethanol

1-(2-Vinyloxy-ethyl)-naphthalene (1.95 g) and 2-mercaptoethanol (0.78 g, 0.7 mL) were mixed together and 2,2′-azobisisobutyronitrile (40 mg) was added. The mixture was heated to 50° C. for 2 hours, cooled and diluted with dichloromethane (5 mL). The solution was purified by chromatography on silica gel eluting with ethyl acetate/iso-hexane (1/9 to 1/1) to give the sub-titled compound (2.04 g) as an oil.

¹H NMR (400 MHz, CDCl₃) δ 8.06 (d, 1H), 7.85 (d, 1H), 7.74 (d, 1H), 7.54-7.46 (m, 2H), 7.43-7.37 (m, 2H), 3.82 (t, 2H), 3.71 (q, 4H), 3.65 (t, 3H), 3.39 (t, 2H), 2.73 (t, 4H).

### b) 7-[(1R)-2-Amino-1-hydroxyethyl]-4-hydroxy-1,3-benzothiazol-2(3H)-one hydrochloride

A solution of 7-[(1R)-2-amino-1-hydroxyethyl]-4-(benzyloxy)-1,3-benzothiazol-2(3H)-one hydrochloride (Example 1d), 1.8 g) in methanol (60 mL) and concentrated hydrochloric acid (4 mL) was stirred vigorously in the presence of 10% w palladium on carbon catalyst (0.36 g) and under 4 atmospheres pressure of hydrogen gas for 2 hours. Further 10% w palladium on carbon catalyst (0.24 g) was added and stirring continued under hydrogen for 1 hour. The catalyst was filtered off and the solvent evaporated under reduced pressure to yield the sub-titled compound (1.3 g).

m/e 227 (M+H)⁺

¹H NMR (400 MHz, D₆-DMSO) δ 11.70 (s, 1H), 10.21 (s, 1H), 8.04 (s, 3H), 6.92 (d, 1H), 6.79 (d, 1H), 6.32 (d, 1H), 4.81-4.79 (m, 1H), 2.90-2.81 (m, 2H).

### c) 4-Hydroxy-7-((1R)-1-hydroxy-2-{[2-({2-[2-(1-naphthyl)ethoxy]ethyl}thio)ethyl]-amino}ethyl)-1,3-benzothiazol-2(3H)-one hydrochloride

To a solution of 2-[2-(2-naphthalen-1-yl-ethoxy)-ethylsulfanyl]-ethanol (Example 4a), 0.21 g) in dichloromethane (10 mL) was added pyridinium chlorochromate (0.484 g) and the mixture was stirred for 2 hours. The reaction mixture was purified by chromatography on silica gel (10 mm×20 mm, the silica column being flushed with ethyl acetate/iso-hexane 1:1). The residue, after evaporation under reduced pressure was dissolved in methanol (5 mL) and 7-[(1R)-2-amino-1-hydroxyethyl]-4-hydroxy-1,3-benzothiazol-2(3H)-one hydrochloride (Example 4b), 0.113 g) and acetic acid (0.05 mL) were added and the mixture was stirred for 1 hour. Sodium cyanoborohydride (0.031 g) was added and the reaction mixture was stirred overnight. Concentrated aqueous ammonia (0.1 mL) was added and the mixture concentrated onto silica gel and the residue purified by chromatography on silica gel eluting with 1-10% 0.7N ammonia in methanol in dichloromethane to afford the product which was re-purified by reverse phase HPLC using 5-95% 0.2M trifluoroacetic acid in acetonitrile. The compound-containing fractions were concentrated and the residue was dissolved in 4N hydrogen chloride in 1,4-dioxane (5 mL) and concentrated. The residue was triturated with ether and the ether decanted off to afford the titled compound (0.028 g) as a white solid.

m/e 485 (M+H)⁺

¹H NMR (400 MHz, D₆-DMSO) δ 11.69 (s, 1H), 10.20 (s, 1H), 8.90 (s, 1H), 8.64 (s, 1H), 8.08 (d, 1H), 7.91 (d, 1H), 7.79 (dd, 1H), 7.57-7.48 (m, 2H), 7.43 (q, 2H), 6.91 (d, 1H), 6.78 (d, 1H), 6.44 (d, 1H), 4.93-4.89 (m, 1H), 3.75 (t, 2H), 3.60 (t, 2H), 3.30 (t, 2H), 3.16-3.09 (m, 2H), 3.06-2.99 (m, 2H), 2.85-2.76 (m, 2H), 2.71 (t, 2H).

### EXAMPLE 5

### 7-((1R)-2-{[3-({3-[2-(4-Bromophenyl)ethoxy]propyl}thio)propyl]amino}-1-hydroxyethyl)-4-hydroxy-1,3-benzothiazol-2(3-H)-one hydrochloride

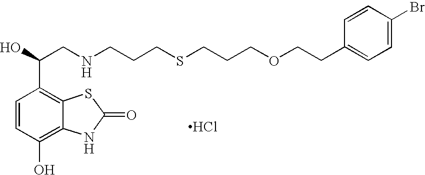

### a) Toluene-4-sulfonic acid 3-allylsulfanyl-propyl ester

A solution of 3-(2-propenylthio)-1-propanol (3.7 g) in dichloromethane (25 mL) was treated with triethylamine (3.12 g), 4-(dimethylamino)pyridine (0.2 g) and tosyl chloride (5.87 g) and the mixture was stirred at room temperature for 18 hours. The reaction mixture was diluted with chloroform, washed with water and the organic layer was evaporated under reduced pressure. The crude product was purified by flash chromatography on silica gel eluting with 40% diethyl ether in isohexane to yield the sub-titled compound (5.22 g).

¹H NMR (400 MHz, CDCl₃) δ 7.79 (d, 2H), 7.35 (d, 2H), 5.73-5.69 (m, 1H), 5.08-5.04 (m, 2H), 4.13 (t, 2H), 3.06 (d, 2H), 2.47-2.43 (m, 5H), 1.91-1.87 (m, 2H).

### b) 1-[2-(3-Allylsulfanyl-propoxy)-ethyl]-4-bromo-benzene

A solution of 4-bromophenethylalcohol (4.12 g) in tetrahydrofuran (10 mL) was added dropwise to a stirred suspension of sodium hydride (0.8 g of 60% grade sodium hydride) in tetrahydrofuran (20 mL) at 0° C. The mixture was stirred at room temperature for 1 hour and then cooled to 0° C., to which a solution of toluene-4-sulfonic acid 3-allylsulfanylpropyl ester (Example 5a), 5.2 g) in terahydrofuran (10 mL) was added dropwise. The reaction mixture was heated to reflux for 3 hours and then stirred at room temperature for 18 hours. The reaction mixture was quenched by the addition of excess dilute aqueous hydrochloric acid. The mixture was then basified by addition of saturated aqueous sodium bicarbonate and was extracted with ethyl acetate. The organic layer was washed with aqueous brine, dried over magnesium sulphate and the solvent was removed under reduced pressure. The crude product was purified by flash chromatography on silica gel eluting with 20% ethyl acetate in iso-hexane to yield the sub-titled compound (2.52 g).

¹H NMR (400 MHz, CDCl₃) δ 7.46-7.05 (m, 4H), 5.85-5.70 (m, 1H), 5.14-5.02 (m, 2H), 3.65-3.56 (m, 2H), 3.54-3.45 (m, 2H), 3.15-3.07 (m, 2H), 2.87-2.77 (m, 2H), 2.53-2.46 (m, 2H), 1.88-1.73 (m, 2H).

### c) 3-{3-[2-(4-Bromophenyl)-ethoxy]-propylsulfanyl}-propan-1-ol

To a solution of 1-[2-(3-allylsulfanyl-propoxy)-ethyl]-4-bromo-benzene (Example 5b), 2.2

g) in tetrahydrofuran (40 mL) was added, dropwise, a 0.5 molar solution of 9-borabicyclo[3.3.1]nonane in tetrahydrofuran (28 mL). The mixture was heated at reflux for 1 hour. The reaction mixture was then cooled to room temperature and treated with a 3 molar solution of aqueous sodium hydroxide (2.56 mL) followed by a 35% solution of aqueous hydrogen peroxide (2.28 mL). The mixture was stirred at room temperature for 1 hour and then treated with solid sodium chloride. The solvent was decanted off, dried and evaporated under reduced pressure. The crude product was purified by flash chromatography on silica gel eluting with 33% ethyl acetate in iso-hexane to yield the sub-titled compound (1.48 g).

¹H NMR (400 MHz, D₆-DMSO) δ 7.45 (d, 2H), 7.20 (d, 2H), 4.45 (t, 1H), 3.55 (t, 2H), 3.46-3.41 (m, 4H), 2.76 (t, 2H), 2.49-2.42 (m, 4H), 1.72-1.58 (m, 4H).

### d) 3-{3-[2-(4-Bromophenyl)-ethoxy]-propylsulfanyl}-propionaldehyde

The sub-titled compound was prepared from 3-{3-[2-(4-bromo-phenyl)-ethoxy]-propylsulfanyl}-propan-1-ol (Example 5c), 620 mg) using the method of Example 1b). The crude product was purified by flash chromatography on silica gel eluting with 20% ethyl acetate in iso-hexane to yield the sub-titled compound (200 mg).

¹H NMR (400 MHz, DMSO) δ 9.63 (s, 1H), 7.46 (d, 2H), 7.19 (d, 2H), 3.55 (t, 2H), 3.43 (t, 2H), 2.77 (t, 2H), 2.73-2.66 (m, 4H), 1.73-1.67 (m, 2H).

### e) 7-((1R)-2-{[3-({3-[2-(4-Bromophenyl)ethoxy]propyl}thio)propyl]amino}-1-hydroxyethyl)-4-hydroxy-1,3-benzothiazol-2(3H)-one hydrochloride

To a solution of 7-[(1R)-2-amino-1-hydroxyethyl]-4-hydroxy-1,3-benzothiazol-2(3H)-one hydrochloride (Example 4b, 100 mg) in methanol (7 mL) was added 3-{3-[2-(4-bromophenyl)-ethoxy]-propylsulfanyl}-propionaldehyde (Example 5d), 126 mg) and acetic acid (20 mg). The mixture was stirred at room temperature for 40 minutes and then treated with sodium cyanoborohydride (14 mg). The reaction mixture was stirred at room temperature for 18 hours. At the end of this time the solvent was evaporated under reduced pressure and the residue was partitioned between ethyl acetate (50 mL) and saturated aqueous brine (50 mL) containing concentrated aqueous ammonia (1 mL). The organic layer was separated, dried and evaporated under reduced pressure. The crude product was purified by flash chromatography on silica gel eluting with 1% concentrated aqueous ammonia and 9% methanol in dichloromethane. The product was dissolved in methanol and treated with excess hydrogen chloride in dioxane. The solvent was then evaporated under reduced pressure to yield the titled compound (60 mg).

m/e 541 (M+H⁺, 100%)

¹H NMR (400 MHz, D₆-DMSO) δ 11.69 (s, 1H), 10.20 (s, 1H), 8.90 (s, 1H), 8.69 (s, 1H), 7.47 (d, 2H), 7.19 (d, 2H), 6.93 (d, 1H), 6.78 (d, 1H), 6.43 (d, 1H), 4.93-4.90 (m, 1H), 3.56 (t, 2H), 3.43 (t, 2H), 3.02-2.97 (m, 4H), 2.77 (t, 2H), 1.92-1.84 (m, 2H), 1.75-1.68 (m, 2H).

### EXAMPLE 6

### 4-Hydroxy-7-{(1R)-1-hydroxy-2-[(3-{[3-(2-phenylethoxy)propyl]thio}propyl)amino]ethyl}-1,3-benzothiazol-2(3H)-one hydrochloride

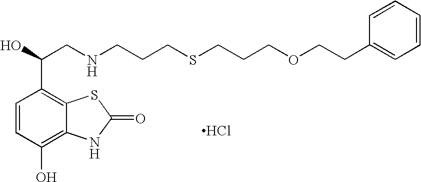

### a) 3-(3-Phenethyloxy-propylsulfanyl)-propan-1-ol

A mixture of (2-allyloxy-ethyl)-benzene (885 mg), 3-mercapto-1-propanol (503 mg) and 2,2′-azobisisobutyronitrile (20 mg) was stirred under nitrogen at 60° C. for 1 hour. The mixture was cooled to room temperature and purified by flash chromatography on silica gel eluting with 33% ethyl acetate in isohexane to yield the sub-titled compound (810 mg).

¹H NMR (400 MHz, D₆-DMSO) δ 7.29-7.16 (m, 5H), 4.45 (t, 1H), 3.56 (t, 2H), 3.46-3.42 (m, 4H), 2.79 (t, 2H), 2.48-2.44 (m, 4H), 1.74-1.59 (m, 4H).

### b) 3-(3-Phenethyloxy-propylsulfanyl)-propionaldehyde

The sub-titled compound was prepared from 3-(3-phenethyloxy-propylsulfanyl)-propan-1-ol (800 mg) using the method of Example 1b). The crude product was purified by flash chromatography on silica gel eluting with 20% ethyl acetate in iso-hexane to yield the sub-titled compound (170 mg).

¹H NMR (400 MHz, D₆-DMSO) δ 9.63 (s, 1H), 7.30-7.16 (m, 5H), 3.57 (t, 2H), 3.44 (t, 2H), 2.79 (t, 2H), 2.74-2.67 (m, 4H), 1.75-1.66 (m, 2H).

### c) 4-Hydroxy-7-{(1R)-1-hydroxy-2-[(3-{[3-(2-phenylethoxy)propyl]thio}propyl)amino]ethyl}-1,3-benzothiazol-2(3H)-one hydrochloride

The titled compound was prepared from 7-[(1R)-2-amino-1-hydroxyethyl]-4-hydroxy-1,3-benzothiazol-2(3H)-one hydrochloride (Example 4b), 100 mg) and 3-(3-phenethyloxypropylsulfanyl)-propionaldehyde (Example 6b), 96 mg) using the method of Example 5e). The crude product was purified by flash chromatography on silica gel eluting with 1% concentrated aqueous ammonia and 10% methanol in dichloromethane. The product was dissolved in methanol and treated with excess hydrogen chloride in dioxane. The solvent was then evaporated under reduced pressure to yield the titled compound (40 mg).

m/e 463 (M+H⁺, 100%)

¹H NMR (400 MHz, D₆-DMSO) δ 11.69 (s, 1H), 10.21 (s, 1H), 8.93 (s, 1H), 8.70 (s, 1H), 7.29-7.16 (m, 5H), 6.93 (d, 1H), 6.78 (d, 1H), 6.43 (d, 1H), 4.94-4.92 (m, 1H), 3.57 (t, 2H), 3.45 (t, 2H), 3.01-2.96 (m, 4H), 2.79 (t, 2H), 1.92-1.84 (m, 2H), 1.76-1.69 (m, 2H).

### EXAMPLE 7

### 4-Hydroxy-7-{(1R)-1-hydroxy-2-[(3-{2-[2-(1-naphthyl)ethoxy]ethoxy}propyl)amino]ethyl}-1,3-benzothiazol-2(3H)-one

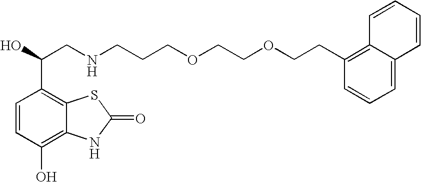

### a) (2-Naphthalen-1-yl-ethoxy)-acetic acid

To a solution of 1-naphthaleneethanol (2.85 g) in N,N-dimethylformamide (40 mL) was added, portionwise, 60% grade sodium hydride (1.33 g) and the resultant mixture was stirred at 60° C. for 10 minutes before being cooled to 40° C. Chloroacetic acid (1.56 g) was added in portions and the reaction mixture was then heated at 60° C. for 30 minutes. After cooling to room temperature the reaction mixture was quenched by the careful addition of water and then partitioned between water and diethylether. The aqueous layer was acidified by addition of 2 molar aqueous hydrochloric acid and extracted with ethyl acetate. The ethyl acetate layer was washed with brine, dried and the solvent was removed under reduced pressure to yield the sub-titled compound (2.9 g).

m/e 229 (M−H⁺, 100%)

### b) 2-(2-Naphthalen-1-yl-ethoxy)-ethanol

A solution of (2-naphthalen-1-yl-ethoxy)-acetic acid (Example 7a), 2.9 g) in tetrahydrofuran (60 mL) was treated dropwise with a 2 molar solution of borane-methyl sulphide complex in tetrahydrofuran (12.6 mL) and the mixture was stirred at room temperature for 2 hours. Methanol (20 mL) was added cautiously and stirring was continued for 30 minutes. At the end of this time the solvents were removed under reduced pressure and the residue was partitioned between diethylether and saturated aqueous potassium carbonate solution. The organic layer was dried and then evaporated under reduced pressure to yield the crude product which was purified by flash chromatography on silica gel eluting with ethyl acetate in iso-hexane (1/3) to yield the sub-titled compound (2.03 g).

¹H NMR (400 MHz, D₆-DMSO) δ 8.06 (d, 1H), 7.89 (d, 1H), 7.80-7.74 (m, 1H), 7.55-7.44 (m, 2H), 7.43-7.38 (m, 2H), 4.55 (t, 1H), 3.70 (t, 2H), 3.50-3.42 (m, 4H), 3.28 (t, 2H).

### c) 3-[2-(2-Naphthalen-1-yl-ethoxy)-ethoxy]-propionitrile

A mixture of 2-(2-naphthalen-1-yl-ethoxy)-ethanol (Example 7b), 2.03 g), 3-bromopropanenitrile (1.09 mL), sodium hydroxide (10 g) and tetrabutylammonium chloride (0.1 g) in dichloromethane (20 mL) and water (20 mL) was stirred vigorously for 18 hours. The reaction mixture was partitioned between water and dichloromethane, the organic layer was washed with dilute aqueous hydrochloric acid and then with water before being dried and evaporated under reduced pressure to yield the crude product. Purification by flash chromatography on silica gel eluting with 33% ethyl acetate in iso-hexane gave the sub-titled compound (1.5 g).

¹H NMR (400 MHz, D₆-DMSO) δ 8.06 (d, 1H), 7.88 (d, 1H), 7.77-7.74 (m, 1H), 7.52-7.46 (m, 2H), 7.42-7.38 (m, 2H), 3.71 (t, 2H), 3.55-3.52 (m, 6H), 2.67 (t, 2H).

### d) 3-[2-(2-Naphthalen-1-yl-ethoxy)-ethoxy]-propionic acid

A mixture of 3-[2-(2-naphthalen-1-yl-ethoxy)-ethoxy]-propionitrile (Example 7c), 1.5 g) in concentrated hydrochloric acid (20 mL) and water (10 mL) was refluxed with vigorous stirring for 5 hours and then cooled to room temperature. The reaction mixture was partitioned between diethyl ether and water and the organic layer extracted with 1 molar aqueous sodium hydroxide solution. The aqueous sodium hydroxide layer was acidified by addition of excess concentrated hydrochloric acid and the mixture was extracted with ethyl acetate, washed with brine, dried under anhydrous magnesium sulphate and evaporated under reduced pressure to yield the sub-titled compound (0.87 g).

m/e 287 (M−H⁺, 100%)

### e) 3-[2-(2-Naphthalen-1-yl-ethoxy)-ethoxy]-propan-1-ol

The sub-titled compound was prepared from 3-[2-(2-naphthalen-1-yl-ethoxy)-ethoxy]-propionic acid (Example 7d), 0.87 g) using the method of Example 7b) to yield 0.73 g of the sub-titled compound.

m/e 275 (M+H⁺, 100%)

### f) 3-[2-(2-Naphthalen-1-yl-ethoxy)-ethoxy]-propionaldehyde

The sub-titled compound was prepared from 3-[2-(2-naphthalen-1-yl-ethoxy)-ethoxy]-propan-1-ol (Example 7e), 0.36 g) using the method of Example 1b). The crude product was purified by flash chromatography on silica gel eluting with 33% ethyl acetate in isohexane to yield the sub-titled compound (226 mg).

¹H NMR (400 MHz, D₆-DMSO) δ 9.62 (t, 1H), 8.08 (d, 1H), 7.93-7.90 (m, 1H), 7.82-7.76 (m, 1H), 7.58-7.46 (m, 2H), 7.44-7.39 (m, 2H), 3.74-3.67 (m, 4H), 3.55-3.48 (m, 4H), 2.60-2.55 (m, 2H).

### g) 4-Hydroxy-7-{(1R)-1-hydroxy-2-[(3-{2-[2-(1-naphthyl)ethoxy]ethoxy}propyl)amino]ethyl}-1,3-benzothiazol-2(3H)-one

To a solution of 7-[(1R)-2-amino-1-hydroxyethyl]-4-hydroxy-1,3-benzothiazol-2(3H)-one hydrochloride (Example 4b), 100 mg) in methanol (7 mL) was added 3-[2-(2-naphthalen-1-yl-ethoxy)-ethoxy]-propionaldehyde (Example 7f), 104 mg) and acetic acid (20 mg). The mixture was stirred at room temperature for 40 minutes and then treated with sodium cyanoborohydride (14 mg). The reaction mixture was stirred at room temperature for 18 hours and at the end of this time the solvent was evaporated under reduced pressure and the residue partitioned between ethyl acetate (50 mL) and brine (50 mL) containing concentrated aqueous ammonia (1 mL). The organic layer was separated, dried and evaporated under reduced pressure. The crude product was purified by flash chromatography on silica gel eluting with 1% concentrated aqueous ammonia and 10% methanol in dichloromethane to yield the titled compound (40 mg).

m/e 483 (M+H⁺, 100%)

¹H NMR (400 MHz, D₆-DMSO) δ 8.08 (d, 1H), 7.90 (d, 1H), 7.78-7.75 (m, 1H), 7.56-7.45 (m, 2H), 7.42-7.39 (m, 2H), 6.85 (d, 1H), 6.69 (d, 1H), 4.57 (q, 1H), 3.73 (t, 2H), 3.54-3.51 (m, 2H), 3.47-3.43 (m, 2H), 3.38 (t, 2H), 3.29 (t, 2H), 2.68-2.54 (m, 2H), 1.63-1.56 (m, 2H).

### EXAMPLE 8

### 4-Hydroxy-7-{(1R)-1-hydroxy-2-[(2-{[3-(2-phenylethoxy)propyl]thio}-ethyl)amino]ethyl}-1,3-benzothiazol-2(3H)-one hydrochloride

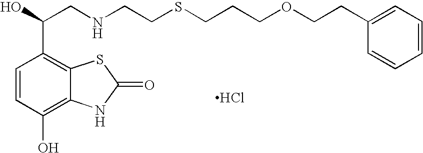

### a) 2-(3-Phenethyloxy-propylsulfanyl)-ethanol

The sub-titled compound was prepared from (2-allyloxy-ethyl)-benzene (647 mg) and 2-mercaptoethanol (312 mg) according to the method of Example 1a). The crude product was purified by flash chromatography on silica gel eluting with 33% ethyl acetate in iso-hexane to yield the sub-titled compound (652 mg).

¹H NMR (400 MHz, D₆-DMSO) δ 7.25-7.12 (m, 5H), 4.70 (t, 1H), 3.52 (t, 2H), 3.45 (q, 2H), 3.39 (t, 2H), 2.75 (t, 2H), 1.70-1.63 (m, 2H).

### b) (3-Phenethyloxy-propylsulfanyl)-acetaldehyde

The sub-titled compound was prepared from 2-(3-phenethyloxy-propylsulfanyl)-ethanol (Example 8a), 0.65 g) according to the method of Example 1b). The crude product was purified by flash chromatography on silica gel eluting with 20% ethyl acetate in iso-hexane to yield the sub-titled compound (95 mg).

¹H NMR (400 MHz, D₆-DMSO) δ 9.40 (t, 1H), 7.30-7.16 (m, 5H), 3.56 (t, 2H), 3.43 (t, 2H), 2.79 (t, 2H), 2.43 (t, 2H), 1.74-1.66 (m, 2H).

### c) 4-Hydroxy-7-{(1R)-1-hydroxy-2-[(2-{[3-(2-phenylethoxy)propyl]-thio}ethyl)amino]ethyl}-1,3-benzothiazol-2(3H)-one hydrochloride

The titled compound was prepared from 7-[(1R)-2-amino-1-hydroxyethyl]-4-hydroxy-1,3-benzothiazol-2(3H)-one hydrochloride (Example 4b), 100 mg) and (3-phenethyloxy-propylsulfanyl)-acetaldehyde (Example 8b), 91 mg) using the method of Example 5e). The crude product was purified by flash chromatography on silica gel eluting with 1% concentrated aqueous ammonia and 10% methanol in dichloromethane. The product was dissolved in methanol and treated with excess hydrogen chloride in 1,4-dioxane. The solvent was then evaporated under reduced pressure to yield the titled compound (40 mg).

m/e 449 (M+H⁺, 100%)

¹H NMR (400 MHz, D₆-DMSO) δ 11.70 (s, 1H), 10.23 (s, 1H), 9.12 (s, 1H), 8.73 (s, 1H), 7.29-7.16 (m, 5H), 6.92 (d, 1H), 6.79 (d, 1H), 6.44 (s, 1H), 4.96-4.93 (m, 1H), 3.57 (t, 2H), 3.45 (t, 2H), 3.12-3.05 (m, 4H), 2.85-2.72 (m, 4H), 2.53 (t, 2H), 1.77-1.71 (m, 2H).

### EXAMPLE 9

### 4-Hydroxy-7-{(1R)-1-hydroxy-2-[(3-{[2-(2-phenylethoxy)ethyl]thio}-propyl)amino]ethyl}-1,3-benzothiazol-2(3H)-one hydrochloride

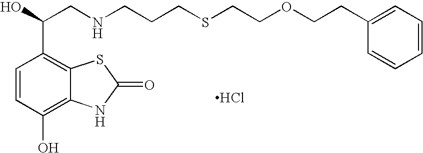

### a) 3-(2-Phenethyloxy-ethylsulfanyl)-propan-1-ol

The sub-titled compound was prepared from 3-[2-(2-phenylethoxy)ethylthio]propanoic acid (1.11 g) according to the method of Example 7b). The crude product was purified by flash chromatography on silica gel, eluting with 50% ethyl acetate in dichloromethane to yield the sub-titled compound (0.7 g).

¹H NMR (400 MHz, CDCl₃) δ 7.32-7.19 (m, 5H), 3.75-3.68 (m, 2H), 3.66-3.61 (m, 4H), 2.92-2.86 (m, 2H), 2.73-2.62 (m, 4H), 1.89-1.80 (m, 2H).

### b) 3-(2-Phenethyloxy-ethylsulfanyl)-propionaldehyde

To a solution of 3-(2-phenethyloxy-ethylsulfanyl)-propan-1-ol (Example 9a), 330 mg) and triethylamine (417 mg) in dichloromethane (3 mL) cooled to −10° C. was added in one portion a solution of sulphur trioxide-pyridine complex (652 mg) in dimethylsulphoxide (3 mL). The cooling bath was removed and the mixture was stirred vigorously for 10 minutes. The mixture was then partitioned between ethyl acetate and brine, the organic layer was washed with a 10% aqueous solution of citric acid and then with aqueous brine before being dried and evaporated under reduced pressure. The crude product was purified by flash chromatography on silica gel eluting with 20% ethyl acetate in isohexane to yield the sub-titled compound (165 mg).

¹H NMR (400 MHz, D₆-DMSO) δ 9.61 (s, 1H), 7.31-7.17 (m, 5H), 3.64-3.53 (m, 4H), 2.83-2.63 (m, 8H).

### c) 4-Hydroxy-7-{(1R)-1-hydroxy-2-[(3-{[2-(2-phenylethoxy)ethyl]thio}-propyl)amino]ethyl}-1,3-benzothiazol-2(3H)-one hydrochloride

The titled compound was prepared from 7-[(1R)-2-amino-1-hydroxyethyl]-4-hydroxy-1,3-benzothiazol-2(3H)-one hydrochloride (Example 4b), 100 mg) and 3-(2-phenethyloxyethylsulfanyl)-propionaldehyde (Example 9b), 91 mg) using the method of Example 5e). The crude product was purified by flash chromatography on silica gel eluting with 1% concentrated aqueous ammonia and 10% methanol in dichloromethane. The product was dissolved in methanol and treated with excess hydrogen chloride in dioxane. The solvent was the evaporated under reduced pressure to yield the titled compound (50 mg).

m/e 449 (M+H⁺, 100%)

¹H NMR (400 MHz, D₆-DMSO) δ 11.69 (s, 1H), 10.21 (s, 1H), 8.97 (s, 1H), 8.71 (s, 1H), 7.30-7.16 (m, 5H), 6.93 (d, 1H), 6.79 (d, 1H), 6.43 (s, 1H), 4.95-4.93 (m, 1H), 3.61 (t, 2H), 3.55 (t, 2H), 3.02-2.95 (m, 4H), 2.80 (t, 2H), 2.65 (t, 2H), 2.59 (t, 2H), 1.95-1.83 (m, 2H).

### EXAMPLE 10

### tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}(2-phenylethyl)carbamate

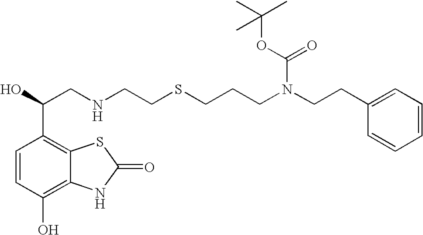

### a) [3-(2-Hydroxy-ethylsulfanyl)-propyl]-phenethyl-carbamic acid tert-butyl ester

A mixture of allyl-phenethyl-carbamic acid tert-butyl ester (520 mg), 2-mercaptoethanol (156 mg) and 2,2′-azobis(2-methylpropionitrile) (20 mg) was heated at 60° C. under nitrogen for 45 minutes. The crude product was purified by flash chromatography on silica gel eluting with 33% ethyl acetate in iso-hexane to yield the sub-titled compound (420 mg).

¹H NMR (400 MHz, D₆-DMSO) δ 7.31-7.27 (m, 2H), 7.21-7.18 (m, 3H), 4.75 (t, 1H), 3.52 (q, 2H), 3.16 (t, 2H), 2.75 (t, 2H), 2.55 (t, 2H), 2.45 (t, 2H), 1.74-1.64 (m, 2H), 1.35 (s, 9H).

### b) [3-(2-Oxo-ethylsulfanyl)-propyl]-phenethyl-carbamic acid tert-butyl ester

The sub-titled compound was prepared from [3-(2-hydroxy-ethylsulfanyl)-propyl]-phenethyl-carbamic acid tert-butyl ester (Example 10a), 406 mg) according to the method of Example 9b). The crude product was purified by flash chromatography on silica gel eluting with 20% ethyl acetate in iso-hexane to yield the sub-titled compound (274 mg).

m/e 336.7 (M−H⁺, 100%)

### c) tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}(2-phenylethyl)carbamate

The titled compound was prepared from 7-[(1R)-2-amino-1-hydroxyethyl]-4-hydroxy-1,3-benzothiazol-2(3H)-one hydrochloride (Example 4b), 163 mg) and [3-(2-oxo-ethylsulfanyl)-propyl]-phenethyl-carbamic acid tert-butyl ester (Example 10b), 209 mg) according to the method of Example 5e). The crude product was purified by flash chromatography on silica gel, eluting with 1% concentrated aqueous ammonia and 10% methanol in dichloromethane to yield the titled compound (157 mg).

m/e 548 (M+H⁺, 100%)

¹H NMR (400 MHz, D₆-DMSO) δ 11.20-10.80 (m, 1H), 9.95 (s, 1H), 7.29-7.26 (m, 2H), 7.20-7.17 (m, 3H), 6.85 (d, 1H), 6.68 (d, 1H), 5.60-5.40 (m, 1H), 4.55 (q, 1H), 3.16-3.14 (m, 2H), 2.74 (t, 2H), 2.70-2.54 (m, 6H), 2.41 (t, 2H), 1.69-1.62 (m, 2H), 1.33 (s, 9H).

### EXAMPLE 11

### 4-Hydroxy-7-((1R)-1-hydroxy-2-{[2-({3-[(2-phenylethyl)amino]propyl}thio)ethyl]amino}ethyl)-1,3-benzothiazol-2(3H)-one hydrochloride

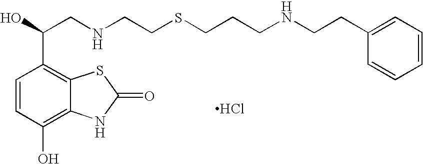

A solution of tert-butyl {3-[(2-{[(2R)-2-hydroxy-2-(4-hydroxy-2-oxo-2,3-dihydro-1,3-benzothiazol-7-yl)ethyl]amino}ethyl)thio]propyl}(2-phenylethyl)carbamate prepared as described in Example 10 (145 mg) in methanol (7 mL) was treated with a 4 molar solution of hydrogen chloride in 1,4-dioxane (2 mL) and allowed to stand for 18 hours at room temperature. The solvents were evaporated under reduced pressure and the residue was purified by reverse phase HPLC using a gradient elution of 5% to 75% acetonitrile in 0.2% aqueous ammonia. The product was further purified by flash chromatography on silica gel, eluting with 1% concentrated aqueous ammonia and 10% methanol in dichloromethane. The product was dissolved in methanol and treated with excess hydrogen chloride in 1,4-dioxane. The solvent was then evaporated under reduced pressure to yield the titled compound (44 mg).

m/e 448 (M+H⁺, 100%)

¹H NMR (400 MHz, D₆-DMSO) δ 11.69 (s, 1H), 10.22 (s, 1H), 9.25 (s, 1H), 9.16 (s, 2H), 8.80 (s, 1H), 7.35-7.31 (m, 2H), 7.27-7.23 (m, 3H), 6.92 (d, 1H), 6.78 (d, 1H), 6.44 (d, 1H), 4.99-4.97 (m, 1H), 3.15-2.96 (m, 10H), 2.85-2.83 (m, 2H), 2.67 (t, 2H), 1.97-1.92 (m, 2H).

### EXAMPLE 12

### 4-Hydroxy-7-((1R)-1-hydroxy-2-{[2-({3-[methyl(2-phenylethyl)amino]propyl}-thio)ethyl]amino}ethyl)-1,3-benzothiazol-2(3H)-one trifluoroacetate

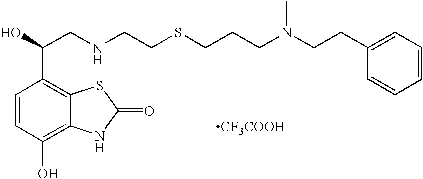

### a) [3-(2-Hydroxy-ethylsulfanyl)-propyl]-methyl-carbamic acid tert-butyl ester

The sub-titled compound was prepared according to the method of Example 1a) using allyl-methyl-carbamic acid tert-butyl ester (5.97 g) and 2-mercaptoethanol (2.86 g). The crude product was purified by flash chromatography on silica gel eluting with 50% ethyl acetate in iso-hexane to yield the sub-titled compound (5.1 g).

¹H NMR (400 MHz, D₆-DMSO) δ 4.77 (t, 1H), 3.57-3.50 (m, 2H), 3.23 (t, 2H), 2.78 (s, 3H), 2.56 (t, 2H), 2.52-2.46 (m, 2H), 1.76-1.66 (m, 2H), 1.41 (s, 9H).

### b) 2-(3-Methylamino-propylsulfanyl)-ethanol hydrochloride

A solution of [3-(2-hydroxy-ethylsulfanyl)-propyl]-methyl-carbamic acid tert-butyl ester (Example 12a), 5.1 g) in methanol (40 mL) was treated with a 4 molar solution of hydrogen chloride in 1,4-dioxane (10 mL) and the mixture was allowed to stand at room temperature for 5 hours. The solvents were evaporated under reduced pressure and the residue was triturated with diethyl ether to yield the sub-titled compound (3.8 g).

¹H NMR (400 MHz, D₆-DMSO) δ 8.90 (s, 2H), 3.52 (t, 2H), 2.94-2.84 (m, 2H), 2.60-2.53 (m, 4H), 1.89-1.81 (m, 2H).

### c) 2-[3-(Methyl-phenethyl-amino)-propylsulfanyl]-ethanol

2-(3-Methylamino-propylsulfanyl)-ethanol hydrochloride prepared as described in Example 12b) (2.5 g) was converted to the free base using a solid phase extraction with a sulfonic acid sorbent, yielding 1.68 g. A solution of this material (800 mg) in dichloromethane (20 mL) was treated with phenylacetaldehyde (708 mg) and acetic acid (483 mg), followed by sodium triacetoxyborohydride (2.28 g) and the mixture was stirred for 3 hours at room temperature. The reaction mixture was then partitioned between dichloromethane and saturated aqueous sodium bicarbonate solution and the organic layer was extracted with dilute aqueous hydrochloric acid. Excess solid sodium bicarbonate was added to the acidic washing and the mixture was extracted with fresh dichloromethane. The organic layer was dried over magnesium sulphate and the solvent was removed under reduced pressure. The crude product was purified by flash chromatography on silica gel eluting with 1% triethylamine and 4% methanol in dichloromethane to yield the sub-titled compound (0.8 g).

m/e 254 (M+H⁺, 100%)

### d) [3-(Methyl-phenethyl-amino)-propylsulfanyl]-acetaldehyde

To a solution of 2-[3-(methyl-phenethyl-amino)-propylsulfanyl]-ethanol (Example 12c), 253 mg) and triethylamine (303 mg) in dichloromethane (3 mL), cooled to −10° C., was added in one portion a solution of sulphur trioxide-pyridine complex (477 mg) in dimethylsulphoxide (3 mL). The cooling bath was removed and the mixture was stirred vigorously for 10 minutes. The reaction mixture was then partitioned between ethyl acetate (50 mL) and aqueous phosphate buffer (50 mL, pH 7.2), the organic layer was washed with aqueous brine and the solvent was evaporated under reduced pressure to yield the sub-titled compound (170 mg).

m/e 252 (M+H⁺, 100%)

### e) 4-Hydroxy-7-((1R)-1-hydroxy-2-{[2-({3-[methyl(2-phenylethyl)amino]propyl}thio)ethyl]amino}ethyl)-1,3-benzothiazol-2(3H)-one trifluoroacetate

The titled compound was prepared from 7-[(1R)-2-amino-1-hydroxyethyl]-4-hydroxy-1,3-benzothiazol-2(3H)-one hydrochloride (Example 4b), 100 mg) and [3-(methyl-phenethylamino)-propylsulfanyl]-acetaldehyde (Example 12d), 107 mg) according to the method of Example 5e). The crude product was purified by reverse phase HPLC using a gradient elution of 5% to 75% acetonitrile in 0.2% aqueous trifluoroacetic acid to yield the titled compound (32 mg).

m/e 462 (M+H⁺, 100%)

¹H NMR (400 MHz, D₆-DMSO) δ 11.64 (s, 1H), 10.22 (s, 1H), 9.74 (s, 1H), 8.83 (s, 1H), 8.64 (s, 1H), 7.33-7.22 (m, 5H), 6.88 (d, 1H), 6.74 (d, 1H), 6.46 (s, 1H), 4.87-4.84 (m, 1H), 3.40-2.90 (m, 11H), 2.82 (s, 3H), 2.80-2.73 (m, 2H), 2.57 (t, 2H), 1.91-1.87 (m, 2H).

### EXAMPLE 13

### tert-Butyl [2-(4-ethylphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

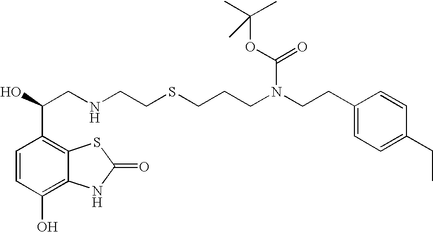

### a) tert-Butyl allyl[2-(4-ethylphenyl)ethyl]carbamate

To a solution of tert-butyl [2-(4-ethylphenyl)ethyl]carbamate (4.77 g) in dry THF (40 ml) was added 60% sodium hydride (0.84 g) under nitrogen and the mixture heated at 50° C. for 45 minutes. The solution was cooled to room temperature, allyl bromide (1.82 ml) added and the mixture stirred for 3 hours. The reaction mixture was quenched with water, extracted into ethyl acetate, dried over magnesium sulphate, filtered and concentrated. The crude product was purified flash chromatography on silica gel, eluting with 5% ethyl acetate in iso-hexane to yield the sub-titled compound (3.30 g).

¹H NMR δ(CDCL3) 7.15-7.06 (m, 4H), 5.85-5.68 (m, 1H), 5.12 (d, 2H), 3.87-3.70 (m, 2H), 3.42-3.31 (m, 2H), 2.84-2.73 (m, 2H), 2.62 (q, 2H), 1.45 (s, 9H), 1.22 (t, 3H)

### b) tert-Butyl [2-(4-ethylphenyl)ethyl]{3-[(2-hydroxyethyl)thio]propyl}carbamate

The sub-titled compound was prepared from tert-butyl allyl[2-(4-ethylphenyl)ethyl]carbamate (Example 13a), 3.3 g) according to the method of Example 10a). The crude product was purified flash chromatography on silica gel, eluting with 50% ethyl acetate in iso-hexane to yield the sub-title compound (3.91 g).

¹H NMR δ(CDCL3) 7.16-7.05 (m, 4H), 3.71 (q, 2H), 3.41-3.32 (m, 2H), 3.31-3.16 (m, 2H), 2.83-2.74 (m, 2H), 2.71 (t, 2H), 2.62 (q, 2H), 2.50 (t, 2H), 1.83-1.73 (m, 2H), 1.44 (s, 9H), 1.22 (t, 3H)

### c) tert-Butyl [2-(4-ethylphenyl)ethyl]{3-[(2-oxoethyl)thio]propyl}carbamate

The sub-titled compound was prepared from tert-Butyl [2-(4-ethylphenyl)ethyl]{3-[(2-hydroxyethyl)thio]propyl}carbamate (Example 13b), 200 mg) according to the method of Example 1b). The crude product was purified flash chromatography on silica gel, eluting with 20% ethyl acetate in iso-hexane to yield the sub-title compound (106 mg).

m/e 266 ((M−BOC)+H+, 100%)

### d) tert-Butyl [2-(4-ethylphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

The titled compound was prepared from 7-[(1R)-2-Amino-1-hydroxyethyl]-4-hydroxy-1,3-benzothiazol-2(3H)-one hydrochloride (Example 4b), 76 mg) and tert-Butyl [2-(4-ethylphenyl)ethyl]{3-[(2-oxoethyl)thio]propyl}carbamate (Example 13c), 106 mg) according to the method of Example 5e). The crude product was purified by flash chromatography on silica gel, eluting with 1% concentrated aqueous ammonia and 10% methanol in dichloromethane to yield the titled compound (95 mg).

m/e 576 (M+H+, 100%)

### EXAMPLE 14

### 7-[(1R)-2-({2-[(3-{[2-(4-Ethylphenyl)ethyl]amino}propyl)thio]ethyl}amino)-1-hydroxyethyl]-4-hydroxy-1,3-benzothiazol-2(3H)-one

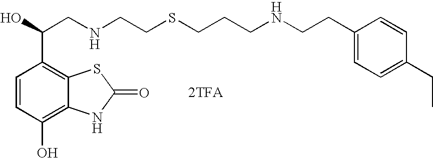

The titled compound was prepared from tert-butyl [2-(4-ethylphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 13d), 95 mg) according to the method of Example 11. The residue was purified by reverse phase HPLC using a gradient elution of 5% to 75% acetonitrile in aqueous trifluoroacetic acid to yield the titled compound (28 mg)

m/e 476 (M+H+, 100%)

¹H NMR δ(DMSO) 11.68 (s, 1H), 10.24 (s, 1H), 8.88-8.76 (m, 1H), 8.73-8.55 (m, 3H), 7.17 (4H, s), 6.92 (d, 1H), 6.77 (d, 1H), 6.48 (s, 1H), 4.93-4.85 (m, 1H), 3.21-2.96 (m, 8H), 2.90-2.71 (m, 4H), 2.66-2.53 (m, 4H), 1.86 (quintet, 2H), 1.16 (t, 3H)

### EXAMPLE 15

### tert-Butyl [2-(4-ethoxyphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

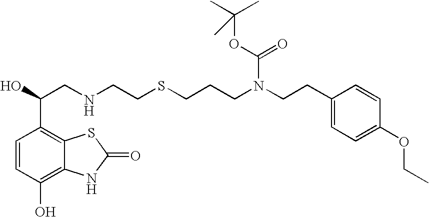

### a) tert-Butyl allyl[2-(4-ethoxyphenyl)ethyl]carbamate

The sub-titled compound was prepared from tert-butyl [2-(4-ethoxyphenyl)ethyl]carbamate (5.0 g) according to the method of Example 13a). The crude product was purified flash chromatography on silica gel, eluting with 5% ethyl acetate in iso-hexane to yield the sub-titled compound (3.01 g).

¹H NMR δ(CDCL3) 7.12-7.02 (m, 2H), 6.81 (d, 2Hd), 5.81-5.67 (m, 1H), 5.14-5.04 (m, 2H), 4.00 (q, 2H), 3.81-3.64 (m, 2H), 3.39-3.29 (m, 2H), 2.79-2.70 (m, 2H), 1.45 (s, 9H), 1.40 (t, 3H)

### b) tert-Butyl [2-(4-ethoxyphenyl)ethyl]{3-[(2-hydroxyethyl)thio]propyl}carbamate

The sub-titled compound was prepared from tert-butyl allyl[2-(4-ethoxyphenyl)ethyl]carbamate (Example 15a), 3.3 g) according to the method of Example 10a). The crude product was purified flash chromatography on silica gel, eluting with 50% ethyl acetate in iso-hexane to yield the sub-titled compound (3.87 g).

¹H NMR δ(CDCL3) 7.13-7.03 (m, 2H), 6.84-6.79 (m, 2H), 4.01 (q, 2H), 3.76-3.66 (m, 2H), 3.38-3.29 (m, 2H), 3.28-3.13 (m, 2H), 2.79-2.69 (m, 4H), 2.52-2.45 (m, 2H), 1.81-1.72 (m, 2H), 1.45 (s, 9H), 1.40 (t, 3H)

### c) tert-Butyl [2-(4-ethoxyphenyl)ethyl]{3-[(2-oxoethyl)thio]propyl}carbamate

The sub-titled compound was prepared from tert-butyl [2-(4-ethoxyphenyl)ethyl]{3-[(2-hydroxyethyl)thio]propyl}carbamate (Example 15b), 600 mg) according to the method of Example 1b). The crude product was purified flash chromatography on silica gel, eluting with 20% ethyl acetate in iso-hexane to yield the sub-titled compound (170 mg).

m/e 282 ((M−BOC)+H+, 100%)

### d) tert-Butyl [2-(4-ethoxyphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

The titled compound was prepared from 7-[(1R)-2-Amino-1-hydroxyethyl]-4-hydroxy-1,3-benzothiazol-2(3H)-one hydrochloride (Example 4b), 117 mg) and tert-Butyl [2-(4-ethylphenyl)ethyl]{3-[(2-oxoethyl)thio]propyl}carbamate (Example 15c), 170 mg) according to the method of Example 5e). The crude product was purified by flash chromatography on silica gel, eluting with 1% concentrated aqueous ammonia and 10% methanol in dichloromethane to yield the titled compound (120 mg).

m/e 592 (M+H+, 100%)

### EXAMPLE 16

### 7-[(1R)-2-({2-[(3-{[2-(4-Ethoxyphenyl)ethyl]amino}propyl)thio]ethyl}amino)-1-hydroxyethyl]-4-hydroxy-1,3-benzothiazol-2(3H)-one

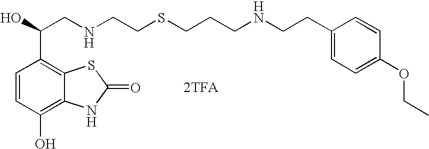

The titled compound was prepared from tert-butyl [2-(4-ethoxyphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 15d), 120 mg) according to the method of Example 11. The residue was purified by reverse phase HPLC using a gradient elution of 5% to 75% acetonitrile in aqueous trifluoroacetic acid to yield the titled compound (49 mg)

m/e 492 (M+H+, 100%)

¹H NMR δ(DMSO) 11.67 (s, 1H), 10.25 (s, 1H), 8.89-8.77 (m, 1H), 8.72-8.55 (m, 3H), 7.16 (d, 2H), 6.94-6.86 (m, 3H), 6.77 (d, 1H), 6.49 (s, 1H), 4.92-4.86 (m, 1H), 3.99 (q, 2H), 3.20-2.98 (m, 8H), 2.86-2.73 (m, 4H), 2.62 (t, 2H), 1.86 (quintet, 2H), 1.31 (t, 3H)

### EXAMPLE 17

### tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}{2-[3-(trifluoromethyl)phenyl]ethyl}carbamate

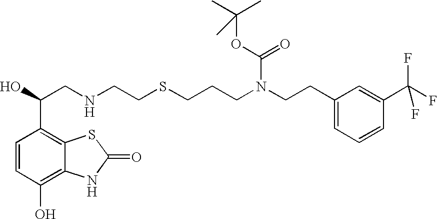

### a) tert-Butyl allyl{2-[3-(trifluoromethyl)phenyl]ethyl}carbamate

The sub-titled compound was prepared from tert-butyl {2-[3-(trifluoromethyl)phenyl]ethyl}carbamate (3.11 g) according to the method of Example 13a). The crude product was purified flash chromatography on silica gel, eluting with 5% ethyl acetate in iso-hexane to yield the sub-titled compound (1.39 g).

¹H NMR δ(CDCL3) 7.50-7.31 (m, 4H), 5.74 (s, 1H), 5.12 (s, 2H), 3.88-3.65 (m, 2H), 3.46-3.35 (m, 2H), 2.88 (s, 2H), 1.46 (9H, t)

### b) tert-Butyl {3-[(2-hydroxyethyl)thio]propyl}{2-[3-(trifluoromethyl)phenyl]ethyl}carbamate

The sub-titled compound was prepared from tert-butyl allyl{2-[3-(trifluoromethyl)phenyl]ethyl}carbamate (Example 17a), 1.39 g) according to the method of Example 10a). The crude product was purified flash chromatography on silica gel, eluting with 50% ethyl acetate in iso-hexane to yield the sub-titled compound (1.23 g).

¹H NMR δ(CDCL3) 7.55-7.31 (m, 4H), 3.71 (quintet, 2H), 3.45-3.37 (m, 2H), 3.32-3.12 (m, 2H), 2.94-2.83 (m, 2H), 2.72 (t, 2H), 2.54-2.45 (m, 2H), 1.85-1.71 (m, 2H), 1.43 (s, 9H)

### c) tert-Butyl {3-[(2-oxoethyl)thio]propyl}{2-[3-(trifluoromethyl)phenyl]ethyl}carbamate

To a solution of tert-butyl {3-[(2-hydroxyethyl)thio]propyl}{2-[3-(trifluoromethyl)phenyl]ethyl}carbamate (Example 17b), 600 mg) in dichloromethane (12 ml) was added Dess-Martin periodinane (624 mg) and the mixture stirred under nitrogen for one hour. It was washed with dilute sodium hydroxide solution, washed with water, dried over anhydrous magnesium sulphate, filtered and concentrated. The crude product was purified flash chromatography on silica gel, eluting with 20% ethyl acetate in iso-hexane to yield the sub-titled compound (390 mg).

m/e 404 (M−H−, 100%)

### d) tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}{2-[3-(trifluoromethyl)phenyl]ethyl}carbamate

The titled compound was prepared from 7-[(1R)-2-Amino-1-hydroxyethyl]-4-hydroxy-1,3-benzothiazol-2(3H)-one hydrochloride (Example 4b), 250 mg) and tert-butyl {3-[(2-oxoethyl)thio]propyl}{2-[3-(trifluoromethyl)phenyl]ethyl}carbamate (Example 17c), 390 mg) according to the method of Example 5e). The crude product was purified by flash chromatography on silica gel, eluting with 1% concentrated aqueous ammonia and 10% methanol in dichloromethane to yield the titled compound (223 mg).

m/e 616 (M+H+, 100%)

### EXAMPLE 18

### 4-Hydroxy-7-{(1R)-1-hydroxy-2-[(2-{[3-({2-[3-(trifluoromethyl)phenyl]ethyl}amino)propyl]thio}ethyl)amino]ethyl}-1,3-benzothiazol-2(3H)-one

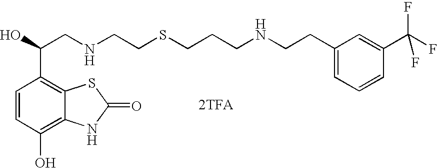

The titled compound was prepared from tert-butyl {3-[(2-{[(2R)-2-hydroxy-2-(4-hydroxy-2-oxo-2,3-dihydro-1,3-benzothiazol-7-yl)ethyl]amino}ethyl)thio]propyl}{2-[3-(trifluoromethyl)phenyl]ethyl}carbamate (Example 17d), 223 mg) according to the method of Example 11. The residue was purified by reverse phase HPLC using a gradient elution of 23% to 29% acetonitrile in aqueous trifluoroacetic acid to yield the titled compound (63 mg)

m/e 516 (M+H+, 100%)

¹H NMR δ(DMSO) 11.67 (s, 1H), 10.25 (s, 1H), 8.85 (s, 1H), 8.70 (s, 3H), 7.68-7.58 (m, 4H), 6.92 (d, 1H), 6.77 (d, 1H), 6.49 (s, 1H), 4.93-4.85 (m, 1H), 3.30-2.97 (m, 10H), 2.84-2.72 (m, 2H), 2.63 (t, 2H), 1.87 (quintet, 2H)

### EXAMPLE 19

### tert-Butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

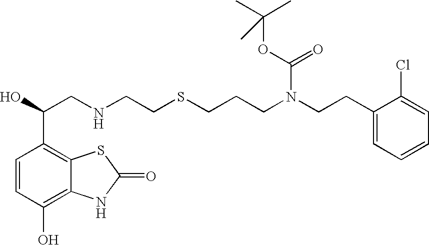

### a) tert-Butyl allyl[2-(2-chlorophenyl)ethyl]carbamate

The sub-titled compound was prepared from tert-butyl [2-(2-chlorophenyl)ethyl]carbamate (4.61 g) according to the method of Example 13a). The crude product was purified flash chromatography on silica gel, eluting with 5% ethyl acetate in iso-hexane to yield the sub-titled compound (2.58 g).

¹H NMR δ(CDCL3) 7.38-7.30 (m, 1H), 7.23-7.13 (m, 3H), 5.86-5.67 (m, 1H), 5.19-5.05 (m, 2H), 3.88-3.66 (m, 2H), 3.46-3.38 (m, 2H), 3.04-2.90 (m, 2H), 1.43 (s, 9H) c; b) tert-Butyl [2-(2-chlorophenyl)ethyl]{3-[(2-hydroxyethyl)thio]propyl}carbamate

The sub-titled compound was prepared from tert-butyl allyl[2-(2-chlorophenyl)ethyl]carbamate (Example 19a), 2.00 g) according to the method of Example 10a). The crude product was purified flash chromatography on silica gel, eluting with 50% ethyl acetate in iso-hexane to yield the sub-titled compound (2.22 g).

¹H NMR δ_{(CDCL3)} 7.37-7.31 (m, 1H), 7.22-7.12 (m, 3H), 3.71 (q, 2H), 3.41 (t, 2H), 3.33-3.13 (m, 2H), 3.03-2.91 (m, 2H), 2.72 (t, 2H), 2.55-2.36 (m, 2H), 1.86-1.71 (m, 2H), 1.42 (s, 9H)

### c) tert-Butyl [2-(2-chlorophenyl)ethyl]{3-[(2-oxoethyl)thio]propyl}carbamate

The sub-titled compound was prepared from tert-butyl [2-(2-chlorophenyl)ethyl]{3-[(2-hydroxyethyl)thio]propyl}carbamate (Example 19b), 600 mg) according to the method of Example 17c). The crude product was purified flash chromatography on silica gel, eluting with 20% ethyl acetate in iso-hexane to yield the sub-titled compound (240 mg).

m/e 272 ((M−BOC)+H+, 100%)

### d) tert-Butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

The titled compound was prepared from 7-[(1R)-2-Amino-1-hydroxyethyl]-4-hydroxy-1,3-benzothiazol-2(3H)-one hydrochloride (Example 4b), 170 mg) and tert-butyl [2-(2-chlorophenyl)ethyl]{3-[(2-oxoethyl)thio]propyl}carbamate (Example 19c), 240 mg) according to the method of Example 5e). The crude product was purified by flash chromatography on silica gel, eluting with 1% concentrated aqueous ammonia and 10% methanol in dichloromethane to yield the titled compound (157 mg).

m/e 582 (M+H+, 100%)

### EXAMPLE 20

### 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one

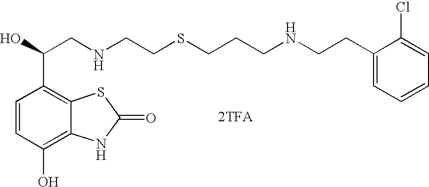

The titled compound was prepared from tert-butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 19d), 157 mg) according to the method of Example 11. The residue was purified by reverse phase HPLC using a gradient elution of 23% to 25% acetonitrile in aqueous trifluoroacetic acid to yield the titled compound (150 mg)

m/e 482 (M+H+, 100%)

¹H NMR δ(DMSO) 11.67 (s, 1H), 10.27 (s, 1H), 8.90-8.63 (m, 4H), 7.49-7.46 (m, 1H), 7.41-7.30 (m, 3H), 6.92 (d, 1H), 6.77 (d, 1H), 6.49 (s, 1H), 4.92-4.87 (m, 1H), 3.20-3.02 (m, 10H), 2.86-2.72 (m, 2H), 2.63 (t, 2H), 1.88 (quintet, 3H)

### EXAMPLE 21

### tert-Butyl ((1S)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate

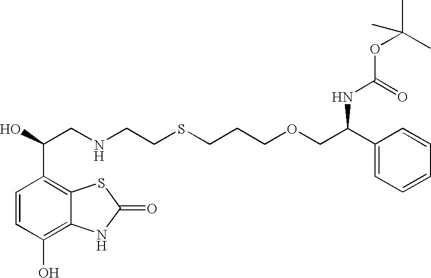

### a) tert-Butyl ((1S)-2-{3-[(2-hydroxyethyl)thio]propoxy}-1-phenylethyl)carbamate

The sub-titled compound was prepared from tert-butyl [(1S)-2-(allyloxy)-1-phenylethyl]carbamate (1.00 g) according to the method of Example 10a). The crude product was purified flash chromatography on silica gel, eluting with 50% ethyl acetate in iso-hexane to yield the sub-titled compound (1.28 g).

¹H NMR δ(CDCL3) 7.36-7.22 (5H, m), 5.32 (d, 1H), 4.81 (s, 1H), 3.74-3.38 (m, 6H), 2.69 (t, 2H), 2.56 (t, 2H), 1.82 (quintet, 2H), 1.41 (s, 9H)

### b) tert-Butyl ((1S)-2-{3-[(2-oxoethyl)thio]propoxy}-1-phenylethyl)carbamate

The sub-titled compound was prepared from tert-butyl ((1S)-2-{3-[(2-hydroxyethyl)thio]propoxy}-1-phenylethyl)carbamate (Example 21b), 540 mg) according to the method of Example 17c). The crude product was purified flash chromatography on silica gel, eluting with 20% ethyl acetate in iso-hexane to yield the sub-titled compound (330 mg).

m/e 254 ((M−BOC)+H+, 100%)

### c) tert-Butyl ((1S)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate

The titled compound was prepared from 7-[(1R)-2-Amino-1-hydroxyethyl]-4-hydroxy-1,3-benzothiazol-2(3H)-one hydrochloride (Example 4b), 245 mg) and tert-butyl ((1S)-2-{3-[(2-oxoethyl)thio]propoxy}-1-phenylethyl)carbamate (Example 21b), 330 mg) according to the method of Example 5e). The crude product was purified by flash chromatography on silica gel, eluting with 1% concentrated aqueous ammonia and 10% methanol in dichloromethane to yield the titled compound (207 mg).

m/e 564 (M+H+, 100%)

### EXAMPLE 22

### 7-((1R)-2-{[2-({3-[(2S)-2-Amino-2-phenylethoxy]propyl}thio)ethyl]amino}-1-hydroxyethyl)-4-hydroxy-1,3-benzothiazol-2(3H)-one

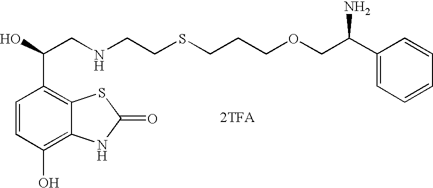

The titled compound was prepared from tert-butyl ((1S)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate (Example 21c), 207 mg) according to the method of Example 11. The residue was purified by reverse phase HPLC using a gradient elution of 9% to 13% acetonitrile in aqueous trifluoroacetic acid to yield the titled compound (90 mg)

m/e 464 (M+H+, 100%)

¹H NMR δ(DMSO) 11.69 (s, 1H), 10.28 (s, 1H), 8.87 (s, 1H), 8.68 (s, 1H), 8.45 (s, 3H), 7.52-7.38 (m, 5H), 6.94 (d, 1H), 6.79 (d, 1H), 6.50 (s, 1H), 4.94-4.87 (m, 1H), 4.55-4.45 (m, 1H), 3.71-3.65 (m, 2H), 3.62-3.48 (m, 2H), 3.20-3.03 (m, 4H), 2.86-2.68 (m, 2H), 2.59 (t, 2H), 1.80 (quintet, 2H)

### EXAMPLE 23

### tert-Butyl ((1R)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate

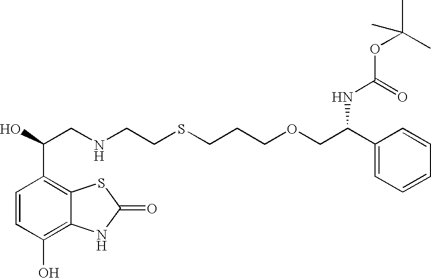

### a) tert-Butyl ((1R)-2-{3-[(2-hydroxyethyl)thio]propoxy}-1-phenylethyl)carbamate

The sub-titled compound was prepared from tert-butyl [(1R)-2-(allyloxy)-1-phenylethyl]carbamate (1.00 g) according to the method of Example 10a). The crude product was purified flash chromatography on silica gel, eluting with 50% ethyl acetate in iso-hexane to yield the sub-titled compound (1.23 g).

¹H NMR δ(CDCL3) 7.36-7.22 (m, 5H), 5.32 (d, 1H), 4.81 (s, 1H), 3.74-3.38 (m, 6H), 2.69 (t, 2H), 2.56 (t, 2H), 1.82 (quintet, 2H), 1.41 (s, 9H)

### b) tert-Butyl ((1R)-2-{3-[(2-oxoethyl)thio]propoxy}-1-phenylethyl)carbamate

The sub-titled compound was prepared from tert-butyl ((1R)-2-{3-[(2-hydroxyethyl)thio]propoxy}-1-phenylethyl)carbamate (Example 23b), 500 mg) according to the method of Example 17c). The crude product was purified flash chromatography on silica gel, eluting with 20% ethyl acetate in iso-hexane to yield the sub-titled compound (143 mg).

m/e 254 ((M−BOC)+H+, 100%)

### c) tert-Butyl ((1R)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate

The titled compound was prepared from 7-[(1R)-2-Amino-1-hydroxyethyl]-4-hydroxy-1,3-benzothiazol-2(3H)-one hydrochloride (Example 4b), 106 mg) and tert-butyl ((1R)-2-{3-[(2-oxoethyl)thio]propoxy}-1-phenylethyl)carbamate (Example 23b), 143 mg) according to the method of Example 5e). The crude product was purified by flash chromatography on silica gel, eluting with 1% concentrated aqueous ammonia and 10% methanol in dichloromethane to yield the titled compound (100 mg).

m/e 564 (M+H+, 100%)

### EXAMPLE 24

### 7-((1R)-2-{[2-({3-[(2R)-2-Amino-2-phenylethoxy]propyl}thio)ethyl]amino}-1-hydroxyethyl)-4-hydroxy-1,3-benzothiazol-2(3H)-one

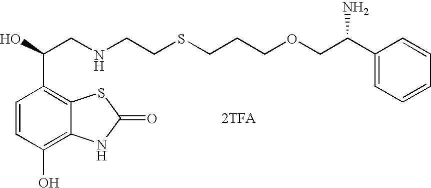

The titled compound was prepared from tert-butyl ((1R)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate (Example 23c), 100 mg) according to the method of Example 11. The residue was purified by reverse phase HPLC using a gradient elution of 9% to 13% acetonitrile in aqueous trifluoroacetic acid to yield the titled compound (43 mg)

m/e 464 (M+H+, 100%)

¹H NMR δ(DMSO) 11.67 (s, 1H), 10.26 (s, 1H), 8.85 (s, 1H), 8.66 (s, 1H), 8.43 (s, 3H), 7.50-7.37 (m, 5H), 6.92 (d, 1H), 6.77 (d, 1H), 6.48 (s, 1H), 4.92-4.85 (m, 1H), 4.48 (s, 1H), 3.67 (d, 2H), 3.61-3.46 (m, 2H), 3.19-3.01 (m, 4H), 2.83-2.68 (m, 2H), 2.57 (t, 2H), 1.78 (quintet, 2H)

### EXAMPLE 25

### 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one

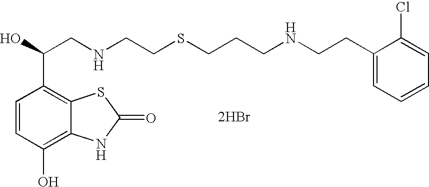

The titled compound was prepared from tert-butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 19d), 5.93 g) according to the method of Example 11. The residue was purified by reverse phase HPLC using a gradient elution of 5% to 50% acetonitrile in aqueous ammonium acetate. The diacetate salt was dissolved in water, basified with aqueous ammonia, the supernatant decanted and the residue dried under high vacuum. It was dissolved in ethanol, acidified with 48% HBr, and the solid collected by filtration to yield the titled compound (4.53 g)

m/e 482 (M+H+, 100%)

¹H NMR δ(DMSO) 11.67 (s, 1H), 10.15 (s, 1H), 8.70 (s, 4H), 7.50-7.30 (m, 4H), 6.94 (d, 1H), 6.78 (d, 1H), 6.45 (s, 1H), 4.96-4.90 (m, 1H), 3.22-3.02 (m, 10H), 2.86-2.76 (m, 2H), 2.66 (t, 2H), 1.91 (quintet, 2H)

### EXAMPLE 26

### tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2R)-2-phenylpropyl]carbamate

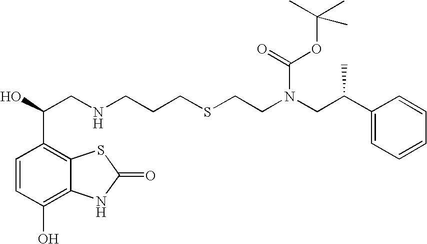

### a) Methyl 3-[(2-oxo-2-{[(2R)-2-phenylpropyl]amino}ethyl)thio]propanoate

To a solution of [(3-methoxy-3-oxopropyl)thio]acetic acid (1.17 g) in dichloromethane (25 ml) was added oxalyl chloride (1.14 ml) and dimethylformamide (50 μl) and the resulting mixture stirred at room temperature for 2 hours. It was concentrated in vacuo, dissolved in dichloromethane (15 ml) and added to a solution of [(2R)-2-phenylpropyl]amine (0.93 ml) and diisopropylethylamine (1.08 ml) in dichloromethane (20 ml) and the mixture stirred at room temperature for 18 hours. It was washed successively with dilute hydrochloric acid, sodium bicarbonate solution, and water, dried over magnesium sulphate, filtered and concentrated to yield the sub-titled compound (1.93 g).

m/e 296 (M+H+, 100%)

### b) 3-[(2-Oxo-2-{[(2R)-2-phenylpropyl]amino}ethyl)thio]propanoic acid

To a solution of methyl 3-[(2-oxo-2-{[(2R)-2-phenylpropyl]amino}ethyl)thio]propanoate (Example Ba), 1.93 g) in tetrahydrofuran (40 ml) was added 1M lithium hydroxide solution (14.4 ml) and the reaction mixture stirred at room temperature for 18 hours. It was concentrated in vacuo, the residue diluted with water, acidified with dilute hydrochloric acid, and extracted into ethyl acetate. It was washed with water, dried over magnesium sulphate, filtered and concentrated to yield the sub-titled compound (1.83 g).

¹H NMR δ(CDCL3) 7.36-7.29 (m, 2H), 7.27-7.20 (m, 3H), 6.85-6.78 (m, 1H), 3.62 (d, 1H), 3.35 (ddd, 1H), 3.19 (s, 2H), 3.05-2.95 (m, 1H), 2.60-2.49 (m, 4H), 1.29 (d, 3H)

### c) 3-[(2-{[(2R)-2-Phenylpropyl]amino}ethyl)thio]propan-1-ol

To a solution of 3-[(2-oxo-2-{[(2R)-2-phenylpropyl]amino}ethyl)thio]propanoic acid (Example Bb), 1.83 g) in tetrahydrofuran (20 ml) was added 1M borane tetrahydrofuran complex (26.2 ml) and the reaction mixture heated under reflux for 1.5 hours. It was cooled, carefully quenched with methanol, and concentrated in vacuo. It was dissolved in methanol, acidified with concentrated hydrochloric acid (4 ml) and heated under reflux for 15 minutes. It was concentrated in vacuo, diluted with ethyl acetate, and extracted into water. The aqueous extract was basified with sodium hydroxide solution, and the product extracted into ethyl acetate, dried over magnesium sulphate, filtered and concentrated to yield the sub-titled compound (1.27 g).

¹H NMR δ(CDCL3) 7.34-7.29 (m, 2H), 7.25-7.19 (m, 3H), 3.71 (t, 2H), 2.94 (sextet, 1H), 2.85-2.72 (m, 4H), 2.65-2.55 (m, 4H), 1.78 (quintet, 2H), 1.29-1.25 (m, 3H)

### d) tert-Butyl {2-[(3-hydroxypropyl)thio]ethyl}[(2R)-2-phenylpropyl]carbamate

To a solution of 3-[(2-{[(2R)-2-phenylpropyl]amino}ethyl)thio]propan-1-ol (Example Bc), 1.27 g) and triethylamine (0.7 ml) in tetrahydrofuran (30 ml) was added di-tert-butyl dicarbonate (1.09 g) and the reaction mixture stirred at room temperature for 24 hours. It was concentrated in vacuo to yield the sub-titled compound (1.77 g).

m/e 254 ((M−BOC)+H+, 100%)

¹H NMR δ(CDCL3) 7.33-7.27 (m, 2H), 7.24-7.15 (m, 3H), 3.78-3.69 (m, 2H), 3.60-2.91 (m, 5H), 2.68-2.31 (m, 4H), 1.88-1.77 (m, 2H), 1.43 (s, 9H), 1.25 (d, 3H)

### e) tert-Butyl {2-[(3-oxopropyl)thio]ethyl}[(2R)-2-phenylpropyl]carbamate

The sub-titled compound was prepared from tert-butyl {2-[(3-hydroxypropyl)thio]ethyl}[(2R)-2-phenylpropyl]carbamate (Example 26d), 400 mg) according to the method of Example 9b). The crude product was purified flash chromatography on silica gel, eluting with 20% ethyl acetate in iso-hexane to yield the sub-titled compound (280 mg).

¹H NMR δ(CDCL3) 9.75 (t, 1H), 7.34-7.26 (m, 2H), 7.25-7.15 (m, 3H), 3.65-2.88 (5H, m), 2.82-2.28 (m, 6H), 1.43 (s, 9H), 1.25 (d, 3H)

### f) tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2R)-2-phenylpropyl]carbamate

The titled compound was prepared from 7-[(1R)-2-Amino-1-hydroxyethyl]-4-hydroxy-1,3-benzothiazol-2(3H)-one hydrochloride (Example 4b), 150 mg) and tert-butyl {2-[(3-oxopropyl)thio]ethyl}[(2R)-2-phenylpropyl]carbamate (Example 26e), 201 mg) according to the method of Example 5e). The crude product was purified by flash chromatography on silica gel, eluting with 1% concentrated aqueous ammonia and 10% methanol in dichloromethane to yield the titled compound (180 mg).

m/e 562 (M+H+, 100%)

### EXAMPLE 27

### 4-Hydroxy-7-[(1R)-1-hydroxy-2-({3-[(2-{[(2R)-2-phenylpropyl]amino}ethyl)thio]propyl}amino)ethyl]-1,3-benzothiazol-2(3H)-one

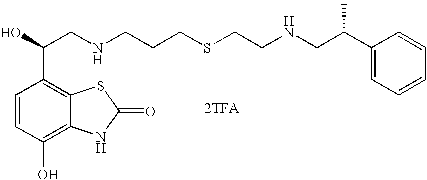

A solution of tert-butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2R)-2-phenylpropyl]carbamate (Example Bf), 180 mg) in trifluoroacetic acid (3 ml) and dichloromethane (3 ml) was stirred at room temperature for 30 minutes. It was diluted with toluene (10 ml) and concentrated in vacuo. The residue was purified by reverse phase HPLC using a gradient elution of 10% to 30% acetonitrile in aqueous trifluoroacetic acid to yield the titled compound (44 mg)

m/e 462 (M+H+, 100%).

¹H NMR δ(DMSO) 11.67 (s, 1H), 10.25 (s, 1H), 8.81-8.60 (m, 3H), 8.42 (s, 1H), 7.39-7.33 (m, 2H), 7.32-7.25 (m, 3H), 6.93 (d, 1H), 6.77 (d, 1H), 6.48 (s, 1H), 4.87 (dd, 1H), 3.21-2.97 (m, 9H), 2.74 (t, 2H), 2.59 (t, 2H), 1.88 (sextet, 2H), 1.27 (d, 3H)

### EXAMPLE 28

### tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2S)-2-phenylpropyl]carbamate

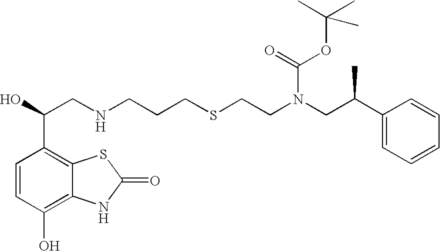

### a) Methyl 3-[(2-oxo-2-{[(2S)-2-phenylpropyl]amino}ethyl)thio]propanoate

The sub-titled compound was prepared from [(3-methoxy-3-oxopropyl)thio]acetic acid (1.17 g) and [(2S)-2-phenylpropyl]amine (0.93 ml) according to the method of Example 26a) to yield the sub-titled compound (1.93 g).

m/e 296 (M+H+, 100%)

### b) 3-[(2-Oxo-2-{[(2S)-2-phenylpropyl]amino}ethyl)thio]propanoic acid

The sub-titled compound was prepared from methyl 3-[(2-oxo-2-{[(2S)-2-phenylpropyl]amino}ethyl)thio]propanoate (Example 28a), 1.93 g) according to the method of Example 26b) to yield the sub-titled compound (1.83 g).

m/e 282 (M+H+, 100%)

¹H NMR δ(CDCL3) 7.36-7.30 (m, 2H), 7.27-7.20 (m, 3H), 6.82-6.76 (m, 1H), 3.62 (dt, 1H), 3.40-3.30 (m, 1H), 3.19 (s, 2H), 3.05-2.95 (m, 1H), 2.60-2.50 (m, 4H), 1.29 (d, 3H)

### c) 3-[(2-{[(2S)-2-Phenylpropyl]amino}ethyl)thio]propan-1-ol

The sub-titled compound was prepared from 3-[(2-oxo-2-{[(2S)-2-phenylpropyl]amino}ethyl)thio]propanoic acid (Example 28b), 1.83 g) according to the method of Example 26c) to yield the sub-titled compound (1.19 g).

¹H NMR δ(CDCL3) 7.35-7.28 (m, 2H), 7.24-7.19 (m, 3H), 3.71 (t, 2H), 2.94 (sextet, 1H), 2.85-2.72 (m, 4H), 2.65-2.55 (m, 4H), 1.82-1.74 (m, 2H), 1.27 (d, 3H)

### d) tert-Butyl {2-[(3-hydroxypropyl)thio]ethyl}[(2S)-2-phenylpropyl]carbamate

The sub-titled compound was prepared from 3-[(2-{[(2S)-2-phenylpropyl]amino}ethyl)thio]propan-1-ol (Example 28c), 1.19 g) according to the method of Example 26d) to yield the sub-titled compound (1.66 g).

m/e 254 ((M−BOC)+H+, 100%)

¹H NMR δ(CDCL3) 7.34-7.27 (m, 2H), 7.24-7.14 (m, 3H), 3.77-3.69 (m, 2H), 3.61-2.91 (5H, m), 2.68-2.33 (m, 4H), 1.88-1.77 (m, 2H), 1.43 (s, 9H), 1.25 (d, 3H)

### e) tert-Butyl {2-[(3-oxopropyl)thio]ethyl}[(2S)-2-phenylpropyl]carbamate

The sub-titled compound was prepared from tert-butyl {2-[(3-hydroxypropyl)thio]ethyl}[(2S)-2-phenylpropyl]carbamate (Example 28d), 400 mg) according to the method of Example 9b). The crude product was purified flash chromatography on silica gel, eluting with 20% ethyl acetate in iso-hexane to yield the sub-titled compound (280 mg).

¹H NMR δ(CDCL3) 9.75 (t, 1H), 7.33-7.27 (m, 2H), 7.24-7.14 (m, 3H), 3.62-2.90 (m, 5H), 2.81-2.29 (m, 6H), 1.43 (s, 9H), 1.25 (d, 3H)

### f) tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2S)-2-phenylpropyl]carbamate

The titled compound was prepared from 7-[(1R)-2-Amino-1-hydroxyethyl]-4-hydroxy-1,3-benzothiazol-2(3H)-one hydrochloride (Example 4b), 150 mg) and tert-butyl {2-[(3-oxopropyl)thio]ethyl}[(2S)-2-phenylpropyl]carbamate (Example De), 201 mg) according to the method of Example 5e). The crude product was purified by flash chromatography on silica gel, eluting with 1% concentrated aqueous ammonia and 10% methanol in dichloromethane to yield the titled compound (180 mg).

m/e 562 (M+H+, 100%)

### EXAMPLE 29

### 4-Hydroxy-7-[(1R)-1-hydroxy-2-({3-[(2-{[(2S)-2-phenylpropyl]amino}ethyl)thio]propyl}amino)ethyl]-1,3-benzothiazol-2(3H)-one

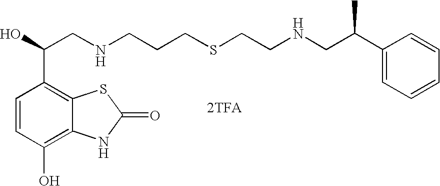

The titled compound was prepared from tert-butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2S)-2-phenylpropyl]carbamate (Example 28f), 180 mg) according to the method of Example 27. The residue was purified by reverse phase HPLC using a gradient elution of 10% to 30% acetonitrile in aqueous trifluoroacetic acid to yield the titled compound (59 mg).

m/e 462 (M+H+, 100%)

¹H NMR δ(DMSO) 11.67 (s, 1H), 10.24 (s, 1H), 8.78-8.56 (m, 3H), 8.39 (s, 1H), 7.40-7.33 (m, 2H), 7.33-7.25 (m, 3H), 6.93 (d, 1H), 6.77 (d, 1H), 6.47 (s, 1H), 4.87 (d, 1H), 3.22-2.97 (m, 9H), 2.73 (t, 2H), 2.59 (t, 2H), 1.87 (sextet, 2H), 1.27 (d, 3H)

### EXAMPLE 30

### tert-Butyl [2-(2-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate

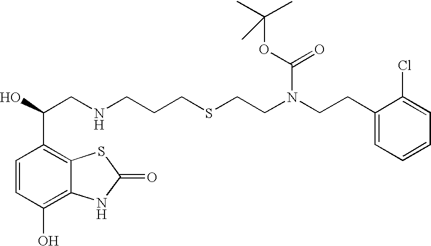

### a) Methyl 3-[(2-{[2-(2-chlorophenyl)ethyl]amino}-2-oxoethyl)thio]propanoate

The sub-titled compound was prepared from [(3-methoxy-3-oxopropyl)thio]acetic acid (1.17 g) and [2-(2-chlorophenyl)ethyl]amine (0.91 ml) according to the method of Example 26a) to yield the sub-titled compound (2.06 g).

m/e 316 (M+H+, 100%)

### b) 3-[(2-{[2-(2-Chlorophenyl)ethyl]amino}-2-oxoethyl)thio]propanoic acid

The sub-titled compound was prepared from methyl 3-[(2-{[2-(2-chlorophenyl)ethyl]amino}-2-oxoethyl)thio]propanoate (Example 30a), 2.06 g) according to the method of Example 26b) to yield the sub-titled compound (1.97 g).

m/e 302 (M+H+, 100%)

¹H NMR δ(CDCL3) 7.38-7.34 (m, 1H), 7.26-7.16 (m, 3H), 7.03-6.96 (m, 1H), 3.59 (q, 2H), 3.25 (s, 2H), 3.01 (t, 2H), 2.74 (t, 2H), 2.63 (t, 2H)

### c) 3-[(2-{[2-(2-Chlorophenyl)ethyl]amino}ethyl)thio]propan-1-ol

The sub-titled compound was prepared from 3-[(2-{[2-(2-chlorophenyl)ethyl]amino}-2-oxoethyl)thio]propanoic acid (Example 30b), 1.97 g) according to the method of Example 26c) to yield the sub-titled compound (1.00 g).

¹H NMR δ(CDCL3) 7.37-7.32 (m, 1H), 7.26-7.13 (m, 3H), 3.75 (t, 2H), 2.98-2.84 (m, 6H), 2.72-2.62 (m, 4H), 1.87-1.79 (m, 2H)

### d) tert-Butyl [2-(2-chlorophenyl)ethyl]{2-[(3-hydroxypropyl)thio]ethyl}carbamate

The sub-titled compound was prepared from 3-[(2-{[2-(2-chlorophenyl)ethyl]amino}ethyl)thio]propan-1-ol (Example 30c), 1.00 g) according to the method of Example 26d) to yield the sub-titled compound (1.37 g).

m/e 274 ((M−BOC)+H+, 100%)

¹H NMR δ(CDCL3) 7.38-7.31 (m, 1H), 7.21-7.13 (m, 3H), 3.79-3.70 (m, 2H), 3.49-3.20 (m, 4H), 3.03-2.90 (m, 2H), 2.73-2.51 (m, 4H), 1.89-1.80 (m, 2H), 1.50-1.37 (m, 9H)

### e) tert-Butyl [2-(2-chlorophenyl)ethyl]{2-[(3-oxopropyl)thio]ethyl}carbamate

The sub-titled compound was prepared from tert-butyl [2-(2-chlorophenyl)ethyl]{2-[(3-hydroxypropyl)thio]ethyl}carbamate (Example 30d), 420 mg) according to the method of Example 9b). The crude product was purified flash chromatography on silica gel, eluting with 20% ethyl acetate in iso-hexane to yield the sub-titled compound (282 mg).

¹H NMR δ(CDCL3) 9.77 (d, 1H), 7.35 (d, 1H), 7.22-7.12 (m, 3H), 3.46 (t, 2H), 3.39-3.19 (m, 2H), 3.04-2.90 (m, 2H), 2.87-2.53 (m, 6H), 1.50-1.37 (m, 9H)

### f) tert-Butyl [2-(2-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate

The titled compound was prepared from 7-[(1R)-2-Amino-1-hydroxyethyl]-4-hydroxy-1,3-benzothiazol-2(3H)-one hydrochloride (Example 4b), 150 mg) and tert-butyl [2-(2-chlorophenyl)ethyl]{2-[(3-oxopropyl)thio]ethyl}carbamate (Example 30e), 212 mg) according to the method of Example 5e). The crude product was purified by flash chromatography on silica gel, eluting with 1% concentrated aqueous ammonia and 10% methanol in dichloromethane to yield the titled compound (176 mg).

m/e 582 (M+H+, 100%)

### EXAMPLE 31

### 7-[(1R)-2-({3-[(2-{[2-(2-Chlorophenyl)ethyl]amino}ethyl)thio]propyl}amino)-1-hydroxyethyl]-4-hydroxy-1,3-benzothiazol-2(3H)-one

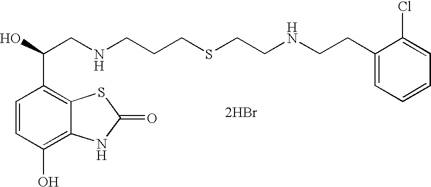

The titled compound was prepared from tert-butyl [2-(2-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate (Example 30f), 176 mg) according to the method of Example 27. The residue was dissolved in ethanol, acidified with 5% hydrobromic acid and concentrated in vacuo, not to dryness. It was triturated with acetonitrile and the solid collected by filtration to yield the titled compound (164 mg).

m/e 482 (M+H+, 100%)

¹H NMR δ(DMSO) 11.67 (s, 1H), 10.15 (s, 1H), 8.81-8.60 (m, 4H), 7.51-7.29 (m, 4H), 6.95 (d, 1H), 6.78 (d, 1H), 6.44 (d, 1H), 4.96-4.88 (m, 1H), 3.26-2.99 (m, 10H), 2.80 (t, 2H), 2.65 (t, 2H), 1.99-1.85 (m, 2H)

### EXAMPLE 32

### tert-Butyl [2-(3-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate

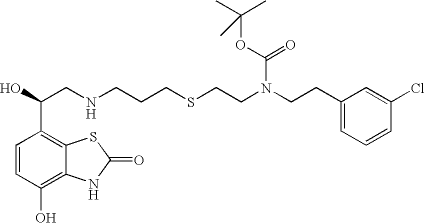

### a) Methyl 3-[(2-{[2-(3-chlorophenyl)ethyl]amino}-2-oxoethyl)thio]propanoate

The sub-titled compound was prepared from [(3-methoxy-3-oxopropyl)thio]acetic acid (1.17 g) and [2-(3-chlorophenyl)ethyl]amine (0.91 ml) according to the method of Example 26a) to yield the sub-titled compound (2.06 g).

m/e 316 (M+H+, 100%)

### b) 3-[(2-{[2-(3-Chlorophenyl)ethyl]amino}-2-oxoethyl)thio]propanoic acid

The sub-titled compound was prepared from methyl 3-[(2-{[2-(3-chlorophenyl)ethyl]amino}-2-oxoethyl)thio]propanoate (Example 32a), 2.06 g) according to the method of Example 26b) to yield the sub-titled compound (1.97 g).

m/e 302 (M+H+, 100%)

¹H NMR δ(CDCL3) 7.26-7.18 (m, 3H), 7.12-7.07 (m, 1H), 7.04-6.97 (m, 1H), 3.56 (dd, 2H), 3.25 (s, 2H), 2.84 (t, 2H), 2.75-2.69 (m, 2H), 2.66-2.60 (m, 2H)

### c) 3-[(2-{[2-(3-Chlorophenyl)ethyl]amino}ethyl)thio]propan-1-ol

The sub-titled compound was prepared from 3-[(2-{[2-(3-chlorophenyl)ethyl]amino}-2-oxoethyl)thio]propanoic acid (Example 32b), 1.97 g) according to the method of Example 26c) to yield the sub-titled compound (1.33 g).

¹H NMR δ(CDCL3) 7.25-7.17 (m, 3H), 7.12-7.07 (m, 1H), 3.75 (t, 2H), 2.92-2.76 (m, 6H), 2.70-2.61 (m, 4H), 1.86-1.79 (m, 2H)

### d) tert-Butyl [2-(3-chlorophenyl)ethyl]{2-[(3-hydroxypropyl)thio]ethyl}carbamate

The sub-titled compound was prepared from 3-[(2-{[2-(3-chlorophenyl)ethyl]amino}ethyl)thio]propan-1-ol (Example 32c), 1.33 g) according to the method of Example 26d) to yield the sub-titled compound (1.82 g).

m/e 274 ((M−BOC)+H+, 100%)

¹H NMR δ(CDCL3) 7.25-7.14 (m, 3H), 7.12-7.00 (m, 1H), 3.78-3.71 (m, 2H), 3.47-3.19 (m, 4H), 2.86-2.75 (m, 2H), 2.73-2.50 (m, 4H), 1.88-1.79 (m, 2H), 1.49-1.40 (m, 9H)

### e) tert-Butyl [2-(3-chlorophenyl)ethyl]{2-[(3-oxopropyl)thio]ethyl}carbamate

The sub-titled compound was prepared from tert-butyl [2-(3-chlorophenyl)ethyl]{2-[(3-hydroxypropyl)thio]ethyl}carbamate (Example 32d), 420 mg) according to the method of Example 9b). The crude product was purified flash chromatography on silica gel, eluting with 20% ethyl acetate in iso-hexane to yield the sub-titled compound (292 mg).

¹H NMR δ(CDCL3) 9.78 (d, 1H), 7.25-7.15 (m, 3H), 7.12-7.00 (m, 1H), 3.41 (d, 2H), 3.37-3.19 (m, 2H), 2.87-2.71 (m, 6H), 2.68-2.52 (m, 2H), 1.50-1.39 m, 9H)

### f) tert-Butyl [2-(3-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate

The titled compound was prepared from 7-[(1R)-2-Amino-1-hydroxyethyl]-4-hydroxy-1,3-benzothiazol-2(3H)-one hydrochloride (Example 4b), 150 mg) and tert-butyl [2-(3-chlorophenyl)ethyl]{2-[(3-oxopropyl)thio]ethyl}carbamate (Example 32e), 212 mg) according to the method of Example 5e). The crude product was purified by flash chromatography on silica gel, eluting with 1% concentrated aqueous ammonia and 10% methanol in dichloromethane to yield the titled compound (172 mg).

m/e 582 (M+H+, 100%)

### EXAMPLE 33

### 7-[(1R)-2-({3-[(2-{[2-(3-Chlorophenyl)ethyl]amino}ethyl)thio]propyl}amino)-1-hydroxyethyl]-4-hydroxy-1,3-benzothiazol-2(3H)-one

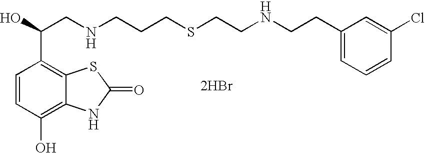

The titled compound was prepared from tert-butyl [2-(3-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate (Example 32f), 172 mg) according to the method of Example 27. The residue was dissolved in ethanol, acidified with 5% hydrobromic acid and concentrated in vacuo, not to dryness. It was triturated with acetonitrile and the solid collected by filtration to yield the titled compound (138 mg).

m/e 482 (M+H+, 100%)

¹H NMR δ(DMSO) 11.67 (s, 1H), 10.15 (s, 1H), 8.74-8.58 (m, 4H), 7.42-7.31 (m, 3H), 7.28-7.23 (m, 1H), 6.95 (d, 1H), 6.78 (d, 1H), 6.44 (s, 1H), 4.96-4.88 (m, 1H), 3.29-2.93 (m, 10H), 2.80 (t, 2H), 2.65 (t, 2H), 1.98-1.85 (m, 2H)

### EXAMPLE 34

### tert-Butyl [2-(2,3-dichlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate

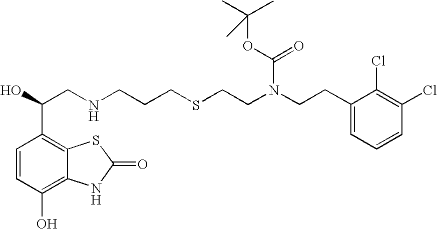

### a) Methyl 3-[(2-{[2-(2,3-dichlorophenyl)ethyl]amino}-2-oxoethyl)thio]propanoate

The sub-titled compound was prepared from [(3-methoxy-3-oxopropyl)thio]acetic acid (1.17 g) and [2-(2,3-dichlorophenyl)ethyl]amine (1.25 g) according to the method of Example 26a) to yield the sub-titled compound (2.29 g).

m/e 350 (M+H+, 100%)

### b) 3-[(2-{[2-(2,3-Dichlorophenyl)ethyl]amino}-2-oxoethyl)thio]propanoic acid

The sub-titled compound was prepared from methyl 3-[(2-{[2-(2,3-dichlorophenyl)ethyl]amino}-2-oxoethyl)thio]propanoate (Example 34a), 2.29 g) according to the method of Example 26b) to yield the sub-titled compound (2.20 g).

m/e 336 (M+H+, 100%)

¹H NMR δ(CDCL3) 7.36 (dd, 1H), 7.18-7.14 (m, 2H), 7.02-6.96 (m, 1H), 3.59 (q, 2H), 3.25 (s, 2H), 3.05 (t, 2H), 2.78-2.72 (m, 2H), 2.67-2.62 (m, 2H)

### c) 3-[(2-{[2-(2,3-Dichlorophenyl)ethyl]amino}ethyl)thio]propan-1-ol

The sub-titled compound was prepared from 3-[(2-{[2-(2,3-dichlorophenyl)ethyl]amino}-2-oxoethyl)thio]propanoic acid (Example 34b), 2.20 g) according to the method of Example 26c) to yield the sub-titled compound (1.16 g).

¹H NMR δ_{(CDCL3)} 7.34 (dd, 1H), 7.19-7.10 (m, 2H), 3.76 (t, 2H), 3.02-2.95 (m, 2H), 2.93-2.84 (m, 4H), 2.72-2.62 (m, 4H), 1.88-1.80 (m, 2H)

### d) tert-Butyl [2-(2,3-dichlorophenyl)ethyl]{2-[(3-hydroxypropyl)thio]ethyl}carbamate

The sub-titled compound was prepared from 3-[(2-{[2-(2,3-dichlorophenyl)ethyl]amino}ethyl)thio]propan-1-ol (Example 34c), 1.16 g) according to the method of Example 26d) to yield the sub-titled compound (1.53 g).

m/e 308 ((M−BOC)+H+, 100%)

¹H NMR δ(CDCL3) 7.38-7.31 (m, 1H), 7.18-7.04 (m, 2H), 3.78-3.71 (m, 2H), 3.46 (t, 2H), 3.41-3.21 (m, 2H), 3.07-2.95 (m, 2H), 2.73-2.51 (m, 4H), 1.89-1.81 (m, 2H), 1.49-1.35 (m, 9H)

### e) tert-Butyl [2-(2,3-dichlorophenyl)ethyl]{2-[(3-oxopropyl)thio]ethyl}carbamate

The sub-titled compound was prepared from tert-butyl [2-(2,3-dichlorophenyl)ethyl]{2-[(3-hydroxypropyl)thio]ethyl}carbamate (Example 34d), 460 mg) according to the method of Example 9b). The crude product was purified flash chromatography on silica gel, eluting with 20% ethyl acetate in iso-hexane to yield the sub-titled compound (340 mg).

¹H NMR δ(CDCL3) 9.78 (t, 1H), 7.38-7.31 (m, 1H), 7.19-7.03 (m, 2H), 3.47 (t, 2H), 3.39-3.21 (m, 2H), 3.08-2.94 (m, 2H), 2.88-2.53 (6H, m), 1.51-1.33 (9H, m)

### f) tert-Butyl [2-(2,3-dichlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate

The titled compound was prepared from 7-[(1R)-2-Amino-1-hydroxyethyl]-4-hydroxy-1,3-benzothiazol-2(3H)-one hydrochloride (Example 4b), 150 mg) and tert-butyl [2-(2,3-dichlorophenyl)ethyl]{2-[(3-oxopropyl)thio]ethyl}carbamate (Example 34e), 232 mg) according to the method of Example 5e). The crude product was purified by flash chromatography on silica gel, eluting with 1% concentrated aqueous ammonia and 10% methanol in dichloromethane to yield the titled compound (180 mg).

m/e 616 (M+H+, 100%)

### EXAMPLE 35

### 7-[(1R)-2-({2-[(3-{[2-(2,3-Dichlorophenyl)ethyl]amino}propyl)thio]ethyl}amino)-1-hydroxyethyl]-4-hydroxy-1,3-benzothiazol-2(3H)-one

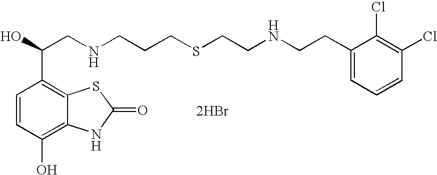

The titled compound was prepared from tert-butyl [2-(2,3-dichlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate (Example 34f), 180 mg) according to the method of Example C. The residue was dissolved in ethanol, acidified with 5% hydrobromic acid and concentrated in vacuo, not to dryness. It was triturated with acetonitrile and the solid collected by filtration to yield the titled compound (158 mg).

m/e 516 (M+H+, 100%)

¹H NMR δ(DMSO) 11.67 (s, 1H), 10.15 (s, 1H), 8.84-8.59 (m, 4H), 7.63-7.55 (m, 1H), 7.43-7.34 (m, 2H), 6.95 (d, 1H), 6.78 (d, 1H), 6.44 (s, 1H), 4.97-4.88 (m, 1H), 3.28-2.97 (m, 10H), 2.80 (t, 2H), 2.65 (t, 2H), 1.99-1.84 (m, 2H)

### EXAMPLE 36

### 7-((1R)-2-{[2-(3-{[2-(3-Chlorophenyl)ethyl]amino}prop oxy)ethyl]amino}-1-hydroxyethyl)-4-hydroxy-1,3-benzothiazol-2(3H)-one

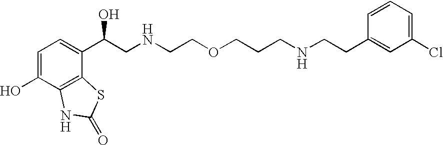

### a) Ethyl (3-hydroxypropoxy)acetate

Ethyl diazoacetate (5.5 g, 5.07 ml) was added dropwise over 1 h to a vigorously stirred solution of rhodium acetate dimer (110 mg) in 1,3-propanediol (55 g, 52.2 ml) and stirred for a further 20 h. The mixture was poured into water (1 L), extracted with ethyl acetate (×3), washed with water (×2), and brine, dried (Na₂SO₄), filtered and evaporated in vacuo to give the sub-titled compound as a light brown oil (3.9 g).

m/e 163 (M+H+, 100%)

¹H NMR (400 MHz, CDCl₃) δ 4.23 (q, 2H), 4.08 (s, 2H), 3.81 (q, 2H), 3.70 (t, 2H), 2.78 (t, 1H), 1.85 (quintet, 2H), 1.30 (t, 3H).

### b) Ethyl (3-{[(4-methylphenyl)sulfonyl]oxy}propoxy)acetate

Ethyl (3-hydroxypropoxy)acetate (Example 36a) (3.8 g) was dissolved in DCM (50 ml), triethylamine (2.85 g, 3.93 ml) added, followed by 4-methylbenzenesulfonyl chloride (4.9 g) and the reaction stirred for 20 h. Quenched with water, extracted with DCM, washed with water and brine, dried (Na₂SO₄), filtered and evaporated in vacuo. The residue was purified by flash column chromatography eluting with 25% EtOAc/isohexane to give the sub-titled compound as a colourless oil (5.7 g).

m/e 317 (M+H⁺, 100%)

¹H NMR (400 MHz, CDCl₃) δ 7.80 (d, 2H), 7.35 (d, 2H), 4.20 (q, 2H), 4.17 (t, 2H), 3.97 (s, 2H), 3.57 (t, 2H), 2.45 (s, 3H), 1.96 (m 2H), 1.28 (t, 3H).

c) Ethyl (3-{(tert-butoxycarbonyl)[2-(3-chlorophenyl)ethyl]amino}propoxy)acetate [2-(3-Chlorophenyl)ethyl]amine (650 mg, 581 μl) was dissolved in DMF (10 ml), triethylamine (850 mg, 1.171 ml) added, followed by ethyl (3-{[(4-methylphenyl)sulfonyl]oxy}propoxy)acetate (Example 36b) (880 mg) and the reaction stirred at 50° C. for 20 h. Cooled to 0° C., di-t-butyl dicarbonate (1.0 g) added and stirred for 1 h. Quenched with water, extracted with EtOAc, washed with water and brine, dried (Na₂SO₄), filtered and evaporated in vacuo. The residue was purified by flash column chromatography eluting with 20% EtOAc/isohexane to give the sub-titled compound as a colourless gum (700 mg).

m/e 400 (M+H⁺, 100%)

¹H NMR (400 MHz, CDCl₃) δ 7.19 (m, 3H), 7.07 (m, 1H), 4.21 (q, 2H), 4.05 (s, 2H), 3.40 (t, 2H), 3.53 (m, 2H), 3.25 (m, 2H), 2.80 (m, 2H), 1.83 (m, 2H), 1.43 (s, 9H), 1.28 (t, 3H).

### d) tert-Butyl [2-(3-chlorophenyl)ethyl][3-(2-hydroxyethoxy)propyl]carbamate

Ethyl (3-{(tert-butoxycarbonyl)[2-(3-chlorophenyl)ethyl]amino}propoxy)acetate (Example 36c) (650 mg) was dissolved in THF (5 ml), lithium borohydride (55 mg) added and stirred for 5 h. Quenched with water, extracted with ethyl acetate, washed with brine, dried (Na₂SO₄), filtered and evaporated in vacuo. The residue was purified by flash column chromatography eluting with 20% ethyl acetate/isohexane to give the sub-titled compound as a colourless oil (510 mg).

m/e 358 (M+H⁺, 100%)

¹H NMR (300 MHz, CDCl₃) δ 7.19 (m, 3H), 7.05 (m, 1H), 3.71 (m, 2H), 3.53 (m, 2H), 3.45 (t, 2H), 3.26 (m, 4H), 2.80 (m, 2H), 1.77 (m, 2H), 1.44 (s, 9H).

### e) 2-(3-{(tert-Butoxycarbonyl) [2-(3-chlorophenyl)ethyl]amino}propoxy)ethyl 4-methylbenzenesulfonate

tert-Butyl [2-(3-chlorophenyl)ethyl][3-(2-hydroxyethoxy)propyl]carbamate (Example d) (500 mg) was dissolved in DCM (10 ml), triethylamine (400 μl) added, followed by 4-methylbenzenesulfonyl chloride (300 mg) and the reaction stirred for 20 h. Quenched with water, extracted with ethyl acetate, washed with aqNaHCO₃ and brine, dried (Na₂SO₄), filtered and evaporated in vacuo. The residue was purified by flash column chromatography eluting with 20% ethyl acetate/isohexane to give the sub-titled compound as a colourless oil (500 mg).

m/e 513 (M+H⁺, 100%)

¹H NMR (300 MHz, CDCl₃) δ 7.79 (d, 2H), 7.33 (d, 2H), 7.19 (m, 3H), 7.06 (m, 1H), 4.15 (t, 2H), 3.59 (t, 2H), 3.39 (t, 2H), 3.36 (t, 2H), 3.16 (m, 2H), 2.78 (m, 2H), 2.43 (s, 3H), 1.72 (m, 2H), 1.42 (s, 9H).

### f) 7-[2-(2-{3-[2-(3-Chlorophenyl)-ethylamino]-propoxy}-ethylamino)-1-hydroxyethyl]-4-hydroxy-3H-benzothiazol-2-one

7-[(1R)-2-Amino-1-hydroxyethyl]-4-hydroxy-1,3-benzothiazol-2(3H)-one (100 mg) was dissolved in DMF (2 ml), triethylamine (75.1 mg, 103.4 μl) added, followed by 2-(3-{(tert-butoxycarbonyl)[2-(3-chlorophenyl)ethyl]amino}propoxy)ethyl 4-methylbenzenesulfonate (Example 36e) (190 mg) and the reaction stirred at 50° C. for 20 h. Quenched with water, extracted with ethyl acetate, washed with water and brine, dried (Na₂SO₄), filtered and evaporated in vacuo. The residue was dissolved in DCM (2 ml), TFA (2 ml) added and stirred for 1 h. Toluene (5 ml) added, evaporated in vacuo and the residue purified by reverse phase HPLC with aqTFA/CH₃CN as eluent. The solvent was evaporated in vacuo, the residue redissolved in 10% aqueous acetonitrile, aqHBr (500 μl) added and evaporated in vacuo to give the title compound as a white solid (80 mg).

m/e 466 (M+H⁺, 100%)

¹H NMR (400 MHz, CDCl₃) δ 7.18-7.04 (m, 3H), 6.98 (m, 1H), 6.79-6.70 (m, 1H), 6.60-6.50 (m, 1H), 4.71-4.60 (m, 1H), 3.53-3.24 (m, 4H), 2.90-2.55 (m, 8H), 1.65 (m, 2H), 1.23 (m, 2H).

### EXAMPLE 37

### tert-Butyl [2-(2,3-dichlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

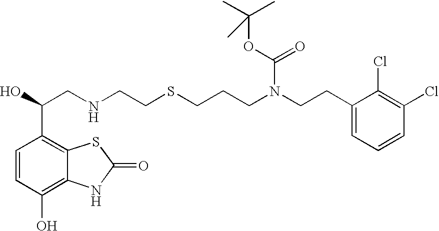

### a) tert-Butyl allyl[2-(2,3-dichlorophenyl)ethyl]carbamate

The sub-titled compound was prepared from tert-butyl [2-(2,3-dichlorophenyl)ethyl]carbamate (5.4 g) according to the method of Example 13a). The crude product was purified flash chromatography on silica gel, eluting with 5% ethyl acetate in iso-hexane to yield the sub-titled compound (3.9 g).

¹H NMR δ(DMSO-d6) 7.51 (d, 1H), 7.32-7.25 (m, 2H), 5.80-5.70 (m, 1H), 5.16-5.11 (m, 2H), 3.81-3.72 (m, 2H), 3.40 (s, 2H), 2.94 (t, 2H), 1.34-1.17 (m, 9H)

### b) tert-Butyl [2-(2,3-dichlorophenyl)ethyl]{3-[(2-hydroxyethyl)thio]propyl}carbamate

The sub-titled compound was prepared from tert-butyl allyl[2-(2,3-dichlorophenyl)ethyl]carbamate (Example 71a), 3.9 g) according to the method of Example 10a). The crude product was purified flash chromatography on silica gel, eluting with 50% ethyl acetate in iso-hexane to yield the sub-titled compound (2.1 g).

¹H NMR δ(DMSO-d6) 7.50 (d, 1H), 7.32-7.26 (m, 2H), 4.75 (t, 1H) 3.52-3.48 (m, 2H), 3.42 (s, 2H), 3.22 (s, 2H), 2.94 (t, 2H), 2.57-2.50 (m, 4H), 1.67 (t, 2H), 1.36-1.22 (m, 9H)

### c) tert-Butyl [2-(2,3-dichlorophenyl)ethyl]{3-[(2-oxoethyl)thio]propyl}carbamate

The sub-titled compound was prepared from tert-butyl [2-(2,3-dichlorophenyl)ethyl]{3-[(2-hydroxyethyl)thio]propyl}carbamate (Example 37b), 820 mg) according to the method of Example 17c). The crude product was purified flash chromatography on silica gel, eluting with 20% ethyl acetate in iso-hexane to yield the sub-titled compound (540 mg).

m/e 306 ((M−BOC)+H+, 100%)

### d) tert-Butyl [2-(2,3-dichlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

The titled compound was prepared from 7-[(1R)-2-Amino-1-hydroxyethyl]-4-hydroxy-1,3-benzothiazol-2(3H)-one hydrochloride (Example 4b), 350 mg) and tert-butyl [2-(2,3-dichlorophenyl)ethyl]{3-[(2-oxoethyl)thio]propyl}carbamate (Example 37c), 540 mg) according to the method of Example 5e). The crude product was purified by flash chromatography on silica gel, eluting with 1% concentrated aqueous ammonia and 10% methanol in dichloromethane then recrystallised from acetonitrile to yield the titled compound (190 mg).

m/e 616 (M+H+, 100%)

### EXAMPLE 38

### 7-[(1R)-2-({2-[(3-{[2-(2,3-Dichlorophenyl)ethyl]amino}propyl)thio]ethyl}amino)-1-hydroxyethyl]-4-hydroxy-1,3-benzothiazol-2(3H)-one dihydrochloride

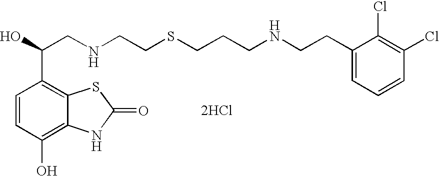

The titled compound was prepared from tert-butyl [2-(2,3-dichlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 37d), 190 mg) by dissolving it in dioxan (8 ml) and treating the solution with 4M HCl in dioxan (3 ml) with stirring at room temperature for 18 hours. The resultant precipitate was collected by filtration to yield the titled compound (160 mg).

m/e 516 (M+H+, 100%)

¹H NMR δ(DMSO-d6) 11.69 (s, 1H), 10.22 (s, 1H), 9.22 (s, 2H), 8.78 (s, 4H), 7.57 (d, 1H), 7.43-7.34 (m, 2H), 6.92 (d, 1H), 6.77 (d, 1H), 6.45 (s, 1H), 4.97 (d, 1H), 3.18 (s, 6H), 3.09-3.06 (m, 4H), 2.87-2.85 (m, 2H), 2.68 (t, 2H), 1.98-1.94 (m, 2H)

### EXAMPLE 39

### tert-Butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

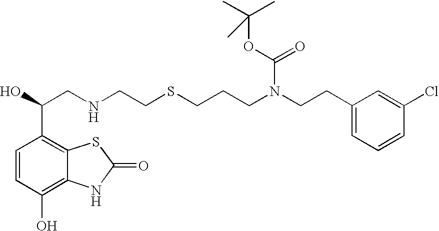

### a) tert-Butyl allyl[2-(3-chlorophenyl)ethyl]carbamate

The sub-titled compound was prepared from tert-butyl [2-(3-chlorophenyl)ethyl]carbamate (5.4 g) according to the method of Example 13a). The crude product was purified flash chromatography on silica gel, eluting with 5% ethyl acetate in iso-hexane to yield the sub-titled compound (3.9 g).

¹H NMR δ(DMSO-d6) 7.34-7.26 (m, 3H), 7.14 (d, 1H), 5.81-5.68 (m, 1H), 5.15-5.09 (m, 2H), 3.76 (s, 2H), 3.34 (t, 2H), 2.76 (t, 2H), 1.31 (m, 9H)

### b) tert-Butyl [2-(3-chlorophenyl)ethyl]{3-[(2-hydroxyethyl)thio]propyl}carbamate

The sub-titled compound was prepared from tert-butyl allyl[2-(3-chlorophenyl)ethyl]carbamate (Example 39a), 3.9 g) according to the method of Example 10a. The crude product was purified flash chromatography on silica gel, eluting with 50% ethyl acetate in iso-hexane to yield the sub-titled compound (3.8 g).

¹H NMR δ(DMSO-d6) 7.35-7.25 (m, 3H), 7.15 (d, 1H), 4.75 (t, 1H) 3.55-3.51 (m, 2H), 3.38 (t, 2H), 3.17 (t, 2H), 2.76 (t, 2H), 2.55-2.43 (m, 4H), 1.70 (t, 2H), 1.47-1.36 (m, 9H)

### c) tert-Butyl [2-(3-chlorophenyl)ethyl]{3-[(2-oxoethyl)thio]propyl}carbamate

The sub-titled compound was prepared from tert-butyl [2-(3-chlorophenyl)ethyl]{3-[(2-hydroxyethyl)thio]propyl}carbamate (Example 39b), 1200 mg) according to the method of Example 17c). The crude product was purified flash chromatography on silica gel, eluting with 20% ethyl acetate in iso-hexane to yield the sub-titled compound (760 mg).

m/e 272 ((M−BOC)+H+, 100%)

### d) tert-Butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

The titled compound was prepared from 7-[(1R)-2-Amino-1-hydroxyethyl]-4-hydroxy-1,3-benzothiazol-2(3H)-one hydrochloride (Example 4b, 540 mg) and tert-butyl [2-(3-chlorophenyl)ethyl]{3-[(2-oxoethyl)thio]propyl}carbamate (Example 39c), 760 mg) according to the method of Example 5e). The crude product was purified by flash chromatography on silica gel, eluting with 1% concentrated aqueous ammonia and 10% methanol in dichloromethane then recrystallised from acetonitrile to yield the titled compound (190 mg).

m/e 582 (M+H+, 100%)

### EXAMPLE 40

### 7-[(1R)-2-({2-[(3-{[2-(3-Chlorophenyl)ethyl]amino}propyl)thio]ethyl}amino)-1-hydroxyethyl]-4-hydroxy-1,3-benzothiazol-2(3H)-one dihydrochloride

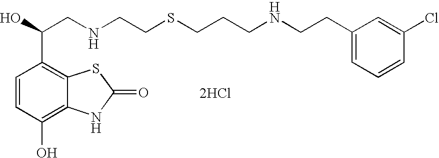

The titled compound was prepared from tert-butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 39d), 150 mg) by dissolving it in dioxan (6 ml) and treating the solution with 4M HCl in dioxan (2.5 ml) with stirring at room temperature for 18 hours. The resultant precipitate was collected by filtration to yield the titled compound (114 mg).

m/e 482 (M+H+, 100%)

¹H NMR δ(DMSO-d6) 11.70 (s, 1H), 10.24 (s, 1H), 9.21 (s, 3H), 8.82 (s, 1H), 7.42-7.31 (m, 3H), 7.25 (d, 1H), 6.93 (d, 1H), 6.81 (d, 1H), 6.45 (s, 1H), 4.99 (q, 1H), 3.15 (t, 4H), 3.04-2.87 (m, 6H), 2.84 (t, 2H), 2.68 (t, 2H), 1.94 (t, 2H)

### EXAMPLE 41

### tert-Butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)sulfonyl]propyl}carbamate

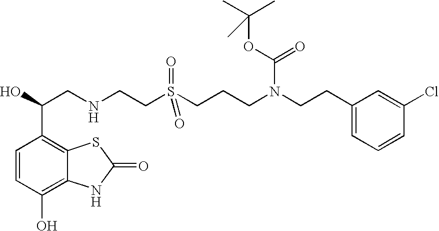

The titled compound was prepared from tert-butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 39d), (50 mg) by dissolving it in acetonitrile (15 ml) and a solution of oxone (80 mg) in water (15 ml) added for the mixture to be stirred overnight at room temperature. The mixture was concentrated and extracted with ethyl acetate and the organic layer shaken with brine then dried with sodium sulfate to be concentrated to a solid which was recrystallised from acetonitrile (21 mg).

m/e 614 (M+H+, 100%)

### EXAMPLE 42

### 7-[(1R)-2-({2-[(3-{[2-(3-Chlorophenyl)ethyl]amino}propyl)sulfonyl]ethyl}amino)-1-hydroxyethyl]-4-hydroxy-1,3-benzothiazol-2(3H)-one dihydrochloride

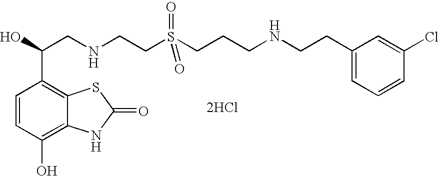

The titled compound was prepared from tert-butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)sulfonyl]propyl}carbamate (Example 41d), 21 mg) by dissolving it in dioxan (1 ml) and treating the solution with 4M HCl in dioxan (0.4 ml) with stirring at room temperature for 18 hours. The resultant precipitate was collected by filtration and purified by reverse phase HPLC using gradient elution of 5 to 60% acetonitrile in aqueous ammonia to yield the titled compound (1.3 mg).

m/e 514 (M+H+, 100%)

### EXAMPLE 43

### (+/−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

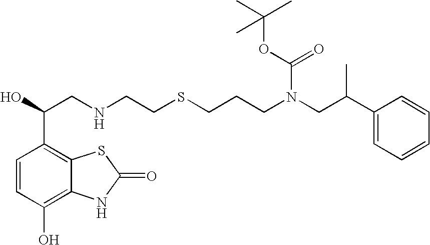

### a) (+/−)-tert-Butyl allyl[2-(phenyl)propyl]carbamate

The sub-titled compound was prepared from (+/−)-tert-butyl [2-(phenyl)propyl]carbamate (5.8 g) according to the method of Example 13a). The crude product was purified flash chromatography on silica gel, eluting with 5% ethyl acetate in iso-hexane to yield the sub-titled compound (3.7 g).

¹H NMR δ(DMSO-d6) 7.29 (t, 3H), 7.20 (d, 2H), 5.72-5.62 (m, 1H), 5.06 (d, 2H), 3.70 (s, 1H), 3.56 (s, 1H), 3.23 (s, 2H), 3.06-3.03 (m, 1H), 1.33 (m, 9H), 1.15, (d, 3H)

### b) (+/−)-tert-Butyl [2-(2-phenyl)propyl]{3-[(2-hydroxypropyl)thio]propyl}carbamate

The sub-titled compound was prepared from (+/−)-tert-butyl allyl[2-(phenyl)propyl]carbamate (Example 77a), 3.7 g) according to the method of Example 10a). The crude product was purified flash chromatography on silica gel, eluting with 50% ethyl acetate in iso-hexane to yield the sub-titled compound (3.3 g).

¹H NMR δ(DMSO-d6) 7.30-7.26 (d, 2H), 7.21-7.16 (m, 3H), 4.74 (t, 1H), 3.50 (q, 2H), 3.30 (s, 4H), 3.08 (t, 2H), 2.99-2.94 (m, 1H), 2.53 (t, 2H), 2.39 (t, 2H), 1.32 (s, 9H), 1.16 (s, 3H)

### c) (+/−)-tert-Butyl [2-(phenyl)propyl]{3-[(2-oxoethyl)thio]propyl}carbamate

The sub-titled compound was prepared from (+/−)-tert-butyl [2-(phenyl)propyl]{3-[(2-hydroxyethyl)thio]propyl}carbamate (Example 43b), 700 mg) according to the method of Example 17c). The crude product was purified flash chromatography on silica gel, eluting with 20% ethyl acetate in iso-hexane to yield the sub-titled compound (300 mg).

m/e 252 ((M−BOC)+H+, 100%)

### d) (+/−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

The titled compound was prepared from 7-[(1R)-2-Amino-1-hydroxyethyl]-4-hydroxy-1,3-benzothiazol-2(3H)-one hydrochloride (Example 4b), 220 mg) and (+/−)-tert-butyl [2-(phenyl)propyl]{3-[(2-oxoethyl)thio]propyl}carbamate (Example 43c), 300 mg) according to the method of Example 5e). The crude product was purified by flash chromatography on silica gel, eluting with 1% concentrated aqueous ammonia and 10% methanol in dichloromethane then recrystallised from acetonitrile to yield the titled compound (180 mg).

m/e 562 (M+H+, 100%)

### EXAMPLE 44

### (+/−)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one dihydrochloride

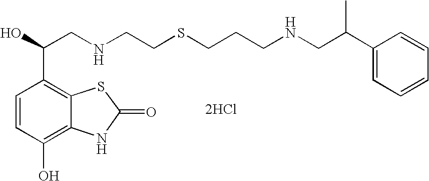

The titled compound was prepared from (+/−)-tert-butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 43d), 175 mg) by dissolving it in dioxan (6 ml) and treating the solution with 4M HCl in dioxan (2.5 ml) with stirring at room temperature for 18 hours. The resultant precipitate was collected by filtration to yield the titled compound (84 mg).

m/e 462 (M+H+, 100%)

¹H NMR δ(DMSO-d6) 11.69 (s, 1H), 10.21 (s, 1H), 9.18 (s, 1H), 8.92 (1H, s), 8.76 (s, 1H), 8.65 (1H, s), 7.37-7.31 (m, 3H), 7.29-7.25 (m, 2H), 6.91 (d, 1H), 6.78 (d, 1H), 6.45 (s, 1H), 4.96 (d, 1H), 3.39-3.22 (m, 1H), 3.10 (4H, t), 3.05 (s, 2H), 2.95 (quintet, 2H), 2.83 (q, 2H), 2.62 (t, 2H), 1.90 (quintet, 2H) 1.28 (d, 3H)

### EXAMPLE 45

### (R)-(+)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

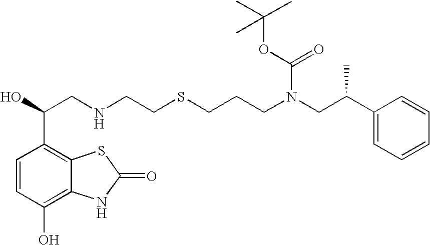

### a) (R)-(+)-tert-Butyl allyl[2-(phenyl)propyl]carbamate

The sub-titled compound was prepared from (R)-(+)-tert-butyl [2-(phenyl)propyl]carbamate (5.2 g) according to the method of Example 13a). The crude product was purified flash chromatography on silica gel, eluting with 5% ethyl acetate in iso-hexane to yield the sub-titled compound (4.2 g).

¹H NMR δ(DMSO-d6) 7.29 (t, 3H), 7.20 (d, 2H), 5.72-5.62 (m, 1H), 5.06 (d, 2H), 3.70 (s, 1H), 3.56 (s, 1H), 3.23 (s, 2H), 3.06-3.03 (m, 1H), 1.33 (9H, m), 1.15, (d, 3H)

### b) (R)-(+)-tert-Butyl [2-(2-phenyl)propyl]{3-[(2-hydroxypropyl)thio]propyl}carbamate

The sub-titled compound was prepared from (R)-(+)-tert-butyl allyl[2-(phenyl)propyl]carbamate (Example 45a), 4.2 g) according to the method of Example 10a). The crude product was purified flash chromatography on silica gel, eluting with 50% ethyl acetate in iso-hexane to yield the sub-titled compound (3.3 g).

¹H NMR δ(DMSO-d6) 7.30-7.26 (d, 2H), 7.21-7.16 (m, 3H), 4.74 (t, 1H), 3.50 (q, 2H), 3.30 (s, 4H), 3.08 (t, 2H), 2.99-2.94 (m, 1H), 2.53 (t, 2H), 2.39 (t, 2H), 1.32 (s, 9H), 1.16 (s, 3H)

### c) (R)-(+)-tert-Butyl [2-(phenyl)propyl]{3-[(2-oxoethyl)thio]propyl}carbamate

The sub-titled compound was prepared from (R)-(+)-tert-butyl [2-(phenyl)propyl]{3-[(2-hydroxyethyl)thio]propyl}carbamate (Example 45b), 1000 mg) according to the method of Example 17c). The crude product was purified flash chromatography on silica gel, eluting with 20% ethyl acetate in iso-hexane to yield the sub-titled compound (800 mg).

m/e 252 ((M−BOC)+H+, 100%)

### d) (R)-(+)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

The titled compound was prepared from 7-[(1R)-2-Amino-1-hydroxyethyl]-4-hydroxy-1,3-benzothiazol-2(3H)-one hydrochloride (Example 4b), 160 mg) and (R)-(+)-tert-butyl [2-(phenyl)propyl]{3-[(2-oxoethyl)thio]propyl}carbamate (Example 45c), 260 mg) according to the method of Example 5e). The crude product was purified by flash chromatography on silica gel, eluting with 1% concentrated aqueous ammonia and 10% methanol in dichloromethane then recrystallised from acetonitrile to yield the titled compound (65 mg).

m/e 562 (M+H+, 100%)

### EXAMPLE 46

### (R)-(+)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one dihydrochloride

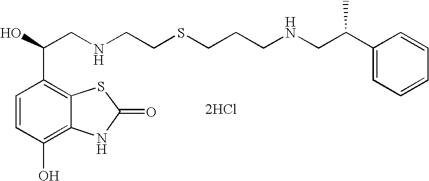

The titled compound was prepared from (R)-(+)-tert-butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 45d), 63 mg) by dissolving it in dioxan (2 ml) and treating the solution with 4M HCl in dioxan (0.8 ml) with stirring at room temperature for 18 hours. The resultant precipitate was collected by filtration to yield the titled compound (25 mg).

m/e 462 (M+H+, 100%)

¹H NMR δ(DMSO-d6) 11.69 (s, 1H), 10.21 (s, 1H), 9.18 (s, 1H), 8.92 (1H, s), 8.76 (s, 1H), 8.65 (s, 1H), 7.37-7.31 (m, 3H), 7.29-7.25 (m, 2H), 6.91 (d, 1H), 6.78 (d, 1H), 6.45 (s, 1H), 4.96 (d, 1H), 3.39-3.22 (m, 1H), 3.10 (4H, t), 3.05 (s, 2H), 2.95 (quintet, 2H), 2.83 (q, 2H), 2.62 (t, 2H), 1.90 (quintet, 2H) 1.28 (d, 3H)

### EXAMPLE 47

### (S)-(−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

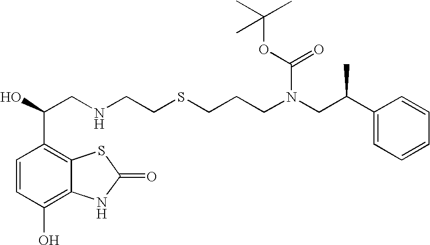

### a) (S)-(−)-tert-Butyl allyl[2-(phenyl)propyl]carbamate

The sub-titled compound was prepared from (S)-(−)-tert-butyl [2-(phenyl)propyl]carbamate (4.8 g) according to the method of Example 13a). The crude product was purified flash chromatography on silica gel, eluting with 5% ethyl acetate in iso-hexane to yield the sub-titled compound (4.2 g).

¹H NMR δ(DMSO-d6) 7.29 (t, 3H), 7.20 (d, 2H), 5.72-5.62 (m, 1H), 5.06 (d, 2H), 3.70 (s, 1H), 3.56 (s, 1H), 3.23 (s, 2H), 3.06-3.03 (m, 1H), 1.33 (m, 9H), 1.15, (d, 3H)

### b) (S)-(−)-tert-Butyl [2-(2-phenyl)propyl]{3-[(2-hydroxypropyl)thio]propyl}carbamate

The sub-titled compound was prepared from (S)-(−)-tert-butyl allyl[2-(phenyl)propyl]carbamate (Example 47a), 4.2 g) according to the method of Example 10a). The crude product was purified flash chromatography on silica gel, eluting with 50% ethyl acetate in iso-hexane to yield the sub-titled compound (3.4 g).

¹H NMR δ(DMSO-d6) 7.30-7.26 (d, 2H), 7.21-7.16 (m, 3H), 4.74 (t, 1H), 3.50 (q, 2H), 3.30 (s, 4H), 3.08 (t, 2H), 2.99-2.94 (m, 1H), 2.53 (t, 2H), 2.39 (t, 2H), 1.32 (s, 9H), 1.16 (s, 3H)

### c) (S)-(−)-tert-Butyl [2-(phenyl)propyl]{3-[(2-oxoethyl)thio]propyl}carbamate

The sub-titled compound was prepared from (S)-(−)-tert-butyl [2-(phenyl)propyl]{3-[(2-hydroxyethyl)thio]propyl}carbamate (Example 47b), 500 mg) according to the method of Example 17c). The crude product was purified flash chromatography on silica gel, eluting with 20% ethyl acetate in iso-hexane to yield the sub-titled compound (350 mg).

m/e 252 ((M−BOC)+H+, 100%)

### d) (S)-(−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

The titled compound was prepared from 7-[(1R)-2-Amino-1-hydroxyethyl]-4-hydroxy-1,3-benzothiazol-2(3H)-one hydrochloride (Example 4b), 150 mg) and (S)-(−)-tert-butyl [2-(phenyl)propyl]{3-[(2-oxoethyl)thio]propyl}carbamate (Example 47c), 250 mg) according to the method of Example 5e). The crude product was purified by flash chromatography on silica gel, eluting with 1% concentrated aqueous ammonia and 10% methanol in dichloromethane then recrystallised from acetonitrile to yield the titled compound (65 mg).

m/e 562 (M+H+, 100%)

### EXAMPLE 48

### (S)-(−)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one dihydrochloride

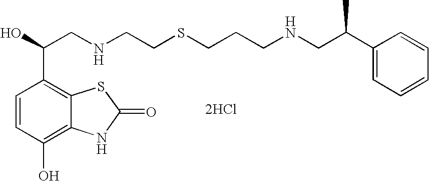

The titled compound was prepared from (S)-(−)-tert-butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 47d), 84 mg) by dissolving it in dioxan (2.5 ml) and treating the solution with 4M HCl in dioxan (1.0 ml) with stirring at room temperature for 18 hours. The resultant precipitate was collected by filtration to yield the titled compound (47 mg).

m/e 462 (M+H+, 100%)

¹H NMR δ(DMSO-d6) 11.69 (s, 1H), 10.21 (s, 1H), 9.18 (s, 1H), 8.92 (1H, s), 8.76 (s, 1H), 8.65 (s, 1H), 7.37-7.31 (m, 3H), 7.29-7.25 (m, 2H), 6.91 (d, 1H), 6.78 (d, 1H), 6.45 (s, 1H), 4.96 (d, 1H), 3.39-3.22 (m, 1H), 3.10 (4H, t), 3.05 (s, 2H), 2.95 (quintet, 2H), 2.83 (q, 2H), 2.62 (t, 2H), 1.90 (quintet, 2H) 1.28 (d, 3H)

### EXAMPLE 49

### tert-Butyl [2-methyl-2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

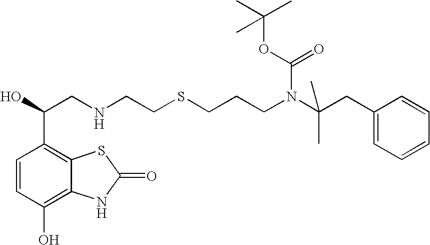

### a) tert-Butyl allyl[2-methyl-2-(phenyl)propyl]carbamate

The sub-titled compound was prepared by dissolving tert-butyl [2-methyl-2-(phenyl)propyl]carbamate (3.5 g) in DMF (15 ml). Sodium hydride 60% in oil (0.68 g) was rinsed with isohexane and then added to the solution and heated at 50 C for 2 hours to give a brown-red suspension. When cool, allyl bromide (1.9 ml) was added and the mixture was heated for a further 2 hours to complete the reaction. When cool, the mixture was quenched with brine and extracted with ethyl acetate (×3) with the combined extracts dried with sodium sulfate to concentrated to give a brown oil. This crude product was purified by flash chromatography on silica gel, eluting with 5% ethyl acetate in iso-hexane to yield the sub-titled compound (2.80 g).

¹H NMR δ(DMSO-d6) 7.27 (t, 2H), 7.21 (t, 1H), 7.10 (d, 2H), 5.54-5.47 (m, 1H), 4.94-4.87 (m, 2H), 3.45 (d, 2H), 3.07 (s, 2H), 1.45 (s, 9H), 1.36 (s, 6H)

### b) tert-Butyl [2-methyl-2-(2-phenyl)propyl]{3-[(2-hydroxypropyl)thio]propyl}carbamate

The sub-titled compound was prepared from tert-butyl allyl[2-methyl-2-(phenyl)propyl]carbamate (Example 49a), 2.80 g) according to the method of Example 10a). The crude product was purified flash chromatography on silica gel, eluting with 50% ethyl acetate in iso-hexane to yield the sub-titled compound (1.9 g).

¹H NMR δ_{(DMSO-d6)} 7.28 (t, 2H), 7.20 (t, 1H), 7.10 (d, 2H), 4.71 (t, 1H), 3.49-3.42 (m, 2H), 3.07 (t, 2H), 2.88 (t, 2H), 2.44 (t, 2H), 2.26 (t, 2H), 1.67 (s, 2H), 1.47 (s, 9H), 1.30 (s, 6H)

### c) tert-Butyl [2-methyl-2-(phenyl)propyl]{3-[(2-oxoethyl)thio]propyl}carbamate

The sub-titled compound was prepared from tert-butyl [2-methyl-2-(phenyl)propyl]{3-[(2-hydroxyethyl)thio]propyl}carbamate (Example 49b), 600 mg) according to the method of Example 17c). The crude product was purified flash chromatography on silica gel, eluting with 20% ethyl acetate in iso-hexane to yield the sub-titled compound (300 mg).

m/e 266 ((M−BOC)+H+, 100%)

### d) tert-Butyl [2-methyl-2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

The titled compound was prepared from 7-[(1R)-2-Amino-1-hydroxyethyl]-4-hydroxy-1,3-benzothiazol-2(3H)-one hydrochloride (Example 4b), 150 mg) and tert-butyl [2-methyl-2-(phenyl)propyl]{3-[(2-oxoethyl)thio]propyl}carbamate (Example 49c), 300 mg) according to the method of Example 5e). The crude product was purified by flash chromatography on silica gel, eluting with 1% concentrated aqueous ammonia and 10% methanol in dichloromethane then recrystallised from acetonitrile to yield the titled compound (60 mg).

m/e 576 (M+H+, 100%)

### EXAMPLE 50

### 7-[(1R)-2-({2-[(3-{[2-methyl-2-(phenyl)propyl]amino}propyl)thio]ethyl}amino)-1-hydroxyethyl]-4-hydroxy-1,3-benzothiazol-2(3H)-one dihydrochloride

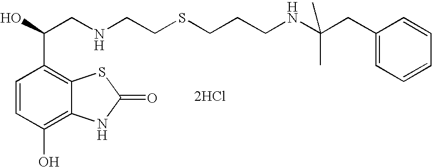

The titled compound was prepared from tert-butyl [2-methyl-2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (Example 49d), 60 mg) by dissolving it in dioxan (2 ml) and treating the solution with 4M HCl in dioxan (0.8 ml) with stirring at room temperature for 18 hours. The resultant precipitate was collected by filtration to yield the titled compound (15 mg)

m/e 476 (4+H+, 100%)

¹H NMR δ(DMSO-d6) 11.69 (s, 1H), 10.21 (s, 1H), 9.18 (s, 1H), 8.92 (1H, s), 8.76 (s, 1H), 8.65 (s, 1H), 7.26 (d, 1H), 7.20-7.15 (m, 4H), 6.86 (d, 1H), 6.68 (s, 1H), 4.56 (s, 1H), 3.39-3.22 (m, 6H), 2.54 (s, 6H) 1.62 (s, 2H), 0.95 (s, 6H)

### Biological Assay

### Experimental Procedures

### Cell Preparation

H292 cells were grown in RPMI (Roswell Park Memorial Institute) medium containing, 10% (v/v) FBS (foetal bovine serum) and 2 mM L-glutamine. Cells were grown in 225 cm2 flasks containing 25 mL media in a humidified incubator at 37° C., 5% CO₂. Cells were harvested from the flask and passaged at a 1 in 10 dilution once per week.

### Experimental Method

The media from flasks containing H292 cells was removed, rinsed with 10 mL PBS (phosphate buffered saline) and replaced with 10 mL Accutase™ cell detachment solution. Flasks were incubated for 15 minutes in a humidified incubator at 37° C., 5% CO₂. The cell suspension was counted and the cells re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed, washed twice with 100 μL assay buffer and replaced with 50 μL assay buffer. Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time test compounds (made up as ×4 concentrated stocks in assay buffer containing 4% (v/v) dimethylsulphoxide) were added and the cells were incubated for 10 minutes at room temperature. Final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using the AlphaScreen™ methodology. The frozen cell plate was thawed for 20 minutes on a plate shaker then 10 μL of the cell lysate was transferred to a 96-well white plate. 40 μL of mixed AlphaScreen™ detection beads (containing equal volumes of donor beads (pre-incubated with biotinylated cAMP in the dark for 30 minutes) and acceptor beads), was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations (made up in lysis buffer in a 96-well tissue-culture-treated plate and frozen/thawed alongside the test samples) and detected using the same protocol. Concentration response curves for agonists were constructed to determine both the pEC₅₀ and Intrinsic Activity. Intrinsic Activity was expressed as a fraction relative to the maximum activity determined for formoterol in each experiment. The results obtained for a representative selection of the compounds of the Examples are shown in Table 1 below.

TABLE 1

| Compound of | pEC₅₀ | Intrinsic Activity |
|---|---|---|
| Example 2 | 9.8 | 1.0 |
| Example 4 | 10.0 | 0.9 |
| Example 5 | 10.2 | 0.7 |
| Example 8 | 10.0 | 1.0 |
| Example 12 | 8.3 | 0.8 |

Alternative Adrenergic β2 Mediated cAMP Production

### Cell Preparation

H292 cells were grown in 225 cm2 flasks incubator at 37° C., 5% CO₂ in RPMI medium containing, 10% (v/v) FBS (foetal bovine serum) and 2 mM L-glutamine.

### Experimental Method

Adherent H292 cells were removed from tissue culture flasks by treatment with Accutase™ cell detachment solution for 15 minutes. Flasks were incubated for 15 minutes in a humidified incubator at 37° C., 5% CO₂. Detached cells were re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed and cells were washed twice with 100 μL assay buffer and replaced with 50 μL assay buffer (HBSS solution containing 10 mM HEPES pH7.4 and 5 mM glucose). Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time test compounds were added and the cells were incubated for 60 minutes at room temperature. The final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using AlphaScreen™ methodology. The frozen cell plate was thawed for 20 minutes on a plate shaker then 10 μL of the cell lysate was transferred to a 96-well white plate. 40 μL of mixed AlphaScreen™ detection beads pre-incubated with biotinylated cAMP, was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations. Concentration response curves for agonists were constructed and data was fitted to a four parameter logistic equation to determine both the pEC₅₀ and Intrinsic Activity. Intrinsic Activity was expressed as a fraction relative to the maximum activity determined for formoterol in each experiment. Results for compounds of the invention are to be found in Table 2.

### Selectivity Assays

### Adrenergic α1D

### Membrane Preparation

Membranes were prepared from human embryonic kidney 293 (HEK293) cells expressing recombinant human α1_{D} receptor. These were diluted in Assay Buffer (50 mM HEPES, 1 mM EDTA, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [³H]-prazosin (0.3 nM final concentration) and 10 μL of test compound (10× final concentration) were added to each test well. For each assay plate 8 replicates were obtained for [³H]-prazosin binding in the presence of 10 μL vehicle (10% (v/v) DMSO in Assay Buffer; defining maximum binding) or 10 μL BMY7378 (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-prazosin binding) were determined using serial dilutions typically in the range 0.1 nM to 10 μM. Data was fitted to a four parameter logistic equation to determine the compound potency, which was expressed as pIC50 (negative log molar concentration inducing 50% inhibition of [³H]-prazosin binding). Results are shown in Table 2 below.

### Adrenergic β1

### Membrane Preparation

Membranes containing recombinant human adrenergic beta 1 receptors were obtained from Euroscreen. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [¹²⁵I]-Iodocyanopindolol (0.036 μM final concentration) and 10 μL of test compound (10× final concentration) were added to each test well. For each assay plate 8 replicates were obtained for [¹²⁵I]-odocyanopindolol binding in the presence of 10 μL vehicle (10% (v/v) DMSO in Assay Buffer; defining maximum binding) or 10 μL Propranolol (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [¹²⁵I]-odocyanopindolol binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [¹²⁵I]-odocyanopindolol binding). Results are shown in Table 2 below.

### Dopamine D2

### Membrane Preparation

Membranes containing recombinant human Dopamine Subtype D2s receptors were obtained from Perkin Elmer. These were diluted in Assay Buffer (50 nm HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 30 μL [³H]-spiperone (0.16 nM final concentration) and 30 μL of test compound (10× final concentration) were added to each test well. For each assay plate 8 replicates were obtained for [³H]-spiperone binding in the presence of 30 μL vehicle (10% (v/v) DMSO in Assay Buffer; defining maximum binding) or 30 μL Haloperidol (10 μM final concentration; defining non-specific binding (NSB)). Membranes were then added to achieve a final volume of 300 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol. Total specific binding (B₀) was determined by subtracting the mean NSB from the mean maximum binding. NSB values were also subtracted from values from all other wells. These data were expressed as percent of B₀. Compound concentration-effect curves (inhibition of [³H]-spiperone binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [³H]-spiperone binding).

The results obtained for a representative selection of the compounds of the Examples are shown in Table 2 below.

### Onset Assay

Dunkin-Hartley guinea-pigs (between 200 g and 300 g on delivery) were supplied by a designated breeding establishment. The guinea-pigs were killed by cervical dislocation and the trachea removed. The adherent connective tissue was removed and each trachea cut into four rings. The tissue rings were then attached to an isometric transducer. The tissues were washed and a force of 1 g was applied to each ring. In all experiments a paired curve design was used. A priming dose of 1 μM methacholine was applied to the tissues. The tissues were then washed (three times, one minute between washes), the resting tension of 1 g was reapplied and the tissues were allowed to rest for 1 hour to equilibrate. Tissues were then contracted with 1 μM methacholine and once a steady response was obtained a cumulative concentration response curve to isoprenaline (10⁻⁹ M-10⁻⁵ M) was constructed. The tissues were then washed (three times, one minute between washes) and left to rest for an hour. At the end of the resting period the tissues were contracted with 1 μM methacholine and a p[A]₅₀ concentration of test compound added. Once the tissue had reached maximum relaxation, a 30×p[A]₅₀ concentration of test compound was added. Once the tissue response had reached a plateau, 10 μM sotalol was added to the bath to confirm that the relaxation was β₂ mediated

Data were collected using the AD Instruments chart 4 for windows software, which measured the maximum tension generated at each concentration of agonist.

For each concentration of the isoprenaline cumulative concentration curve, the response was calculated as % relaxation of the methacholine-induced contraction. A curve was plotted of log₁₀[agonist] (M) versus percentage inhibition of the methacholine-induced contraction. These data were then fitted to a non-linear regression curve fit. For each experiment, E/[A] curve data were fitted using a 4-parameter logistic function of the form:

$$E = β + \frac{( β - α ) \cdot {[ A ]}^{m}}{{[ A ]}^{m} + {[ A ]}_{50}^{m}}$$

E and [A] are the pharmacological effect (% relaxation) and concentration of the agonist respectively; α, β, [A]₅₀ and m are the asymptote, baseline, location and slope parameters, respectively. The p[A]₅₀ and IA of each isoprenaline curve was determined from this fit, to determine if the tissue was viable for generating an onset time for the test compounds.

For each p[A]₅₀ concentration of the test compound, the response was calculated as % relaxation of the methacholine-induced contraction. The results were plotted % relaxation against time and the time taken to reach a 90% relaxation value was calculated and recorded.

The addition of a 30×p[A]₅₀ concentration enabled determination of the maximum compound effect within the individual tissue. Hence, the % of the maximum compound effect at the p[A]₅₀ concentration was calculated and recorded.

### Pharmacokinetics in the Rat

A dose solution of the test compound was prepared using a suitable dose vehicle. The concentration of the compound in the dose solution was assayed by diluting an aliquot to a nominal concentration of 50 μg·ml⁻¹ and calibrating against duplicate injections of a standard solution and a QC standard at this concentration. Compounds were administered intravenously as a bolus into a caudal vein to groups of three 250-350 g rats (approximately 1 ml·kg⁻¹). For the oral dose, a separate group of 2 or 3 animals were dosed by oral gavage (3 ml·kg⁻¹). Delivered doses were estimated by weight loss. Food was not usually withdrawn from animals prior to dosing, although this effect was investigated if necessary.

Blood samples (0.25 ml) were taken into 1 ml syringes from the caudal vein, transferred to EDTA tubes and plasma was prepared by centrifugation (5 min at 13000 rpm) soon after sample collection, before storage at −20° C. Typical sampling times were 2, 4, 8, 15, 30, 60, 120, 180, 240, 300 (min) or until the terminal t1/2 was accurately described.

The concentration of the analyte(s) were determined in plasma by quantitative mass spectrometry. Standard and quality control stock solutions were prepared at a concentration 1 mg/ml in methanol. A range of standard and QC stocks produced by serial dilution were added to control rat plasma (50 μl). The range of concentrations covered the range of levels of analyte present in the rat samples. Standards, QCs and samples underwent liquid extraction using 50 μl of organic solvent and 100 μl of organic solvent containing an internal standard, chosen to closely resemble the analyte. The samples were then mixed by repeated inversion, stored at −20° C. for at least 1 h, and centrifuged at 3500 rpm in a centrifuge for 20 minutes. Aliquots (120 μl) of each sample were transferred for analysis using LC-MSMS. Standard and quality control samples covering the range of concentrations found in the test samples were within 25% of the nominal concentration.

Pharmacokinetic data analysis was achieved using WinNonlin. A standard non-compartmental analysis was used to estimate the parameters such as Tmax, Cmax, Lambda_z, t1/2_Lambda_z, AUCall, AUCINF(observed), Cl(observed), Vss(observed).

TABLE 2

| | | β2 Int | α1 bind | | |
|---|---|---|---|---|---|
| Ex No. | β2 pEC50 | Act | pIC50 | β1 bind pIC50 | D2 bind pIC50 |
| 1 | 10 | 1 | | 8.1 | 6.7 |
| 2 | 9.8 | 1 | 6 | 7.8 | |
| 3 | 9.9 | 0.9 | | 8.4 | 6.1 |
| 4 | 10 | 0.9 | 6.7 | 6.9 | 6.1 |
| 5 | 10.3 | 0.9 | | | |
| 6 | 10.1 | 0.9 | | 8.4 | 6.2 |
| 7 | 9.9 | 1.1 | 6.2 | 8.5 | 6 |
| 8 | 10.2 | 1 | | 8.1 | 6 |
| 9 | 10.1 | 1 | | 8.1 | 6 |
| 11 | 9.1 | 0.9 | 6.9 | 6.6 | 5.4 |
| 12 | 8.5 | 1 | | | |
| 14 | 8.4 | 0.9 | 7 | 7.3 | 5.6 |
| 16 | 7.9 | 0.7 | 5.8 | 6.9 | 5.4 |
| 18 | 8.5 | 0.8 | 6.6 | 7 | 6.3 |
| 20 | 9.2 | 0.8 | 7.6 | 6.9 | 5.8 |
| 24 | 9.4 | 0.8 | 6.8 | 7.1 | 6.2 |
| 27 | 9 | 1 | 6.9 | 7.2 | 6 |
| 29 | 9.5 | 0.9 | 7 | 7.6 | 5.4 |
| 31 | 9.4 | 0.8 | 7.9 | 7.4 | 5.8 |
| 33 | 8.9 | 1.1 | 7.4 | 6.9 | 6 |
| 35 | 8.8 | 1 | 6.9 | 7.1 | |
| 36 | 9.3 | 1 | 7.3 | 7 | 5.5 |
| 38 | 8.8 | 0.9 | 6.8 | 7.1 | 6.4 |
| 40 | 9 | 0.8 | 7.1 | 6.8 | 6.1 |
| 42 | 9.2 | 1 | 6.9 | 6.1 | 5 |
| 44 | 9.2 | 0.8 | 6.4 | 7.1 | 4.9 |
| 46 | 9.2 | 0.8 | 6.6 | 7.1 | 5.9 |
| 48 | 8.9 | 0.9 | 5.8 | 6.8 | 5.4 |
| 50 | 8.9 | 0.6 | 6.4 | 6.1 | 5.5 |

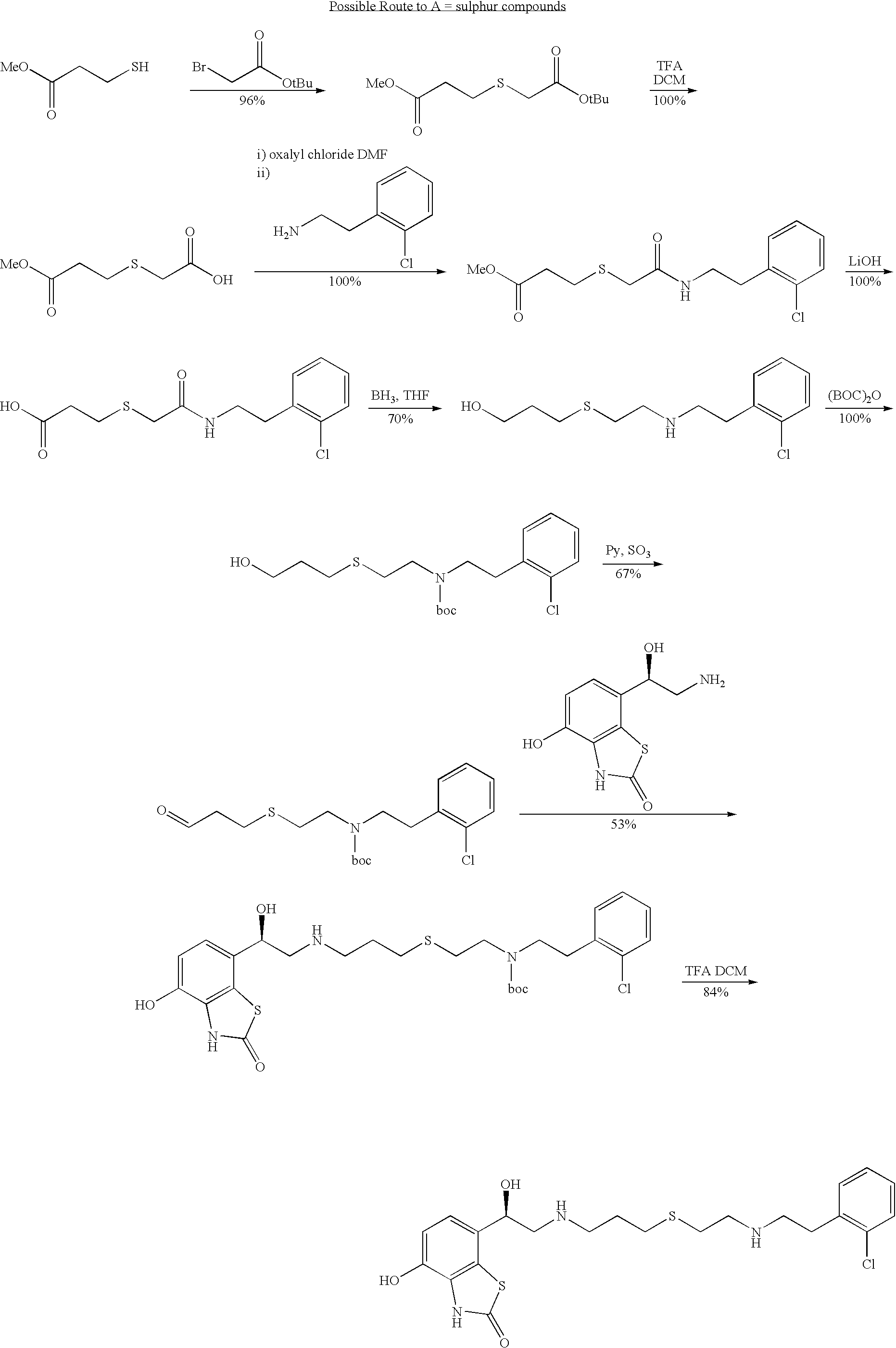

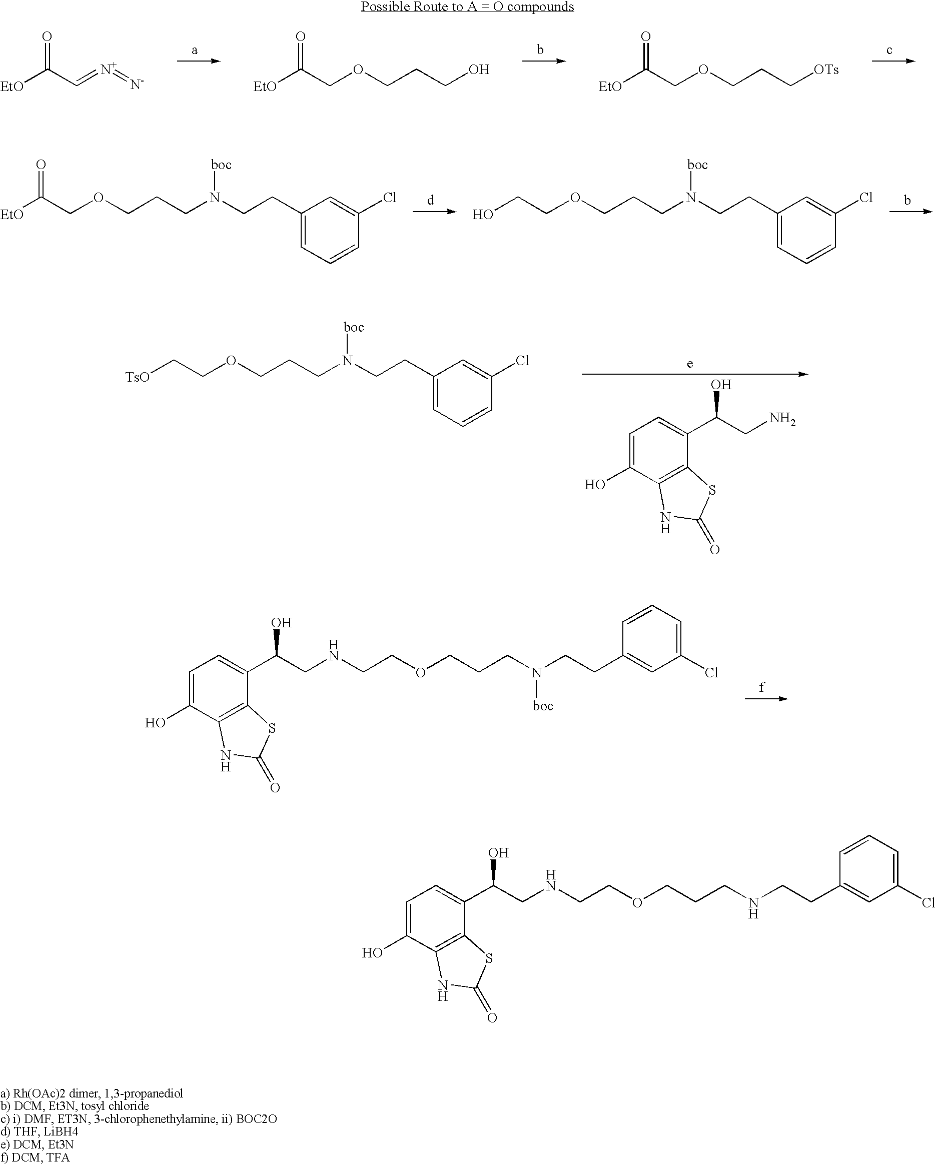

1. A compound of formula 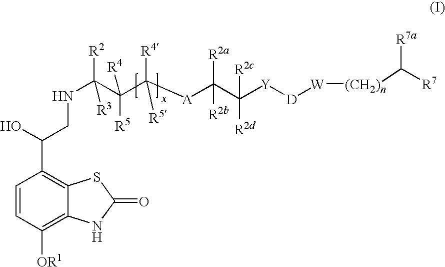
wherein
R¹ represents hydrogen;
each of R², R³, R⁴, R⁵, R^{4′} and R^{5′} independently represents hydrogen or C₁-C₆ alkyl;
x is 0 or 1;
A represents oxygen, sulphur, S(O) or S(O)₂;
D represents oxygen, sulphur or NR⁶;
W is a bond or CR^{6a}R^{6b};
n is an integer from 0 to 2;
R⁶ represents hydrogen, C₁-C₆ alkyl, C₁-C₆ alkoxycarbonyl or arylC₁-C₆alkyl;
Y is a bond, CR^{2e}R^{2f} or CR^{2g}R^{2h}CR^{2k}R^{2m};
R^{2a}, R^{2b}, R^{2c}, R^{2d}, R^{2e}, R^{2f}, R^{2g}, R^{2h}, R^{2k}, R^{2m}, R^{6a} and R^{6b} are, independently, hydrogen or C-₁-C₆ alkyl;
R^{7a} is hydrogen, C₁-C₆ alkyl or NHR^{7b};
R^{7b} is hydrogen, C₁-C₆ alkyl, C₁-C₆ alkoxycarbonyl or arylC₁-C₆alkyl;
R⁷ represents a 5- to 14-membered aromatic or heteroaromatic ring system optionally substituted by halogen, trifluoromethyl, hydroxyl, carboxyl, C₁-C₆ alkyl (optionally substituted by —NR¹⁰R¹¹), C₁-C₆ alkoxy (optionally substituted by —NR¹²R¹³), C₁-C₆ alkoxycarbonyl, —NR¹⁴R¹⁵, C₁-C₆ alkylcarbonylamino, C₁-C₆ alkylsulphonylamino, phenylsulphonylamino, —C(O)NHR¹⁶, —SO₂NHR¹⁷, C₀-C₆ alkyl-R¹⁸, or a phenyl or 5- to 6-membered heteroaromatic ring (each of which is optionally substituted by halogen, trifluoromethyl, hydroxyl, C₁-C₆ alkyl, C₁-C₆ alkoxy or —NR²¹R²²);
R¹⁰, R¹¹, R¹², R¹³, R¹⁴ and R¹⁵ each independently represent hydrogen or C₁-C₆ alkyl;
R¹⁶ represents hydrogen, C₁-C₆ alkyl, phenyl-C₀-C₆ alkyl or C₂-C₆ alkylene-NR¹⁹R²⁰;
either R¹⁹ and R²⁰ each independently represent hydrogen or C₁-C₆ alkyl, or R¹⁹ and R²⁰ together with the nitrogen atom to which they are attached form a 4- to 6-membered saturated heterocyclic ring optionally comprising a further ring heteroatom selected from nitrogen and oxygen;
R¹⁷ represents hydrogen, C₁-C₆ alkyl, phenyl-C₀-C₆ alkyl or C₂-C₆ alkylene-NR²³R²⁴;
either R²³ and R²⁴ each independently represent hydrogen or C₁-C₆ alkyl, or R²³ and R²⁴ together with the nitrogen atom to which they are attached form a 4- to 6-membered saturated heterocyclic ring optionally comprising a further ring heteroatom selected from nitrogen and oxygen;
R¹⁸ represents a saturated, 5- or 6-membered nitrogen-containing ring; and,
R²¹ and R²² each independently represent hydrogen or C₁-C₆ alkyl;
or a pharmaceutically acceptable salt thereof.

2. A compound as claimed in claim 1 wherein each of R², R³, R⁴, R⁵ and, if present, R^{4′} and R^{5′} represents hydrogen.

3. A compound as claimed in claim 1 wherein A represents oxygen, sulphur or S(O)₂.

4. A compound as claimed in claim 1 wherein D represents oxygen or NR⁶.

5. A compound as claimed in claim 1 wherein D represents NR⁶.

6. A compound as claimed in claim 1 wherein D is NR⁶ and A is sulphur.

7. A compound as claimed in claim 1 wherein R⁶ represents hydrogen, C₁-C₄ alkyl or C₁-C₄ alkoxycarbonyl.

8. A compound as claimed in claim 1 wherein x is 0.

9. A compound as claimed in claim 1 wherein Y is a bond or CR^{2e}R^{2f}, and n is 0.

10. A compound as claimed in claim 1 wherein R⁷ represents a 6- to 10-membered aromatic or heteroaromatic ring system optionally substituted by halogen, trifluoromethyl, hydroxyl, carboxyl, C₁-C₄ alkyl (optionally substituted by —NR¹⁰R¹¹), C₁-C₄ alkoxy (optionally substituted by —NR¹²R¹³), C₁-C₄ alkoxycarbonyl, —NR¹⁴R¹⁵, C₁-C₄ alkylcarbonylamino, C₁-C₄ alkylsulphonylamino, phenylsulphonylamino, —C(O)NHR¹⁶, —SO₂NHR¹⁷, C₀-C₄ alkyl-R¹⁸, phenyl or a 5- to 6-membered heteroaromatic ring.

11. A compound as claimed in claim 1 wherein n is 1 or 2, and W is CR^{6a}R^{6b}, wherein R^{6a} and R^{6b} are, independently, hydrogen or C₁₋₄ alkyl.

12. A compound as claimed in claim 1 wherein R^{7a} is NHR^{7b}, wherein R^{7b} is hydrogen, C₁-C₄ alkyl or C₁-C₆ alkoxycarbonyl.

13. A compound of formula (I) wherein: x is 0 or 1; A represents oxygen, sulphur or S(O)₂; D represents oxygen or NR⁶; Y is a bond or CH₂; W is CR^{6a}R^{6b}; n is 0; R¹, R², R³, R⁴, R⁵, R^{4′}, R^{5′}, R^{2a}, R^{2b}, R^{2c} and R^{2d} are all hydrogen; R⁶ represents hydrogen, C₁-C₄ alkyl or C₄ alkoxycarbonyl; R^{6a} and R^{6b} are, independently, hydrogen or C₁-C₄ alkyl; R⁷ represents a 6- to 10-membered aromatic ring system optionally substituted by halogen, C₁-C₄ alkyl, C₁-C₄ alkoxy or CF₃; and, R^{7a} is hydrogen, C₁-C₄ alkyl, NH(C₄ alkoxycarbonyl) or NH₂; or a pharmaceutically acceptable salt thereof.

14. A compound according to claim 1 that is:
4-Hydroxy-7-((1R)-1-hydroxy-2-{[3-({2-[2-(1-naphthyl)ethoxy]ethyl}thio)propyl]-amino}ethyl)-1,3-benzothiazol-2(3H)-one,
4-Hydroxy-7-((1R)-1-hydroxy-2-{[3-({2-[2-(1-naphthyl)ethoxy]ethyl}sulfonyl)propyl]-amino}ethyl)-1,3-benzothiazol-2(3H)-one,
4-Hydroxy-7-[(1R)-1-hydroxy-2-({3-[2-(2-phenylethoxy)ethoxy]propyl}amino)ethyl]-1,3-benzothiazol-2(3H)-one,
4-Hydroxy-7-((1R)-1-hydroxy-2-{[2-({2-[2-(1-naphthyl)ethoxy]ethyl}thio)ethyl]-amino}ethyl)-1,3-benzothiazol-2(3H)-one,
7-((1R)-2-{[3-({3-[2-(4-Bromophenyl)ethoxy]propyl}thio)propyl]amino}-1-hydroxyethyl)-4-hydroxy-1,3-benzothiazol-2(3H)-one,
4-Hydroxy-7-{(1R)-1-hydroxy-2-[(3-{[3-(2-phenylethoxy)propyl]thio}propyl)amino]ethyl}-1,3-benzothiazol-2(3H)-one,
4-Hydroxy-7-{(1R)-1-hydroxy-2-[(3-{2-[2-(1-naphthyl)ethoxy]ethoxy}propyl)amino]ethyl}-1,3-benzothiazol-2(3H)-one,
4-Hydroxy-7-{(1R)-1-hydroxy-2-[(2-{[3-(2-phenylethoxy)propyl]thio}ethyl)amino]ethyl}-1,3-benzothiazol-2(3H)-one,
4-Hydroxy-7-{(1R)-1-hydroxy-2-[(3-{[2-(2-phenylethoxy)ethyl]thio}propyl)amino]ethyl}-1,3-benzothiazol-2(3H)-one,
tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}(2-phenylethyl)carbamate,
4-Hydroxy-7-((1R)-1-hydroxy-2-{[2-({3-[(2-phenylethyl)amino]propyl}thio)ethyl]amino}ethyl)-1,3-benzothiazol-2(3H)-one,
4-Hydroxy-7-((1R)-1-hydroxy-2-{[2-({3-[methyl(2-phenylethyl)amino]propyl}-thio)ethyl]amino}ethyl)-1,3-benzothiazol-2(3H)-one,
tert-Butyl [2-(4-ethylphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(4-Ethylphenyl)ethyl]amino}propyl)thio]ethyl}amino)-1-hydroxyethyl]-4-hydroxy-1,3-benzothiazol-2(3H)-one,
tert-Butyl [2-(4-ethoxyphenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(4-Ethoxyphenyl)ethyl]amino}propyl)thio]ethyl}amino)-1-hydroxyethyl]-4-hydroxy-1,3-benzothiazol-2(3H)-one,
tert-Butyl {3-[(2-{[(2R)-2-hydroxy-2-(4-hydroxy-2-oxo-2,3-dihydro-1,3-benzothiazol-7-yl)ethyl]amino}ethyl)thio]propyl}{2-[3-(trifluoromethyl)phenyl]ethyl}carbamate,
4-Hydroxy-7-{(1R)-1-hydroxy-2-[(2-{[3-({2-[3-(trifluoromethyl)phenyl]ethyl}amino)propyl]thio}ethyl)amino]ethyl}-1,3-benzothiazol-2(3H)-one,
tert-Butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one,
tert-Butyl ((1S)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate,
7-((1R)-2-{[2-({3-[(2S)-2-Amino-2-phenylethoxy]propyl}thio)ethyl]amino}-1-hydroxyethyl)-4-hydroxy-1,3-benzothiazol-2(3H)-one,
tert-Butyl ((1R)-2-{3-[(2-{[(2R)-2-hydroxy-2-(4-hydroxy-2-oxo-2,3-dihydro-1,3-benzothiazol-7-yl)ethyl]amino}ethyl)thio]propoxy}-1-phenylethyl)carbamate,
7-((1R)-2-{[2-({3-[(2R)-2-Amino-2-phenylethoxy]propyl}thio)ethyl]amino}-1-hydroxyethyl)-4-hydroxy-1,3-benzothiazol-2(3H)-one,
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one,
tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2R)-2-phenylpropyl]carbamate,
4-Hydroxy-7-[(1R)-1-hydroxy-2-({3-[(2-{[(2R)-2-phenylpropyl]amino}ethyl)thio]propyl}amino)ethyl]-1,3-benzothiazol-2(3H)-one,
tert-Butyl {2-[(3-{[(2R)-2-hydroxy-2-(4-hydroxy-2-oxo-2,3-dihydro-1,3-benzothiazol-7-yl)ethyl]amino}propyl)thio]ethyl}[(2S)-2-phenylpropyl]carbamate,
4-Hydroxy-7-[(1R)-1-hydroxy-2-({3-[(2-{[(2S)-2-phenylpropyl]amino}ethyl)thio]propyl}amino)ethyl]-1,3-benzothiazol-2(3H)-one,
tert-Butyl [2-(2-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate,
7-[(1R)-2-({3-[(2-{[2-(2-Chlorophenyl)ethyl]amino}ethyl)thio]propyl}amino)-1-hydroxyethyl]-4-hydroxy-1,3-benzothiazol-2(3H)-one,
tert-Butyl [2-(3-chlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate,
7-[(1R)-2-({3-[(2-{[2-(3-Chlorophenyl)ethyl]amino}ethyl)thio]propyl}amino)-1-hydroxyethyl]-4-hydroxy-1,3-benzothiazol-2(3H)-one,
tert-Butyl [2-(2,3-dichlorophenyl)ethyl]{2-[(3-{[(2R)-2-hydroxy-2-(4-hydroxy-2-oxo-2,3-dihydro-1,3-benzothiazol-7-yl)ethyl]amino}propyl)thio]ethyl}carbamate,
7-[(1R)-2-({2-[(3-{[2-(2,3-Dichlorophenyl)ethyl]amino}propyl)thio]ethyl}amino)-1-hydroxyethyl]-4-hydroxy-1,3-benzothiazol-2(3H)-one,
7-((1R)-2-{[2-(3-{[2-(3-Chlorophenyl)ethyl]amino}propoxy)ethyl]amino}-1-hydroxyethyl)-4-hydroxy-1,3-benzothiazol-2(3H)-one,
tert-Butyl [2-(2,3-dichlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(2,3-Dichlorophenyl)ethyl]amino}propyl)thio]ethyl}amino)-1-hydroxyethyl]-4-hydroxy-1,3-benzothiazol-2(3H)-one,
tert-Butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
7-[(1R)-2-({2-[(3-{[2-(3-Chlorophenyl)ethyl]amino}propyl)thio]ethyl}amino)-1-hydroxyethyl]-4-hydroxy-1,3-benzothiazol-2(3H)-one,
tert-Butyl [2-(3-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)sulfonyl]propyl}carbamate,
7-[(1R)-2-({2-[(3-{[2-(3-Chlorophenyl)ethyl]amino}propyl)sulfonyl]ethyl}amino)-1-hydroxyethyl]-4-hydroxy-1,3-benzothiazol-2(3H)-one,
(+/−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
(+/−)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one,
(R)-(+)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
(R)-(+)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one,
(S)-(−)-tert-Butyl [2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate,
(S)-(−)-7-[(1R)-2-({2-[(3-{[2-(phenyl)propyl]amino}propyl)thio]ethyl}amino)-1-hydroxyethyl]-4-hydroxy-1,3-benzothiazol-2(3H)-one,
tert-Butyl [2-methyl-2-(phenyl)propyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate, or
7-[(1R)-2-({2-[(3-{[2-methyl-2-(phenyl)propyl]amino}propyl)thio]ethyl}amino)-1-hydroxyethyl]-4-hydroxy-1,3-benzothiazol-2(3H)-one,
or a pharmaceutically acceptable salt of any one thereof.

15. A process for the preparation of a compound of formula (I) or a pharmaceutically acceptable salt thereof as defined in claim 1 which comprises,
(a) reacting a compound of formula (II) 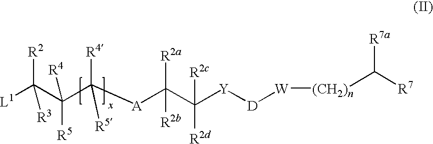
wherein L¹ represents a leaving group and the other variables are as defined in formula (I), with a compound of formula (III) or a suitable salt thereof 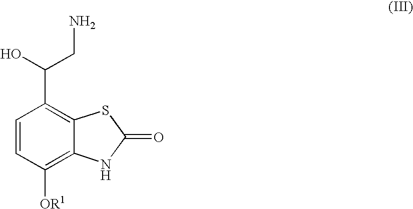
wherein R¹ is as defined in formula (I), in the presence of a base; or
(b) when R² and R³ each represent hydrogen, reacting a compound of formula (IV) 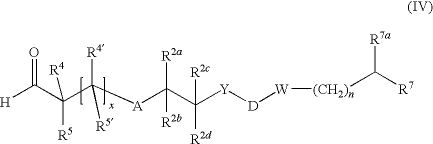
wherein the variables are as defined in formula (I), with a compound of formula (III) or a suitable salt thereof as defined in (a) above in the presence of a suitable reducing agent; or
(c) when R² and R³ each represent hydrogen, contacting a compound of formula (V) 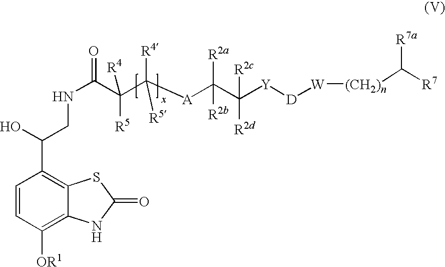
wherein the variables are as defined in formula (I) with a suitable reducing agent;
and optionally after (a), (b) or (c) carrying out one or more of the following:
converting the compound obtained to a further compound of the invention
forming a pharmaceutically acceptable salt of the compound.

16. A compound of formula 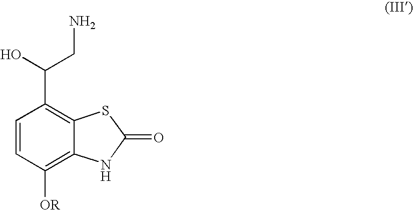
wherein R represents hydrogen or benzyl.

17. A compound of formula:

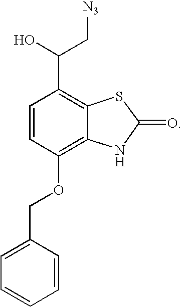

18. A pharmaceutical composition comprising a compound of formula (I) or a pharmaceutically acceptable salt thereof as claimed in claim 1 in association with a pharmaceutically acceptable adjuvant, diluent or carrier.

19. A process for the preparation of a pharmaceutical composition which comprises mixing a compound of formula (I) or a pharmaceutically acceptable salt thereof as claimed in claim 1 with a pharmaceutically acceptable adjuvant, diluent or carrier.

20-22. (canceled)

23. A method of treating, or reducing the risk of, a disease or condition in which modulation of β2 adrenoreceptor activity is beneficial which comprises administering to a patient in need thereof a therapeutically effective amount of a compound of formula (I) or a pharmaceutically acceptable salt thereof as claimed in claim 1.

24. A method of treating, or reducing the risk of, an inflammatory disease or condition which comprises administering to a patient in need thereof a therapeutically effective amount of a compound of formula (I) or a pharmaceutically acceptable salt thereof as claimed in claim 1.

25. A method according to claim 23, wherein the disease or condition is adult respiratory distress syndrome (ARDS), pulmonary emphysema, bronchitis, bronchiectasis, chronic obstructive pulmonary disease (COPD), asthma or rhinitis.

26. A combination comprising a compound of formula (I), or a pharmaceutically acceptable salt thereof, and one or more active agents selected from the list comprising:
a PDE4 inhibitor including an inhibitor of the isoform PDE4D;
a glucocorticoid receptor agonist;
a muscarinic receptor antagonist;
a modulator of chemokine receptor function; or,
an inhibitor of p38 kinase function.

27. A method according to claim 24, wherein the disease or condition is adult respiratory distress syndrome (ARDS), pulmonary emphysema, bronchitis, bronchiectasis, chronic obstructive pulmonary disease (COPD), asthma or rhinitis.
